(19) United States
(12) Patent Application Publication
(10) Pub. No.: US 2023/0218741 A1
(43) Pub. Date: Jul. 13, 2023

(54) SARS-COV-2 VACCINES FOR POPULATION-SCALE IMMUNITY

(71) Applicant: The Children's Hospital of Philadelphia, Philadelphia, PA (US)

(72) Inventors: John M. MARIS, Philadelphia, PA (US); Mark YARMARKOVICH, Philadelphia, PA (US); Alvin FARREL, Philadelphia, PA (US); John WARRINGTON, Philadelphia, PA (US)

(73) Assignee: The Children's Hospital of Philadelphia, Philadelphia, PA (US)

(21) Appl. No.: 17/915,986

(22) PCT Filed: Mar. 31, 2021
(86) PCT No.: PCT/US2021/025215

§ 371 Date: Sep. 29, 2022

Related U.S. Application Data
(60) Provisional application No. 63/002,963, filed on Mar. 31, 2020.

(51) Int. Cl. A61K 39/215; A61P 37/04

(52) U.S. Cl. CPC A61K 39/215; A61P 37/04; A61K 2039/53

(57) ABSTRACT

The present disclosure provides methods and compositions for SARS-CoV-2 vaccination strategy based on identification of both highly conserved regions of the virus and newly acquired adaptations that are presented by MHC class I and It across the vast majority of the population, are highly dissimilar from the human proteome, and are predicted B cell epitopes. This vaccination strategy specifically targets unique vulnerabilities of SARS-CoV-2 and should engage a robust adaptive immune response m the vast majority of the human population.

### REFERENCE TO RELATED APPLICATIONS

The present application claims the priority benefit of U.S. provisional application No. 63/002,963, filed Mar. 31, 2020, the entire contents of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Mar. 30, 2021, is named CHOPP0045WO_ST25.txt and is 118.8 kilobytes in size.

### BACKGROUND

### 1. Technical Field

The present disclosure relates generally to the fields of medicine, virology, and immunology. In certain aspects, the field of the disclosure concerns vaccine methods using viral T-cell epitopes.

### 2. Background

SARS-CoV-2 is the third coronavirus in the past two decades to acquire infectivity in humans and result in regional epidemics, with SARS-CoV-2 causing a global pandemic. The spike glycoprotein of SARS-CoV-2 dictates species tropism and is thought to bind to ACE2 receptors with 10-20-fold higher affinity than SARS-CoV in humans (Walls et al.; Wrapp et al., 2020). In addition, cleavage at a novel furin insertion site is predicted to facilitated membrane fusion and confer increased virulence, as has been previously reported with other viruses (Chen et al., 1998). Based on initial reports, infection of ACE2-expressing pneumocytes lining the pulmonary alveoli likely impairs release of surfactants that maintain surface tension, hindering the ability to prevent accumulation of fluid that may lead to acute respiratory distress syndrome (Xu et al., 2020; Zhang et al., 2020). The immune response of convalescent COVID-19 patients consists of antibody-secreting cells releasing IgG and IgM antibodies, increased follicular helper T cells, and activated CD4 and CD8 T cells (Thevaraj an et al., 2020), suggesting that a broad humoral and T cell driven immune response mediates the clearance of infection. The large size of the SARS-CoV-2 (˜29 kb) suggests that selection of optimal epitopes and reduction of unnecessary antigenic load for vaccination will be essential for safety and efficacy. The current SARS-CoV-2 pandemic has precipitated an urgent need to rapidly develop and deploy a safe and effective vaccine.

### SUMMARY

Here we describe an approach for prioritizing viral epitopes and present a list of peptides predicted to safely target the vulnerabilities of SARS-CoV-2, generating highly immunogenic epitopes on both MHC class I and II in the vast majority of the population, increasing the likelihood that prioritized epitopes will drive an adaptive memory response.

The vaccine concept provided herein focuses on: 1) stimulation of CD4 and CD8 T cells, 2) immunogenicity across the majority of human HLA alleles, 3) targeting both evolutionarily conserved regions, as well as newly divergent regions of the virus that increase infectivity, 4) targeting linear and conformational B cell epitopes, and 5) targeting viral regions with the highest degree of dissimilarity to the self-immunopeptidome, maximizing safety and immunogenicity. We present viral antigen minigenes for use in a multivalent vaccine construct that can be delivered by scalable techniques such as DNA, nucleoside mRNA, or synthetic peptides.

In one embodiment, provided herein are vaccine compositions comprising one or more antigens selected from SEQ ID NOS: 1-65 and 82 or a nucleic acid encoding one or more antigens selected from SEQ ID NOS: 1-65 and 82. In some aspects, the vaccine compositions comprise two or more antigens selected from SEQ ID NOS: 1-65 and 82.

In some aspects, the vaccine compositions comprise a fusion of two or more antigens selected from SEQ ID NOS: 1-65 and 82. In some aspects, the vaccine compositions comprise a linker between each antigen included in the vaccine. Each linker may be selected from GPGPG (SEQ ID NO: 79), AAY, HEYGAEALERAG (SEQ ID NO: 80), and EAAAK (SEQ ID NO: 81). In some aspects, the order of antigen epitopes and the linker used are chosen to prevent the formation of junctional epitopes having non-specific immunogenicity.

In some aspects, the vaccine composition comprises a signal peptide, such as, for example, an ER signal peptide (e.g., as encoded by nucleotide 724-789 of SEQ ID NO: 67), a lysosome signal peptide (e.g., as encoded by nucleotides 724-795 of SEQ ID NO: 68), and/or a secretion signal peptide (e.g., as encoded by nucleotides 724-780 of SEQ ID NO: 66).

In some aspects, the vaccine compositions comprise a nucleic acid sequence according to nucleotides 850-2322 of SEQ ID NO: 66, nucleotides 850-2445 of SEQ ID NO: 69, or nucleotides 850-2772 of SEQ ID NO: 72.

In some aspects, the vaccine compositions comprise a nucleic acid sequence according to nucleotides 724-2322 of SEQ ID NO: 66, nucleotides 724-2331 of SEQ ID NO: 67, nucleotides 724-2337 of SEQ ID NO: 68, nucleotides 724-2445 of SEQ ID NO: 69, nucleotides 724-2454 of SEQ ID NO: 70, nucleotides 724-2460 of SEQ ID NO: 71, nucleotides 724-2772 of SEQ ID NO: 72, nucleotides 724-2781 of SEQ ID NO: 73, or nucleotides 724-2787 of SEQ ID NO: 74. In some aspects, the nucleic acid sequence is an RNA sequence corresponding to the recited DNA sequence.

In some aspects, the vaccine compositions comprise a polypeptide encoded by nucleotides 850-2322 of SEQ ID NO: 66, nucleotides 850-2445 of SEQ ID NO: 69, or nucleotides 850-2772 of SEQ ID NO: 72. In some aspects, the vaccine compositions comprise a polypeptide encoded by nucleotides 724-2322 of SEQ ID NO: 66, nucleotides 724-2331 of SEQ ID NO: 67, nucleotides 724-2337 of SEQ ID NO: 68, nucleotides 724-2445 of SEQ ID NO: 69, nucleotides 724-2454 of SEQ ID NO: 70, nucleotides 724-2460 of SEQ ID NO: 71, nucleotides 724-2772 of SEQ ID NO: 72, nucleotides 724-2781 of SEQ ID NO: 73, or nucleotides 724-2787 of SEQ ID NO: 74.

In some aspects, the vaccine compositions further comprise an adjuvant, such as, for example, PADRE (e.g., as encoded by nucleotides 796-824 of SEQ ID NO: 66). In some aspects, the vaccine compositions further comprise a biological response modifier. In some aspects, the vaccine compositions further comprise a chemokine. In some aspects, the vaccine compositions further comprise a TLR agonist. The TLR agonist may drive activation of signals 1 and 2 in antigen presenting cells. The TLR agonist may be tetanus toxoid. In some aspects, said one or more antigens are comprised in an intact dendritic cell.

In some aspects, the vaccine compositions further comprise a second open reading frame encoding SARS-CoV-2 spike protein. In some aspects, the vaccine compositions further comprise a SARS-CoV-2 B cell antigen or nucleic acid encoding a SARS-CoV-2 B cell antigen. In some aspects, the vaccine composition is a DNA or mRNA having an open reading frame encoding the one or more antigen epitopes. In some aspects, the open reading frame is codon optimized.

In one embodiment, provided herein are methods of generating an anti-viral immune response is a subject, the methods comprising administering to the subject a vaccine composition according to any one of the present embodiments. In some aspects, the methods further comprise administering a second vaccine for SARS-CoV-2.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. Epitope Scoring along SARS-CoV-2 Proteome. (FIG. 1A) HLA presentation of 33mers across viral proteome. Representation of MHC Class I presentation (top) and MHC Class II presentation (bottom) reported as frequency of the population predicted to present each region of the viral proteome. (FIG. 1B) Scoring of each epitopes along the length of the proteome as compared to the epitopes derived from the normal human proteome presented across 84 HLA alleles, reported as normalized scores in which the highest scoring epitopes are maximally dissimilar to self-peptides derived from normal proteins (top). Scoring for genomic conservation against 9 cross-species coronaviruses and 1,024 human sequences, with highest scoring regions conserved across human and other mammalian coronaviruses (bottom). (FIG. 1C) Combined epitope score reported as sum of four above parameters (local maximum for epitopes with 90^{th} percentile total score). (FIG. 1D) Scoring of B cell epitopes for each amino acid for linear epitopes in for Spike, Envelope, and Matrix proteins (top) and conformational epitopes in Spike protein (bottom). (FIG. 1E) Combined scoring of 33mer epitopes as described in FIG. 1D. (FIG. 1F) Combined B and T cell epitope scoring in Spike, Envelope, and Matrix proteins. Receptor binding domain epitope highlighted with arrow and epitope containing furin cleavage site highlighted with arrow (FIG. 2).

FIG. 2. Proposed vaccine epitopes in SARS-CoV-2 Spike protein. Crystal structure of SARS-CoV-2 Spike protein trimer (PDB 6VYB) with two highlighted vaccine epitopes targeting novel acquired viral vulnerabilities. 1) SARS-CoV-2 receptor binding domain has up to 10-fold higher affinity binding to the ACE2 receptor as compared to previous coronaviruses. Using our analysis, we identify a high-ranking vaccine epitope within the receptor binding domain. 2) SARS-CoV-2 has acquired a novel furin cleavage site RRAR, along for increased infectivity due to improved membrane fusion (epitope containing the novel furin cleavage site highlighted in orange).

FIG. 3. Graphical abstract.

FIGS. 4A-B. Dissimilarity Scoring, related to STAR Methods Dissimilarity Scoring. (FIG. 4A) 3,524 viral epitopes (12,383 total peptide/WIC pairs) were compared against the normal human proteome. Non-anchor residues were used to calculate similarity scores based on amino acid classifications as described in methods. Residues in the same position of the viral and human peptides with a perfect match, similar amino acid classification, or different polarity, were assigned scores of five, two, or negative two, respectively. The viral peptide sequence is SEQ ID NO: 83; the human peptide sequence is SEQ ID NO: 84. (FIG. 4B) Each viral peptide/HLA pair was compared against the set of normal peptides presented on the same MHC. Dissimilarity score for each viral peptide was calculated by comparing against the most similar group of peptides with p<0.0001 and reported as the difference in Z-scores between the viral peptide and closest-scoring peptides. The viral peptide and perfect match peptide are SEQ ID NO: 83; the other peptide are represented by SEQ ID NOs: 85-98.

FIG. 5. Vector map including top-scoring 33mer peptides selected across all SARS-CoV-2 genes and ordered to minimize immunogenicity occurring at 33mer junctions as determined by population-scale HLA presentation algorithm of all potential peptides arising at junctions with all potential linker sequences to minimize immunogenicity. Constructs use signaling peptides to ER, lysosome, or secretion tags for presentation on WIC class I, MHC class II, and targeting by B cells, respectively. Construct employs a PADRE adjuvant sequence.

FIG. 6. ELISPOT IFN-γ results of vaccine construct composed of 33mers derived from spike protein (left) or across all SARS-CoV-2 genes (right). 15mer peptides overlapping by 5aa spanning the length of each construct were synthesized and split into four pools covering each ¼^{th} of the construct in order. Peptide pools were added to splenocytes collected from transgenic mice expressing human HLA-A*02:01 and spots counted for each mouse (represented by each dot). Splenocytes stimulated by peptides in pool A in spike vector shows significant IFN-γ production and by pools A, B, and D in the combination vector.

FIG. 7. IFN-γ is upregulated in CD8 T cells pulsed with pool A peptides in spike vaccine and in pools A, B, and D in combined vector, and not in controls.

FIG. 8. Vaccines induce potent CD8 T cell response as in FIG. 7, and CD4 responses observed in pool A in both spike and combined vaccine (vaccines were designed for presentation by human HLAs; vaccinated mice only express one human HLA recognized by CD8 and no alleles recognized by CD4). No IFN-γ release observed in scrambled vaccine composed 33mers selected at random from SARS-CoV-2.

FIG. 9. ELISPOT of expanded peptide mini-pools reveals overlapping sequences across 15mers. Expanded minipool of pool A reveals reactive peptides contained on multiple 15mers (gray sequences). Peptide 12 is SEQ ID NO: 99; peptide 14 is SEQ ID NO: 100; peptide 18 is SEQ ID NO: 101; peptide 22 is SEQ ID NO: 102; peptide 23 is SEQ ID NO: 103.

### DETAILED DESCRIPTION

Rapid deployment of antibody-based vaccination against SARS-CoV-2 raises a major concern in accelerating infectivity through Antibody-Dependent Enhancement (ADE), the facilitation of viral entry into host cells mediated by subneutralizing antibodies (those capable of binding viral particles, but not neutralizing them) (Dejnirattisai et al., 2016). ADE mechanisms have been described with other members of the Coronaviridae family (Wan et al., 2020; Wang et al., 2016), and it has already been suggested that some of the heterogeneity in COVID-19 cases may be due to ADE from prior infection from other viruses in the coronavirus family (Tetro, 2020). Although the T cell epitopes presented here are expected to be safe in vaccination, B cell epitopes should be further evaluated for their ability to induce neutralizing antibodies as compared to their potential to induce ADE. As it has been shown that T helper (Tx) cell responses are essential in humoral immune memory response (Alspach et al., 2019; McHeyzer-Williams, Okitsu, Wang, & McHeyzer-Williams, 2012), the T cell epitopes presented here are expected to activate CD4 T cells and drive memory B cell formation when paired with matched B cell epitopes.

The potential of a peptide-based vaccine to induce a memory B and T cell response is complicated by the diversity of HLA alleles across the human population. The HLA locus is the most polymorphic region of the human genome, resulting in differential presentation of antigens to the immune system in each individual. Therefore, individual epitopes may be presented in a mutually exclusive manner across individuals, confounding the ability to immunize with broadly presented antigens. While T cell receptors (TCRs) recognize linearized peptides anchored in the MHC groove, B cell receptors (BCRs) can recognize both linear and conformational epitopes, and are therefore difficult to predict without prior knowledge of a protein structure.

Optimally designed vaccines maximize immunogenicity towards regions of proteins that contribute most to protective immunity, while minimizing the antigenic load contributed by unnecessary protein domains that may result in autoimmunity, reactogenicity, or even enhanced infectivity.

Here we propose a vaccination strategy for SARS-CoV-2 based on identification of both highly conserved regions of the virus and newly acquired adaptations that are presented by MHC class I and II across the vast majority of the population, are highly dissimilar from the human proteome, and are predicted B cell epitopes. We present 65 peptide sequences that we expect to result in a safe and effective vaccine which can be rapidly tested in DNA, mRNA, or synthetic peptide constructs. These include epitopes that are contained within evolutionarily divergent regions of the spike protein reported to increase infectivity through increased binding to the ACE2 receptor, and within a novel furin cleavage site thought to increase membrane fusion. This vaccination strategy specifically targets unique vulnerabilities of SARS-CoV-2 and should engage a robust adaptive immune response in the vast majority of the human population.

### I. ASPECTS OF THE PRESENT INVENTION

Here we present a comprehensive immunogenicity map of the SARS-CoV-2 virus, highlighting 65 B and T cell epitopes (Table 1 and Table 2) from a diverse sampling of viral domains across all 9 SARS-CoV-2 genes. Based on our computational algorithm, we expect that the highest scoring peptides will result in safe and immunogenic T cell epitopes, and that B cell epitopes should be evaluated for safety and efficacy using methods previously reported (Wang et al., 2016). mRNA vaccines have been shown to be safe and effective in preclinical studies (Richner et al., 2017), with nucleoside RNAs shown to be effective without triggering RNA-induced immunogenicity (Pardi et al., 2017), while DNA vaccines have also been shown to be safe and protective (Dowd et al., 2016). Both DNA and mRNA vaccines are capable of being rapidly and efficiently manufactured at large scales. We suggest that a multivalent construct composed of the SARS-CoV-2 minigenes (presented in Tables 1-3) can be used in a DNA or mRNA vaccine for expression in antigen-presenting cells. These epitopes can be used in tandem with a TLR agonist such as tetanus toxoid (Zanetti, Ferreira, de Vasconcelos, & Han, 2019) to drive activation of signals 1 and 2 in antigen presenting cells. Constructs can be designed to contain a combination of optimal B and T cell epitopes, or deployed as a construct consisting of the top scoring T cell epitopes to be used in combination with the vaccines currently being developed targeting the Spike protein in order to drive the adaptive memory response. DNA vaccine sequences can also be codon optimized to increase CpG islands such as to increase TLR9 activation (Krieg, 2008).

The methods described here provide a rapid workflow for evaluating and prioritizing safe and immunogenic regions of a viral genome for use in vaccination. With the third epidemic in the past two decades underway, and all originating from a coronavirus family virus, these viruses will continue to threaten the human population, and necessitate the need for prophylactic measures against future outbreaks. A subset of the epitopes selected here are derived from viral regions sharing a high degree of homology with other viruses in the family, and thus we expect these evolutionarily conserved regions to be essential in the infectivity and replicative lifecycle across the coronavirus family, suggesting that an immune response against the epitopes listed herein may provide more broadly protective immunity against other coronaviruses. Additionally, we describe epitopes containing the newly acquired features of SARS-CoV-2 that confer evolutionary advantages in viral spread and infectivity. In addition, an immunogenicity map can be used to customize epitopes based on the HLA frequencies of specific populations. Though here we suggest the use of 33mers based on optimal MHC presentation across the population, these methods can be applied to evaluate k-mers of various sizes depending on desired application.

Antigenic burden from epitopes that do not contribute to viral protection can cause autoimmune reactions, reactogenicity, detract from the efficacy of the virus, or result in ADE. To mitigate these effects a priori, we selected maximally immunogenic epitopes with the highest degree of dissimilarity to the self-proteome such as to minimize the potential of cross-reactivity that can lead to adverse reaction or minimize the efficacy of the vaccine. In addition to the predicted safety of these epitopes stemming from lack of potentially cross-reactive normal proteins, we expect that a greater repertoire of viral antigen-specific T cells will exist due to lack of negative thymic selection. We prioritize epitopes with maximal dissimilarity from the human proteome, however, many other SARS-CoV-2 peptides show identical or nearly identical peptides presented on MHC derived from normal proteins, suggesting their use in vaccination could result in an autoimmune response. The 65 epitopes presented here can be expressed in a ˜6.3 kb construct and coupled with the safe and rapid production of synthetic DNA, mRNA, and peptide vaccines. As SARS-CoV-2 has precipitated the need to develop novel approaches to rapidly deploy vaccines in pandemic situations (Lurie, Saville, Hatchett, & Halton, 2020), we suggest that this comprehensive analysis can be incorporated into a process that can be rapidly deployed in when future novel viral pathogens emerge.

TABLE 1
List of highest scoring viral epitopes suggested for vaccination based on MHC
class I population-scale presentation, MHC class II population presentation,
similarity score, and homology score across 9 mammal species and 1,024 human
SARS-CoV-2 cases. Last column represents total score across all parameters,
highlighting epitope S_462 in S protein containing novel receptor binding
sites (Shang et al., 2020).

| | | HLA | | | HLA | | | | | | | B |
|---|---|---|---|---|---|---|---|---|---|---|---|---|
| | | Class | HLA | | Class | HLA | | | | | | and |
| | | I | Class | | II | Class | HLA | | Con- | | | T |
| | | Popu- | I | | Popu- | II | Class | Dis- | ser- | | B | cell |
| Gene | | lation | Al- | HLA | lation | Al- | II | simi- | va- | Com- | cell | To- |
| Posi- | | Presen- | leles | Class I | Presen- | leles | bind- | larity | tion | bined | total | tal |
| tion | Epitope | tation | Bound | binders | tation | Bound | ers | Score | Score | Score | score | % |
| ORF1ab | IAMSAF | 98.6% | 74 | HLA-A: | 82.1% | 24 | HLA- | 0.82 | 0.96 | 3.59 | NA | NA |
| _3619 | AMMFV | | | 0101, 0201, | | | DRB1: | | | | | |
| | KHKHAF | | | 0202, 0202, | | | 0101, | | | | | |
| | LCLFLLP | | | 0203, 0205, | | | 0401, | | | | | |
| | SLATVAY | | | 0205, 0206, | | | 0402, | | | | | |
| | FN | | | 0207, 0211, | | | 0403, | | | | | |
| | (SEQ | | | 0211, 0212, | | | 0404, | | | | | |
| | ID | | | 0216, 0217, | | | 0405, | | | | | |
| | NO: | | | 0217, 0219, | | | 0801, | | | | | |
| | 1) | | | 0301, 1101, | | | 0901, | | | | | |
| | | | | 2301, 2403, | | | 1001, | | | | | |
| | | | | 2403, 2501, | | | 1101, | | | | | |
| | | | | 2601, 2602, | | | 1301, | | | | | |
| | | | | 2603, 2902, | | | 1602 | | | | | |
| | | | | 3001, 3002, | | | HLA- | | | | | |
| | | | | 3201, 3201, | | | DPA10- | | | | | |
| | | | | 3207, 6601, | | | DPB10: | | | | | |
| | | | | 6601, 6801, | | | 103- | | | | | |
| | | | | 6801, 6802, | | | 201, | | | | | |
| | | | | 6823, 6823, | | | 103- | | | | | |
| | | | | 6901, 8001 | | | 401, | | | | | |
| | | | | HLA-B: | | | 103- | | | | | |
| | | | | 0801, 0802, | | | 402, | | | | | |
| | | | | 0803, 1501, | | | 103- | | | | | |
| | | | | 1502, 1503, | | | 601, | | | | | |
| | | | | 1503, 1509, | | | 201- | | | | | |
| | | | | 1517, 3501, | | | 101, | | | | | |
| | | | | 3501, 3503, | | | 201- | | | | | |
| | | | | 3801, 3801, | | | 501, | | | | | |
| | | | | 4013, 4506, | | | 301- | | | | | |
| | | | | 4601, 4801, | | | 402 | | | | | |
| | | | | 5101, 5301, | | | HLA- | | | | | |
| | | | | 5801, 5802, | | | DQA10- | | | | | |
| | | | | 7301, 8301 | | | DQB10: | | | | | |
| | | | | HLA-C: | | | 101- | | | | | |
| | | | | 0303, 0401, | | | 501, | | | | | |
| | | | | 0602, 0701, | | | 102- | | | | | |
| | | | | 0702, 0802, | | | 602, | | | | | |
| | | | | 1203, 1402, | | | 103- | | | | | |
| | | | | 1502 | | | 603, | | | | | |
| | | | | | | | 501- | | | | | |
| | | | | | | | 201, | | | | | |
| | | | | | | | 501- | | | | | |
| | | | | | | | 301 | | | | | |
| S_129 | KVCEFQ | 0.98455541 | 58 | HLA-A: | 39.0% | 9 | HLA- | 0.60 | 0.83 | 2.80 | 1.14 | 91% |
| | FCNDPF | | | 0101, 0201, | | | DRB1: | | | | | |
| | LGVYYH | | | 0202, 0206, | | | 0403, | | | | | |
| | KNNKS | | | 0211, 0212, | | | 1302, | | | | | |
| | WMESE | | | 0216, 0217, | | | 0405, | | | | | |
| | FRVYS | | | 0301, 0302, | | | 0404 | | | | | |
| | (SEQ | | | 1101, 2301, | | | HLA- | | | | | |
| | ID | | | 2402, 2403, | | | DPA10- | | | | | |
| | NO: | | | 2602, 3001, | | | DPB10: | | | | | |
| | 48) | | | 3101, 3207, | | | 103- | | | | | |
| | | | | 6601, 6823, | | | 201, | | | | | |
| | | | | 6823, 6901, | | | 103- | | | | | |
| | | | | 8001 | | | 401, | | | | | |
| | | | | HLA-B: | | | 103- | | | | | |
| | | | | 0803, 1501, | | | 601, | | | | | |
| | | | | 1502, 1503, | | | 301- | | | | | |
| | | | | 1509, 1509, | | | 402 | | | | | |
| | | | | 1517, 1801, | | | HLA- | | | | | |
| | | | | 2720, 3501, | | | DQA10- | | | | | |
| | | | | 3701, 3801, | | | DQB10: | | | | | |
| | | | | 3901, 4001, | | | 102- | | | | | |
| | | | | 4002, 4013, | | | 602 | | | | | |
| | | | | 4403, 4501, | | | | | | | | |
| | | | | 4506, 4601, | | | | | | | | |
| | | | | 4801, 5801, | | | | | | | | |
| | | | | 7301, 7301 | | | | | | | | |
| | | | | HLA-C: | | | | | | | | |
| | | | | 0303, 0401, | | | | | | | | |
| | | | | 0501, 0602, | | | | | | | | |
| | | | | 0701, 0702, | | | | | | | | |
| | | | | 0802, 1203, | | | | | | | | |
| | | | | 1402, 1502, | | | | | | | | |
| S_252 | GDSSSG | 0.95663996 | 53 | HLA-A: | 68.9% | 15 | HLA- | 0.48 | 0.71 | 2.84 | 0.76 | 81% |
| | WTAGA | | | 0101, 0201, | | | DRB1: | | | | | |
| | AAYYVG | | | 0202, 0203, | | | 0101, | | | | | |
| | YLQPRTF | | | 0205, 0206, | | | 0401, | | | | | |
| | LLKYNE | | | 0211, 0212, | | | 0402, | | | | | |
| | NGT | | | 0216, 0217, | | | 0404, | | | | | |
| | (SEQ | | | 0219, 2403, | | | 0405, | | | | | |
| | ID | | | 2501, 2601, | | | 0701, | | | | | |
| | NO: | | | 2602, 2603, | | | 0901, | | | | | |
| | 41) | | | 2603, 2902, | | | 1001, | | | | | |
| | | | | 3002, 3207, | | | 1301, | | | | | |
| | | | | 3301, 6601, | | | 1501, | | | | | |
| | | | | 6801, 6802, | | | 1602 | | | | | |
| | | | | 6823, 6823, | | | HLA- | | | | | |
| | | | | 6901, 8001, | | | DPA10- | | | | | |
| | | | | 8001 | | | DPB10: | | | | | |
| | | | | HLA-B: | | | 103- | | | | | |
| | | | | 0801, 0802, | | | 301, | | | | | |
| | | | | 0803, 1402, | | | 301- | | | | | |
| | | | | 1502, 1503, | | | 402 | | | | | |
| | | | | 1517, 3501, | | | HLA- | | | | | |
| | | | | 4013, 4501, | | | DQA10- | | | | | |
| | | | | 4506, 5703, | | | DQB10: | | | | | |
| | | | | 5801, 8301 | | | 102- | | | | | |
| | | | | HLA-C: | | | 602, | | | | | |
| | | | | 0303, 0401, | | | 501- | | | | | |
| | | | | 0602, 0701, | | | 301 | | | | | |
| | | | | 0702, 0802, | | | | | | | | |
| | | | | 1203, 1402, | | | | | | | | |
| | | | | 1502 | | | | | | | | |
| S_462 | KPFERDI | 74.8% | 27 | A: | 18.7% | 5 | DRB1: | 0.51 | 0.77 | 2.21 | 1.29 | 75.2% |
| | STEIYQA | | | 0206, 2402, | | | 0701, | | | | | |
| | GSTPCN | | | 2403, 2403, | | | 0801, | | | | | |
| | GVEGFN | | | 3207, 6601, | | | 1101, | | | | | |
| | CYFPLQS | | | 6802, 6823 | | | 1602 | | | | | |
| | (SEQ | | | B: | | | | | | | | |
| | ID | | | 0802, 1402, | | | | | | | | |
| | NO: | | | 1502, 1503, | | | | | | | | |
| | 64) | | | 2720, 3503, | | | | | | | | |
| | | | | 4002, 4013, | | | DPA10- | | | | | |
| | | | | 4201, 4506, | | | DPB10: | | | | | |
| | | | | 4801, 8301 | | | 201- | | | | | |
| | | | | C: | | | 501 | | | | | |
| | | | | 0401, 0401, | | | | | | | | |
| | | | | 0702, 1203, | | | | | | | | |
| | | | | 1402, 1402 | | | | | | | | |

TABLE 2

| | | HLA | | | HLA | | | | | |
|---|---|---|---|---|---|---|---|---|---|---|
| | | Class | HLA | | Class | HLA | | | | |
| | | I | Class | | II | Class | | | Con- | |
| | | Popu- | I | HLA | Popu- | II | HLA | | ser- | |
| Gene | | lation | Al- | Class | lation | Al- | Class | Dissimi- | va- | Com- |
| Posi- | | Presen- | leles | I | Presen- | leles | II | larity | tion- | bined |
| tion | Epitope | tation | Bound | binders | tation | Bound | binders | Score | Score | Score |
| ORF1ab | IAMSAF | 98.6% | 74 | A6901, B3501, | 82.1% | 24 | DRB1_0101, | 0.81769763 | 0.96 | 3.59 |
| _3619 | AMMF | | | B4601, C0303, | | | DRB1_0401, | 4 | | |
| | VKHKH | | | C1502, A2403, | | | DRB1_0402, | | | |
| | AFLCLFL | | | A2902, A3201, | | | DRB1_0403, | | | |
| | LPSLAT | | | A8001, B0802, | | | DRB1_0404, | | | |
| | VAYFN | | | B0803, B1501, | | | DRB1_0405, | | | |
| | (SEQ ID | | | B1502, B1503, | | | DRB1_0801, | | | |
| | NO: 1) | | | B4506, A0202, | | | DRB1_0901, | | | |
| | | | | A0206, A3001, | | | DRB1_1001, | | | |
| | | | | A6601, A6802, | | | DRB1_1101, | | | |
| | | | | A6823, A6901, | | | DRB1_1301, | | | |
| | | | | B1517, A0301, | | | DRB1_1602, | | | |
| | | | | A1101, A6801, | | | DPA10103- | | | |
| | | | | A6801, A0211, | | | DPB10201, | | | |
| | | | | B0801, A2301, | | | DPA10103- | | | |
| | | | | A2403, A3207, | | | DPB10401, | | | |
| | | | | B8301, C0702, | | | DPA10103- | | | |
| | | | | C1402, A0217, | | | DPB10402, | | | |
| | | | | C0802, C1203, | | | DPA10103- | | | |
| | | | | B3801, B1503, | | | DPB10601, | | | |
| | | | | B4801, B1509, | | | DPA10201- | | | |
| | | | | B3801, A6601, | | | DPB10101, | | | |
| | | | | B4013, A0201, | | | DPA10201- | | | |
| | | | | A0202, A0203, | | | DPB10501, | | | |
| | | | | A0205, A0207, | | | DPA10301- | | | |
| | | | | A0211, A0212, | | | DPB10402, | | | |
| | | | | A0216, A0217, | | | DQA10101- | | | |
| | | | | A0219, B3501, | | | DQB10501, | | | |
| | | | | B3503, B5301, | | | DQA10102- | | | |
| | | | | B5802, A0205, | | | DQB10602, | | | |
| | | | | A2602, A2603, | | | DQA10103- | | | |
| | | | | A0101, A2501, | | | DQB10603, | | | |
| | | | | A2601, A3002, | | | DQA10501- | | | |
| | | | | A6823, B5101, | | | DQB10201, | | | |
| | | | | C0602, C0701, | | | DQA10501- | | | |
| | | | | B7301, C0401, | | | DQB10301 | | | |
| | | | | A3201, B5801 | | | | | | |
| ORF1ab | YILFTRF | 97.6% | 70 | A1101, A2902, | 69.4% | 16 | DRB1_0101, | 0.83025507 | 0.97 | 3.47 |
| _2331 | FYVLGL | | | A3002, A3207, | | | DRB1_0402, | 8 | | |
| | AAIMQ | | | A6601, A6823, | | | DRB1_0901, | | | |
| | LFFSYF | | | A8001, B1502, | | | DRB1_1001, | | | |
| | AVHFIS | | | B4506, A0201, | | | DRB1_1602, | | | |
| | NSW | | | A0202, A0203, | | | DPA10103- | | | |
| | (SEQ ID | | | A0205, A0206, | | | DPB10201, | | | |
| | NO: 2) | | | A0211, A0212, | | | DPA10103- | | | |
| | | | | A0216, A0217, | | | DPB10301, | | | |
| | | | | A0219, A6901, | | | DPA10103- | | | |
| | | | | B0802, B4013, | | | DPB10401, | | | |
| | | | | C0602, A2403, | | | DPA10103- | | | |
| | | | | B1401, B2705, | | | DPB10601, | | | |
| | | | | B2720, B3701, | | | DPA10201- | | | |
| | | | | B3801, B3901, | | | DPB10101, | | | |
| | | | | C0701, C0702, | | | DPA10301- | | | |
| | | | | A3001, B5401, | | | DPB10402, | | | |
| | | | | B7301, C1402, | | | DQA10101- | | | |
| | | | | A2601, A2603, | | | DQB10501, | | | |
| | | | | B1517, B3501, | | | DQA10102- | | | |
| | | | | C0303, A6823, | | | DQB10502, | | | |
| | | | | B5301, B5703, | | | DQA10301- | | | |
| | | | | B5801, C0501, | | | DQB10302, | | | |
| | | | | A2501, A2602, | | | DQA10401- | | | |
| | | | | A3201, B1501, | | | DQB10201 | | | |
| | | | | B8301, A2402, | | | | | | |
| | | | | A2403, B0803, | | | | | | |
| | | | | B1503, A0207, | | | | | | |
| | | | | C0401, A0211, | | | | | | |
| | | | | A6802, B5101, | | | | | | |
| | | | | C0802, C1203, | | | | | | |
| | | | | C1502, C1203, | | | | | | |
| | | | | A3201, B1509, | | | | | | |
| | | | | A2301, B5301, | | | | | | |
| | | | | B5801, B4801 | | | | | | |
| ORF1ab | FAVHFI | 99.2% | 80 | C1203, A3201, | 55.3% | 14 | DRB1_0402, | 0.94998843 | 0.97 | 3.46 |
| _2354 | SNSWL | | | B1509, B3801, | | | DRB1_0405, | 2 | | |
| | MWLII | | | A2301, A2403, | | | DRB1_0701, | | | |
| | NLVQM | | | A2603, A2902, | | | DRB1_0901, | | | |
| | APISAM | | | B4013, C0702, | | | DRB1_1001, | | | |
| | VRMYIF | | | B5301, B5703, | | | DRB1_1602, | | | |
| | (SEQ ID | | | B5801, C0701, | | | DPA10103- | | | |
| | NO: 30 | | | C1502, A0201, | | | DPB10201, | | | |
| | | | | A0202, A0203, | | | DPA10103- | | | |
| | | | | A0211, A0212, | | | DPB10401, | | | |
| | | | | A0216, A0219, | | | DPA10103- | | | |
| | | | | A6823, A6901, | | | DPB10402, | | | |
| | | | | B3901, B4801, | | | DPA10103- | | | |
| | | | | A0207, A0211, | | | DPB10601, | | | |
| | | | | A2602, B1402, | | | DPA10201- | | | |
| | | | | B1501, B1502, | | | DPB10101, | | | |
| | | | | B1503, B2720, | | | DPA10301- | | | |
| | | | | B4506, C0303, | | | DPB10402, | | | |
| | | | | C1203, C1402, | | | DQA10101- | | | |
| | | | | A0206, A6802, | | | DQB10501, | | | |
| | | | | B0803, A6801, | | | DQA10102- | | | |
| | | | | B0702, B3501, | | | DQB10502 | | | |
| | | | | B3503, B8301, | | | | | | |
| | | | | C0401, A2501, | | | | | | |
| | | | | A8001, A6802, | | | | | | |
| | | | | B5801, A3207, | | | | | | |
| | | | | B0801, B0802, | | | | | | |
| | | | | B4601, B5701, | | | | | | |
| | | | | B5802, A2403, | | | | | | |
| | | | | A2602, A6601, | | | | | | |
| | | | | A0217, A3001, | | | | | | |
| | | | | B7301, A2402, | | | | | | |
| | | | | A3002, A3201, | | | | | | |
| | | | | A3207, B1517, | | | | | | |
| | | | | B4201, A0101, | | | | | | |
| | | | | A0301, A1101, | | | | | | |
| | | | | A2501, A2601, | | | | | | |
| | | | | A6801, C0602, | | | | | | |
| | | | | A0205, A2301, | | | | | | |
| | | | | A3101, B5301 | | | | | | |
| ORF1ab | VTCLAY | 98.4% | 82 | A2603, A6823, | 64.1% | 11 | DRB1_0402, | 0.86667279 | 0.97 | 3.46 |
| _3057 | YFMRF | | | A3101, A2403, | | | DRB1_0403, | | | |
| | RRAFGE | | | A2602, A6601, | | | DRB1_0801, | | | |
| | YSHVVA | | | B0801, B0802, | | | DRB1_0802, | | | |
| | FNTLLF | | | B1502, B1503, | | | DRB1_1101, | | | |
| | LMSF | | | B4506, A0302, | | | DPA10103- | | | |
| | (SEQ ID | | | A3301, A6801, | | | DPB10201, | | | |
| | NO: 4) | | | B1402, B7301, | | | DPA10103- | | | |
| | | | | C0602, C0701, | | | DPB10401, | | | |
| | | | | C1402, A2402, | | | DPA10103- | | | |
| | | | | A2403, B0803, | | | DPB10601, | | | |
| | | | | B1402, B4013, | | | DPA10201- | | | |
| | | | | B8301, C0702, | | | DPB10501, | | | |
| | | | | C1203, B0801, | | | DQA10102- | | | |
| | | | | B0802, B1401, | | | DQB10602, | | | |
| | | | | B2720, A3001, | | | DQA10501- | | | |
| | | | | A3002, A8001, | | | DQB10301 | | | |
| | | | | C0401, B7301, | | | | | | |
| | | | | B2720, A3207, | | | | | | |
| | | | | C1502, A0205, | | | | | | |
| | | | | A3201, B1801, | | | | | | |
| | | | | B3701, B4001, | | | | | | |
| | | | | B4002, B4402, | | | | | | |
| | | | | B4403, B4501, | | | | | | |
| | | | | B4801, B1509, | | | | | | |
| | | | | B3801, B3901, | | | | | | |
| | | | | A0217, A2603, | | | | | | |
| | | | | A6802, A6901, | | | | | | |
| | | | | A2301, A2402, | | | | | | |
| | | | | A2902, A3201, | | | | | | |
| | | | | B1517, A6823, | | | | | | |
| | | | | A2501, B5701, | | | | | | |
| | | | | A0201, A0205, | | | | | | |
| | | | | A0206, A0207, | | | | | | |
| | | | | A0211, A0212, | | | | | | |
| | | | | A0216, A0217, | | | | | | |
| | | | | A0219, B3503, | | | | | | |
| | | | | A0202, A0211, | | | | | | |
| | | | | A0302, A0203, | | | | | | |
| | | | | A2501, B4601, | | | | | | |
| | | | | B5401, C0802 | | | | | | |
| M_87 | LVGLM | 97.9% | 66 | A0101, A2501, | 70.4% | 12 | DRB1_0402, | 0.75596707 | 0.93 | 3.37 |
| | WLSYFI | | | A2902, A3201, | | | DRB1_0403, | 2 | | |
| | ASFRLF | | | A6601, A8001, | | | DRB1_1501, | | | |
| | ARTRS | | | B5703, A0201, | | | DRB1_1602, | | | |
| | MWSF | | | A0202, A0203, | | | DPA10103- | | | |
| | NPETNI | | | A0206, A0211, | | | DPB10201, | | | |
| | L | | | A0212, A0216, | | | DPA10103- | | | |
| | (SEQ ID | | | A0217, A0219, | | | DPB10401, | | | |
| | NO: 5) | | | A3207, A6823, | | | DPA10103- | | | |
| | | | | B4013, B4506, | | | DPB10601, | | | |
| | | | | B5401, B7301, | | | DPA10201- | | | |
| | | | | A0205, A2602, | | | DPB10101, | | | |
| | | | | A2603, B0802, | | | DQA10101- | | | |
| | | | | B0803, B1501, | | | DQB10501, | | | |
| | | | | B1502, B1503, | | | DQA10102- | | | |
| | | | | A0301, A0302, | | | DQB10502, | | | |
| | | | | A1101, A3101, | | | DQA10102- | | | |
| | | | | A3301, A6801, | | | DQB10602, | | | |
| | | | | A2301, A2402, | | | DQA10501- | | | |
| | | | | A2403, C0702, | | | DQB10301 | | | |
| | | | | C1402, C0401, | | | | | | |
| | | | | A0202, A6802, | | | | | | |
| | | | | A3101, B1402, | | | | | | |
| | | | | A0302, B7301, | | | | | | |
| | | | | A3201, B0801, | | | | | | |
| | | | | B2720, C0602, | | | | | | |
| | | | | C0701, C1203, | | | | | | |
| | | | | A2403, B5703, | | | | | | |
| | | | | B0802, A2602, | | | | | | |
| | | | | B1401, B2705, | | | | | | |
| | | | | A3001, A3207, | | | | | | |
| | | | | C1502, A0217, | | | | | | |
| | | | | C0802, B3901 | | | | | | |
| ORF1ab | LAHIQ | 97.4% | 75 | B3501, B3503, | 34.3% | 9 | DRB1_0301, | 0.99068893 | 0.98 | 3.29 |
| _3123 | WMVM | | | A2403, A2602, | | | DRB1_0401, | 6 | | |
| | FTPLVP | | | B1509, B3701, | | | DRB1_0404, | | | |
| | FWITIA | | | B3801, B7301, | | | DRB1_0405, | | | |
| | YIICISTK | | | A2301, A2402, | | | DRB1_0802, | | | |
| | HFYW | | | A2403, A3207, | | | DRB1_1602, | | | |
| | (SEQ ID | | | A6601, A6823, | | | DPA10103- | | | |
| | NO: 6) | | | B2720, B4013, | | | DPB10201, | | | |
| | | | | B4801, C0401, | | | DPA10103- | | | |
| | | | | C1402, A0201, | | | DPB10401, | | | |
| | | | | A0202, A0203, | | | DPA10103- | | | |
| | | | | A0205, A0206, | | | DPB10601 | | | |
| | | | | A0207, A0211, | | | | | | |
| | | | | A0212, A0216, | | | | | | |
| | | | | A0217, A0219, | | | | | | |
| | | | | A6901, A0211, | | | | | | |
| | | | | A3201, B1501, | | | | | | |
| | | | | B1502, B1503, | | | | | | |
| | | | | B4601, C0702, | | | | | | |
| | | | | C1203, B5301, | | | | | | |
| | | | | B5703, B5801, | | | | | | |
| | | | | B5802, A0217, | | | | | | |
| | | | | A6802, C0501, | | | | | | |
| | | | | B5401, A3201, | | | | | | |
| | | | | A3002, A8001, | | | | | | |
| | | | | B1801, B3501, | | | | | | |
| | | | | B4201, B4506, | | | | | | |
| | | | | B8301, B1517, | | | | | | |
| | | | | C1502, B0802, | | | | | | |
| | | | | A2602, B0802, | | | | | | |
| | | | | A2402, A2601, | | | | | | |
| | | | | A2603, A3001, | | | | | | |
| | | | | B0803, A3207, | | | | | | |
| | | | | B1401, B7301, | | | | | | |
| | | | | A2501, A2902, | | | | | | |
| | | | | A3002, C0602, | | | | | | |
| | | | | C0701, A6801, | | | | | | |
| | | | | A3301 | | | | | | |
| ORF1ab | TVVIGT | 96.6% | 53 | A2501, A2601, | 62.5% | 10 | DRB1_0101, | 0.71712749 | 0.96 | 3.26 |
| _4978 | SKFYGG | | | A2602, B1502, | | | DRB1_0404, | 4 | | |
| | WHNM | | | A2601, A2603, | | | DRB1_0701, | | | |
| | LKTVYS | | | A2902, A3002, | | | DRB1_0802, | | | |
| | DVENP | | | A6601, A8001, | | | DRB1_0901, | | | |
| | HLMG | | | A2603, B1517, | | | DRB1_1301, | | | |
| | WD | | | B5701, B5703, | | | DRB1_1602, | | | |
| | (SEQ ID | | | B7301, A3207, | | | DPA10103- | | | |
| | NO: 7) | | | B2720, B4013, | | | DPB10301, | | | |
| | | | | C1402, A2301, | | | DPA10103- | | | |
| | | | | A2402, A2403, | | | DQA10501- | | | |
| | | | | B3901, C0401, | | | DQB10301 | | | |
| | | | | C0602, C0701, | | | | | | |
| | | | | C0702, B1801, | | | | | | |
| | | | | B3801, A0203, | | | | | | |
| | | | | A0205, B0801, | | | | | | |
| | | | | B0803, C0501, | | | | | | |
| | | | | C0802, B4402, | | | | | | |
| | | | | B4403, A8001, | | | | | | |
| | | | | B1801, B3501, | | | | | | |
| | | | | B8301, A0301, | | | | | | |
| | | | | A1101, A3101, | | | | | | |
| | | | | A3201, A3207, | | | | | | |
| | | | | A3301, A6801, | | | | | | |
| | | | | A6823, B4506, | | | | | | |
| | | | | A0216, A0302, | | | | | | |
| | | | | C1402 | | | | | | |
| ORF1ab | FRLTLG | 96.6% | 47 | B7301, A2902, | 54.5% | 10 | DRB1_0801, | 0.70581673 | 0.96 | 3.18 |
| _3804 | VYDYLV | | | A3002, A8001, | | | DRB1_1001, | 4 | | |
| | STQEFR | | | A0211, A0216, | | | DRB1_1301, | | | |
| | YMNSQ | | | A2403, A6823, | | | DPA10103- | | | |
| | GLLPPK | | | A6901, A0201, | | | DPB10201, | | | |
| | NSID | | | A0202, A0212, | | | DPA10103- | | | |
| | (SEQ ID | | | A0217, A0219, | | | DPB10301, | | | |
| | NO: 8) | | | A0206, A2301, | | | DPA10103- | | | |
| | | | | A2402, C1402, | | | DPB10401, | | | |
| | | | | A6801, A0101, | | | DPA10103- | | | |
| | | | | A2902, B3501, | | | DPB10601, | | | |
| | | | | C0602, C1502, | | | DPA10301- | | | |
| | | | | B1401, B1402, | | | DPB10402, | | | |
| | | | | B1502, B1509, | | | DQA10301- | | | |
| | | | | B2705, B2720, | | | DQB10302, | | | |
| | | | | B3901, B4013, | | | DQA10401- | | | |
| | | | | B4801, B7301, | | | DQB10402 | | | |
| | | | | C0303, C0602, | | | | | | |
| | | | | C0701, C0702, | | | | | | |
| | | | | A2402, A3207, | | | | | | |
| | | | | C0401, B0803, | | | | | | |
| | | | | A1101, A0212, | | | | | | |
| | | | | B5401, B8301, | | | | | | |
| | | | | A6901 | | | | | | |
| ORF1ab | YCFLGY | 96.6% | 52 | A2403, A0302, | 43.9% | 9 | DRB1_0401, | 0.82265489 | 0.95 | 3.18 |
| _3783 | FCTCYF | | | A2902, A6823, | | | DRB1_0402, | 9 | | |
| | GLFCLL | | | A8001, B1502, | | | DRB1_0403, | | | |
| | NRYFRL | | | B4506, A6601, | | | DRB1_1302, | | | |
| | TLGVYD | | | B1503, B1517, | | | DPA10103- | | | |
| | YLV | | | A2301, A2403, | | | DPB10201, | | | |
| | (SEQ ID | | | B0802, B4013, | | | DPA10103- | | | |
| | NO: 9) | | | C0702, A2602, | | | DPB10401, | | | |
| | | | | A2603, A6823, | | | DPA10103- | | | |
| | | | | B1401, B1402, | | | DPB10402, | | | |
| | | | | A2402, A3207, | | | DPA10103- | | | |
| | | | | B4801, C0701, | | | DPB10601, | | | |
| | | | | C1402, A3301, | | | DQA10102- | | | |
| | | | | A2902, A3002, | | | DQB10602 | | | |
| | | | | A3301, A0211, | | | | | | |
| | | | | A0212, A0216, | | | | | | |
| | | | | A0217, A0219, | | | | | | |
| | | | | B0801, B0801, | | | | | | |
| | | | | B1401, B7301, | | | | | | |
| | | | | A2402, B2720, | | | | | | |
| | | | | A2501, C0401, | | | | | | |
| | | | | B7301, A6901, | | | | | | |
| | | | | A0201, A0202, | | | | | | |
| | | | | A0206, A6801, | | | | | | |
| | | | | A0101, B3501, | | | | | | |
| | | | | C0602, C1502 | | | | | | |
| ORF1ab | MMILS | 96.4% | 47 | A0201, A0202, | 60.8% | 21 | DRB1_0101, | 0.65006591 | 0.95 | 3.17 |
| _5147 | DDAW | | | A0206, A0211, | | | DRB1_0301, | 6 | | |
| | CFNSTY | | | A0212, A0216, | | | DRB1_0402, | | | |
| | ASQGL | | | A0219, A0216, | | | DRB1_0403, | | | |
| | VASIKN | | | A0219, A0101, | | | DRB1_0405, | | | |
| | FKSVLY | | | C0501, C0802, | | | DRB1_0701, | | | |
| | (SEQ ID | | | A2501, A2601, | | | DRB1_0801, | | | |
| | NO: 10) | | | A2602, A2603, | | | DRB1_0802, | | | |
| | | | | A2902, A8001, | | | DRB1_1001, | | | |
| | | | | B1502, B1517, | | | DRB1_1101, | | | |
| | | | | B3501, B1509, | | | DRB1_1301, | | | |
| | | | | A6802, A6901, | | | DRB1_1602, | | | |
| | | | | C1502, B3501, | | | DPA10103- | | | |
| | | | | C1203, A3201, | | | DPB10201, | | | |
| | | | | B1502, A0301, | | | DPA10103- | | | |
| | | | | A1101, A6801, | | | DPB10301, | | | |
| | | | | C1502, A8001, | | | DPA10103- | | | |
| | | | | A6601, A6823, | | | DPB10401, | | | |
| | | | | B1503, A3301, | | | DPA10201- | | | |
| | | | | A6901, B1501, | | | DPB10101, | | | |
| | | | | C1402, A2301, | | | DPA10201- | | | |
| | | | | A2403, C0602, | | | DPB10501, | | | |
| | | | | C0701, C0702, | | | DPA10201- | | | |
| | | | | A2403 | | | DPB11401, | | | |
| | | | | | | | DPA10301- | | | |
| | | | | | | | DPB10402, | | | |
| | | | | | | | DQA10102- | | | |
| | | | | | | | DQB10502, | | | |
| | | | | | | | DQA10102- | | | |
| | | | | | | | DQB10602 | | | |
| ORF3a_ | NFVRII | 98.0% | 63 | A2301, B1401, | 46.5% | 15 | DRB1_0103, | 0.80377466 | 0.91 | 3.15 |
| 119 | MRLWL | | | A2501, B5701, | | | DRB1_0404, | | | |
| | CWKCR | | | B5703, B7301, | | | DRB1_0405, | | | |
| | SKNPLL | | | A2402, A3201, | | | DRB1_0801, | | | |
| | YDANYF | | | B5301, B5801, | | | DRB1_1001, | | | |
| | LCWHT | | | A0301, A0302, | | | DRB1_1201, | | | |
| | (SEQ ID | | | A3001, A3101, | | | DRB1_1301, | | | |
| | NO: 11) | | | A0302, C0401, | | | DRB1_1501, | | | |
| | | | | B1509, B2720, | | | DRB1_1602, | | | |
| | | | | B3901, B4801, | | | DPA10103- | | | |
| | | | | A3001, A8001, | | | DPB10201, | | | |
| | | | | C0602, C0701, | | | DPA10103- | | | |
| | | | | B8301, A0201, | | | DPB10401, | | | |
| | | | | A0202, A0203, | | | DPA10103- | | | |
| | | | | A0206, A0211, | | | DPB10402, | | | |
| | | | | A0212, A0216, | | | DPA10103- | | | |
| | | | | A0217, A0219, | | | DPB10601, | | | |
| | | | | A6601, A6823, | | | DPA10201- | | | |
| | | | | A6901, B0803, | | | DPB10101, | | | |
| | | | | B4013, B4506, | | | DPA10301- | | | |
| | | | | C0303, C0501, | | | DPB10402 | | | |
| | | | | A2403, A6823, | | | | | | |
| | | | | B3701, B4403, | | | | | | |
| | | | | A0101, A2501, | | | | | | |
| | | | | A2902, A8001, | | | | | | |
| | | | | B0802, B1502, | | | | | | |
| | | | | B3501, C1203, | | | | | | |
| | | | | A2902, A3002, | | | | | | |
| | | | | B3801, A0212, | | | | | | |
| | | | | A2603, A6802, | | | | | | |
| | | | | C1502 | | | | | | |
| ORF1ab | RLYLDA | 99.2% | 73 | A0212, A0219, | 48.4% | 12 | DRB1_0401, | 0.70195600 | 0.95 | 3.13 |
| _6417 | YNMMI | | | B1503, B2720, | | | DRB1_0403, | 8 | | |
| | SAGFSL | | | B4013, C1402, | | | DRB1_0405, | | | |
| | WVYKQ | | | A2402, A2403, | | | DRB1_0701, | | | |
| | FDTYNL | | | C0602, A0101, | | | DRB1_0901, | | | |
| | WNTFT | | | A0201, A0202, | | | DRB1_1001, | | | |
| | (SEQ ID | | | A0205, A0206, | | | DRB1_1602, | | | |
| | NO: 12) | | | A0207, A0211, | | | DPA10103- | | | |
| | | | | A0212, A0216, | | | DPB10301, | | | |
| | | | | A0217, A6901, | | | DPA10201- | | | |
| | | | | B0802, B0803, | | | DPB11401, | | | |
| | | | | C0401, C0501, | | | DQA10101- | | | |
| | | | | C0802, C1203, | | | DQB10501, | | | |
| | | | | B1401, A3207, | | | DQA10102- | | | |
| | | | | A6823, B1502, | | | DQB10502, | | | |
| | | | | B8301, A0201, | | | DQA10102- | | | |
| | | | | A0203, A3201, | | | DQB10602 | | | |
| | | | | A3207, A6601, | | | | | | |
| | | | | B1501, B1509, | | | | | | |
| | | | | B3801, B3901, | | | | | | |
| | | | | B4506, B4801, | | | | | | |
| | | | | C0303, B1517, | | | | | | |
| | | | | B5301, B5801, | | | | | | |
| | | | | C1502, A3002, | | | | | | |
| | | | | A8001, A0301, | | | | | | |
| | | | | A1101, A3001, | | | | | | |
| | | | | A3101, B1517, | | | | | | |
| | | | | B5703, B5802, | | | | | | |
| | | | | A2501, A2601, | | | | | | |
| | | | | A2602, A2603, | | | | | | |
| | | | | A2902, B3501, | | | | | | |
| | | | | A6823, A2301, | | | | | | |
| | | | | B5701, B1402, | | | | | | |
| | | | | A3301, A6801, | | | | | | |
| | | | | B0801, B1401, | | | | | | |
| | | | | C0701, C1502, | | | | | | |
| | | | | C0602 | | | | | | |
| ORF1ab | QQESPF | 94.8% | 51 | B1503, B4801, | 54.7% | 9 | DRB1_0101, | 0.63990840 | 0.96 | 3.09 |
| _1799 | VMMS | | | B4501, A2501, | | | DRB1_0404, | 3 | | |
| | APPAQ | | | A6802, B5401, | | | DRB1_0405, | | | |
| | YELKHG | | | B8301, A0201, | | | DRB1_0802, | | | |
| | TFTCAS | | | A0206, A0216, | | | DRB1_0901, | | | |
| | EYTGNY | | | A0217, A0219, | | | DRB1_1001, | | | |
| | (SEQ ID | | | A6901, B3501, | | | DRB1_1602, | | | |
| | NO: 13) | | | B4506, B5401, | | | DQA10103- | | | |
| | | | | B7301, C1402, | | | DQB10603, | | | |
| | | | | A3002, A6823, | | | DQA10501- | | | |
| | | | | A8001, B0802, | | | DQB10301 | | | |
| | | | | B0803, B1501, | | | | | | |
| | | | | B1502, B1503, | | | | | | |
| | | | | B1517, B5801, | | | | | | |
| | | | | A6823, C0303, | | | | | | |
| | | | | A2403, C0702, | | | | | | |
| | | | | B4002, A2902, | | | | | | |
| | | | | B4601, A0101, | | | | | | |
| | | | | A2601, A2602, | | | | | | |
| | | | | A2603, C1203, | | | | | | |
| | | | | B4501, C0401, | | | | | | |
| | | | | A2603, A3002, | | | | | | |
| | | | | A0211, B2720, | | | | | | |
| | | | | B3701, B4013, | | | | | | |
| | | | | C0701 | | | | | | |
| ORF1ab | DVLLPL | 96.6% | 60 | A2501, A2602, | 36.5% | 10 | DRB1_0101, | 0.77927383 | 0.98 | 3.09 |
| _3196 | TQYNR | | | B3501, B5101, | | | DRB1_0301, | 5 | | |
| | YLALYN | | | B5301, B8301, | | | DRB1_0405, | | | |
| | KYKYFS | | | C0602, A3207, | | | DRB1_1501, | | | |
| | GAMDT | | | B0801, B0802, | | | DRB1_1602, | | | |
| | TSYRE | | | B0803, B1401, | | | DPA10103- | | | |
| | (SEQ ID | | | B1402, B1502, | | | DPB10401, | | | |
| | NO: 14) | | | B1503, B1509, | | | DPA10201- | | | |
| | | | | B2720, B3701, | | | DPB10101, | | | |
| | | | | B3901, B4013, | | | DPA10201- | | | |
| | | | | B4506, B4801, | | | DPB10501, | | | |
| | | | | C0303, C0401, | | | DPA10201- | | | |
| | | | | C0702, C1402, | | | DPB11401, | | | |
| | | | | A2602, A2902, | | | DPA10301- | | | |
| | | | | A3002, A8001, | | | DPB10402 | | | |
| | | | | B1401, B2705, | | | | | | |
| | | | | B7301, A2301, | | | | | | |
| | | | | A2402, A2403, | | | | | | |
| | | | | A3207, B0802, | | | | | | |
| | | | | A2902, A6601, | | | | | | |
| | | | | B1517, B3501, | | | | | | |
| | | | | B5701, B0801, | | | | | | |
| | | | | B7301, A3001, | | | | | | |
| | | | | A3002, A1101, | | | | | | |
| | | | | A3101, A6801, | | | | | | |
| | | | | A6823, A0302, | | | | | | |
| | | | | C1502, B0803, | | | | | | |
| | | | | B3801, C0602, | | | | | | |
| | | | | C0701, B4002, | | | | | | |
| | | | | B4501, A6801 | | | | | | |
| ORF1ab | RSQMEI | 90.5% | 32 | C1502, B4801, | 59.4% | 8 | DRB1_0405, | 0.61488341 | 0.95 | 3.07 |
| _6658 | DFLELA | | | B0803, B1801, | | | DPA10103- | 1 | | |
| | MDEFIE | | | B4403, B4501, | | | DPB10402, | | | |
| | RYKLEG | | | B4002, A6801, | | | DPA10103- | | | |
| | YAFEHI | | | A0101, A8001, | | | DPB10601, | | | |
| | VYG | | | A0302, B1401, | | | DQA10101- | | | |
| | (SEQ ID | | | B1801, B7301, | | | DQB10501, | | | |
| | NO: 15) | | | A2301, A2402, | | | DQA10102- | | | |
| | | | | A2403, A3207, | | | DQB10502, | | | |
| | | | | B0802, B0803, | | | DQA10301- | | | |
| | | | | B1503, C0702, | | | DQB10302, | | | |
| | | | | C1402, A6823, | | | DQA10401- | | | |
| | | | | C0602, C0701, | | | DQB10402, | | | |
| | | | | C1203, B4001, | | | DQA10501- | | | |
| | | | | A2603, C0303, | | | DQB10201 | | | |
| | | | | C0501, C0802 | | | | | | |
| ORF1ab | DDTLRV | 96.9% | 57 | B0802, B1402, | 38.1% | 3 | DRB1_0405, | 0.78800084 | 0.90 | 3.04 |
| _1624 | EAFEYY | | | A2902, A8001, | | | DQA10301- | 9 | | |
| | HTTDPS | | | C0602, C0701, | | | DQB10302, | | | |
| | FLGRY | | | A2501, A6823, | | | DQA10401- | | | |
| | MSALN | | | A6901, B1401, | | | DQB10402 | | | |
| | HTKKW | | | C1203, B7301, | | | | | | |
| | (SEQ ID | | | A6601, C1402, | | | | | | |
| | NO: 16) | | | A2301, A2402, | | | | | | |
| | | | | A2403, A3207, | | | | | | |
| | | | | B1502, B1503, | | | | | | |
| | | | | B4013, C0401, | | | | | | |
| | | | | C0702, B1509, | | | | | | |
| | | | | B3801, B3901, | | | | | | |
| | | | | A1101, A2603, | | | | | | |
| | | | | B4201, B8301, | | | | | | |
| | | | | A0201, A0202, | | | | | | |
| | | | | A0203, A0205, | | | | | | |
| | | | | A0207, A0211, | | | | | | |
| | | | | A0212, A0216, | | | | | | |
| | | | | A0217, A0219, | | | | | | |
| | | | | A2602, B0801, | | | | | | |
| | | | | B0802, B0803, | | | | | | |
| | | | | B2720, C0303, | | | | | | |
| | | | | C0802, A2402, | | | | | | |
| | | | | A6801, B4506, | | | | | | |
| | | | | A6801, A3207, | | | | | | |
| | | | | B5701, B5703, | | | | | | |
| | | | | A0301, B1503, | | | | | | |
| | | | | B4801 | | | | | | |
| S_882 | ITSGWT | 96.0% | 60 | A6802, A6901, | 76.9% | 11 | DRB1_0101, | 0.50567056 | 0.80 | 3.03 |
| | FGAGA | | | A2602, A2603, | | | DRB1_0402, | 9 | | |
| | ALQIPF | | | A6802, A6823, | | | DRB1_0404, | | | |
| | AMQM | | | B1502, B1509, | | | DRB1_0701, | | | |
| | AYRFN | | | B1517, B3801, | | | DRB1_0901, | | | |
| | GIGVTQ | | | B3901, B4013, | | | DRB1_1501, | | | |
| | N | | | B4801, C0303, | | | DQA10102- | | | |
| | (SEQ ID | | | C0401, C1203, | | | DQB10602, | | | |
| | NO: 17) | | | C1402, C1502, | | | DQA10103- | | | |
| | | | | C0401, A3207, | | | DQB10603, | | | |
| | | | | B3501, B4601, | | | DQA10301- | | | |
| | | | | A0206, B1501, | | | DQB10302, | | | |
| | | | | B1503, B2720, | | | DQA10401- | | | |
| | | | | B4506, A2902, | | | DQB10402, | | | |
| | | | | A8001, B1801, | | | DQA10501- | | | |
| | | | | B3503, B5101, | | | DQB10301 | | | |
| | | | | B5301, B8101, | | | | | | |
| | | | | B8301, A3301, | | | | | | |
| | | | | A2301, A2402, | | | | | | |
| | | | | A2403, A2902, | | | | | | |
| | | | | A3201, A3207, | | | | | | |
| | | | | A6601, B0801, | | | | | | |
| | | | | B0802, B0803, | | | | | | |
| | | | | B5703, B5801, | | | | | | |
| | | | | B5802, C0501, | | | | | | |
| | | | | C0602, C0702, | | | | | | |
| | | | | B4013, A0203, | | | | | | |
| | | | | A0212, B1402, | | | | | | |
| | | | | A3001, B2720, | | | | | | |
| | | | | B7301, C0701 | | | | | | |
| ORF1ab | SQLMC | 95.2% | 47 | B1509, B3801, | 54.0% | 3 | DPA10103- | 0.55421006 | 0.96 | 3.00 |
| _2562 | QPILLL | | | B3901, B4013, | | | DPB10402, | 6 | | |
| | DQALV | | | B4801, C0401, | | | DQA10102- | | | |
| | SDVGD | | | A0201, A0202, | | | DQB10602, | | | |
| | SAEVAV | | | A0219, B0803, | | | DQA10501- | | | |
| | KMFDA | | | A0211, A0217, | | | DQB10301 | | | |
| | Y | | | B5401, A0205, | | | | | | |
| | (SEQ ID | | | A0212, A0216, | | | | | | |
| | NO: 18) | | | C0501, C0802, | | | | | | |
| | | | | A2601, C1502, | | | | | | |
| | | | | B5802, B3501, | | | | | | |
| | | | | B4601, A0205, | | | | | | |
| | | | | A3001, A0211, | | | | | | |
| | | | | A2301, A2403, | | | | | | |
| | | | | C0501, C0702, | | | | | | |
| | | | | C1402, B1402, | | | | | | |
| | | | | C1402, A2501, | | | | | | |
| | | | | A2601, A2603, | | | | | | |
| | | | | A3201, A6601, | | | | | | |
| | | | | A6823, B1501, | | | | | | |
| | | | | B1502, B1503, | | | | | | |
| | | | | B3501, B5301, | | | | | | |
| | | | | C0303, C0701, | | | | | | |
| | | | | C1203 | | | | | | |
| ORF1ab | SLVLAR | 96.6% | 55 | B1402, B0801, | 41.6% | 11 | DRB1_0101, | 0.66114165 | 0.95 | 2.99 |
| _5027 | KHTTCC | | | A0302, B1503, | | | DRB1_0301, | 2 | | |
| | SLSHRF | | | B1509, B2720, | | | DRB1_0403, | | | |
| | YRLANE | | | B3901, B4201, | | | DRB1_0404, | | | |
| | CAQVLS | | | B4801, C1402, | | | DRB1_0801, | | | |
| | EMV | | | A2603, A2603, | | | DRB1_1101, | | | |
| | (SEQ ID | | | A3101, A3301, | | | DRB1_1301, | | | |
| | NO: 19) | | | A6801, A1101, | | | DRB1_1501, | | | |
| | | | | A0202, A0212, | | | DRB1_1602, | | | |
| | | | | B0801, B0802, | | | DPA10103- | | | |
| | | | | A3001, B1401, | | | DPB10301, | | | |
| | | | | C0702, A0203, | | | DQA10102- | | | |
| | | | | A0205, A0211, | | | DQB10602 | | | |
| | | | | A0216, A0219, | | | | | | |
| | | | | B3501, C0303, | | | | | | |
| | | | | C0501, C0602, | | | | | | |
| | | | | C0802, C1203, | | | | | | |
| | | | | C1502, A2501, | | | | | | |
| | | | | A2602, A6601, | | | | | | |
| | | | | B4501, A0301, | | | | | | |
| | | | | A1101, A2601, | | | | | | |
| | | | | A2902, A3002, | | | | | | |
| | | | | A3207, A6823, | | | | | | |
| | | | | A8001, B1501, | | | | | | |
| | | | | B1502, B1517, | | | | | | |
| | | | | B3501, B4506, | | | | | | |
| | | | | A0201, A0202, | | | | | | |
| | | | | A0206 | | | | | | |
| ORF1ab | LVLARK | 96.6% | 55 | B1402, B0801, | 41.6% | 11 | DRB1_0101, | 0.66114165 | 0.95 | 2.99 |
| _5028 | HTTCCS | | | A0302, B1503, | | | DRB1_0301, | 2 | | |
| | LSHRFY | | | B1509, B2720, | | | DRB1_0403, | | | |
| | RLANEC | | | B3901, B4201, | | | DRB1_0404, | | | |
| | AQVLSE | | | B4801, C1402, | | | DRB1_0801, | | | |
| | MVM | | | A2603, A2603, | | | DRB1_1101, | | | |
| | (SEQ ID | | | A3101, A3301, | | | DRB1_1301, | | | |
| | NO: 20) | | | A6801, A1101, | | | DRB1_1501, | | | |
| | | | | A0202, A0212, | | | DRB1_1602, | | | |
| | | | | B0801, B0802, | | | DPA10103- | | | |
| | | | | A3001, B1401, | | | DPB10301, | | | |
| | | | | C0702, A0203, | | | DQA10102- | | | |
| | | | | A0205, A0211, | | | DQB10602 | | | |
| | | | | A0216, A0219, | | | | | | |
| | | | | B3501, C0303, | | | | | | |
| | | | | C0501, C0602, | | | | | | |
| | | | | C0802, C1203, | | | | | | |
| | | | | C1502, A2501, | | | | | | |
| | | | | A2602, A6601, | | | | | | |
| | | | | B4501, A0301, | | | | | | |
| | | | | A1101, A2601, | | | | | | |
| | | | | A2902, A3002, | | | | | | |
| | | | | A3207, A6823, | | | | | | |
| | | | | A8001, B1501, | | | | | | |
| | | | | B1502, B1517, | | | | | | |
| | | | | B3501, B4506, | | | | | | |
| | | | | A0201, A0202, | | | | | | |
| | | | | A0206 | | | | | | |
| ORF1ab | VLARKH | 96.6% | 55 | B1402, B0801, | 41.6% | 11 | DRB1_0101, | 0.66114165 | 0.95 | 2.99 |
| _5029 | TTCCSL | | | A0302, B1503, | | | DRB1_0301, | 2 | | |
| | SHRFYR | | | B1509, B2720, | | | DRB1_0403, | | | |
| | LANECA | | | B3901, B4201, | | | DRB1_0404, | | | |
| | QVLSE | | | B4801, C1402, | | | DRB1_0801, | | | |
| | MVMC | | | A2603, A2603, | | | DRB1_1101, | | | |
| | (SEQ ID | | | A3101, A3301, | | | DRB1_1301, | | | |
| | NO: 21) | | | A6801, A1101, | | | DRB1_1501, | | | |
| | | | | A0202, A0212, | | | DRB1_1602, | | | |
| | | | | B0801, B0802, | | | DPA10103- | | | |
| | | | | A3001, B1401, | | | DPB10301, | | | |
| | | | | C0702, A0203, | | | DQA10102- | | | |
| | | | | A0205, A0211, | | | DQB10602 | | | |
| | | | | A0216, A0219, | | | | | | |
| | | | | B3501, C0303, | | | | | | |
| | | | | C0501, C0602, | | | | | | |
| | | | | C0802, C1203, | | | | | | |
| | | | | C1502, A2501, | | | | | | |
| | | | | A2602, A6601, | | | | | | |
| | | | | B4501, A0301, | | | | | | |
| | | | | A1101, A2601, | | | | | | |
| | | | | A2902, A3002, | | | | | | |
| | | | | A3207, A6823, | | | | | | |
| | | | | A8001, B1501, | | | | | | |
| | | | | B1502, B1517, | | | | | | |
| | | | | B3501, B4506, | | | | | | |
| | | | | A0201, A0202, | | | | | | |
| | | | | A0206 | | | | | | |
| ORF1ab | MTYRR | 93.1% | 54 | A2501, A2601, | 37.1% | 11 | DRB1_0101, | 0.72716460 | 0.96 | 2.99 |
| _5974 | LISMM | | | A2602, A2603, | | | DRB1_0103, | 3 | | |
| | GFKMN | | | A3001, A3201, | | | DRB1_0401, | | | |
| | YQVNG | | | A6601, A6823, | | | DRB1_0404, | | | |
| | YPNMFI | | | A6901, B0801, | | | DRB1_0801, | | | |
| | TREEAI | | | B0802, B1401, | | | DRB1_1001, | | | |
| | R | | | B1402, B1517, | | | DRB1_1101, | | | |
| | (SEQ ID | | | B4506, B4601, | | | DRB1_1501, | | | |
| | NO: 22) | | | B5701, B5801, | | | DRB1_1602, | | | |
| | | | | B8301, C0303, | | | DPA10103- | | | |
| | | | | C0602, C0701, | | | DPB10301, | | | |
| | | | | C1203, C1402, | | | DQA10101- | | | |
| | | | | C1502, A2403, | | | DQB10501 | | | |
| | | | | C0702, B7301, | | | | | | |
| | | | | A3207, B2705, | | | | | | |
| | | | | B2720, A0301, | | | | | | |
| | | | | A1101, A2902, | | | | | | |
| | | | | A3002, A3207, | | | | | | |
| | | | | A8001, B1502, | | | | | | |
| | | | | B1503, B4013, | | | | | | |
| | | | | A0302, B5401, | | | | | | |
| | | | | A3002, A0206, | | | | | | |
| | | | | B1509, B4801, | | | | | | |
| | | | | B8301, B0802, | | | | | | |
| | | | | A6801, C0602, | | | | | | |
| | | | | B4501, B5703, | | | | | | |
| | | | | A3001, A3201 | | | | | | |
| ORF1ab | QVVDA | 90.8% | 29 | A6601, C0501, | 59.4% | 8 | DRB1_0301, | 0.51181479 | 0.97 | 2.98 |
| _4100 | DSKIVQ | | | C0802, B1401, | | | DPA10103- | 1 | | |
| | LSEISM | | | B1509, B1517, | | | DPB10401, | | | |
| | DNSPN | | | C0303, C1502, | | | DPA10301- | | | |
| | LAWPLI | | | A0101, B4402, | | | DPB10402, | | | |
| | VTALR | | | B4403, B5301, | | | DQA10101- | | | |
| | (SEQ ID | | | B4013, B3503, | | | DQB10501, | | | |
| | NO: 23) | | | B5101, B8301, | | | DQA10102- | | | |
| | | | | B3701, B5401, | | | DQB10502, | | | |
| | | | | A2403, B1402, | | | DQA10103- | | | |
| | | | | C0401, C1402, | | | DQB10603, | | | |
| | | | | C0401, B1402, | | | DQA10301- | | | |
| | | | | A6901, A0301, | | | DQB10302, | | | |
| | | | | A3001, B2705, | | | DQA10401- | | | |
| | | | | C0602 | | | DQB10402 | | | |
| ORF1ab | GVPVV | 95.0% | 38 | A6823, A2501, | 61.5% | 6 | DRB1_0901, | 0.45356050 | 0.95 | 2.97 |
| _4622 | DSYYSL | | | A2603, B3503, | | | DRB1_1001, | 6 | | |
| | LMPILT | | | C0303, C0401, | | | DPA10103- | | | |
| | LTRALT | | | C0501, C0701, | | | DPB10201, | | | |
| | AESHV | | | C0802, B3701, | | | DPA10103- | | | |
| | DTDLT | | | A2301, A2402, | | | DPB10401, | | | |
| | (SEQ ID | | | A2403, A3201, | | | DPA10103- | | | |
| | NO: 24) | | | A3207, B2720, | | | DPB10402, | | | |
| | | | | B4013, B4506, | | | DQA10501- | | | |
| | | | | C0702, C1402, | | | DQB10301 | | | |
| | | | | A2402, C0401, | | | | | | |
| | | | | A0211, A0216, | | | | | | |
| | | | | A0217, A0201, | | | | | | |
| | | | | A0202, A0203, | | | | | | |
| | | | | A0206, A0219, | | | | | | |
| | | | | B5101, B5401, | | | | | | |
| | | | | B8301, C1502, | | | | | | |
| | | | | B4001, B4403, | | | | | | |
| | | | | B1509, A6901 | | | | | | |
| ORF1ab | AYPLTK | 96.8% | 55 | A6601, B1502, | 44.9% | 8 | DRB1_0701, | 0.62739217 | 0.93 | 2.97 |
| _5258 | HPNQE | | | B1503, B1517, | | | DPA10103- | 2 | | |
| | YADVF | | | B3503, B8301, | | | DPB10301, | | | |
| | HLYLQY | | | B4001, B4002, | | | DPA10103- | | | |
| | IRKLHD | | | B4402, B4403, | | | DPB10601, | | | |
| | ELTGH | | | B4501, B4801, | | | DPA10201- | | | |
| | (SEQ ID | | | C0401, A2501, | | | DPB11401, | | | |
| | NO: 25) | | | A2601, A2602, | | | DPA10301- | | | |
| | | | | A2902, A3002, | | | DPB10402, | | | |
| | | | | A6601, A0101, | | | DQA10101- | | | |
| | | | | A6823, C0303, | | | DQB10501, | | | |
| | | | | C0501, C0802, | | | DQA10401- | | | |
| | | | | C1203, C1502, | | | DQB10402, | | | |
| | | | | A2603, A8001, | | | DQA10501- | | | |
| | | | | A2403, C0602, | | | DQB10201 | | | |
| | | | | C0701, B1509, | | | | | | |
| | | | | A0301, A0302, | | | | | | |
| | | | | A1101, A3001, | | | | | | |
| | | | | A2301, A2403, | | | | | | |
| | | | | C0702, C1402, | | | | | | |
| | | | | B0803, B1801, | | | | | | |
| | | | | A2501, B3801, | | | | | | |
| | | | | B3901, A0211, | | | | | | |
| | | | | A0219, A3207, | | | | | | |
| | | | | B1501, B1509, | | | | | | |
| | | | | B2720, B3501, | | | | | | |
| | | | | B4013, B4506, | | | | | | |
| | | | | B4601 | | | | | | |
| ORF1ab | GVLMS | 91.0% | 41 | A2603, A0101, | 61.6% | 10 | DRB1_0402, | 0.47693405 | 0.95 | 2.95 |
| _2251 | NLGMP | | | A2501, A2902, | | | DRB1_0403, | 5 | | |
| | SYCTGY | | | A3002, A6823, | | | DRB1_0404, | | | |
| | REGYLN | | | A8001, B1517, | | | DRB1_0701, | | | |
| | STNVTI | | | B3501, B4601, | | | DRB1_0901, | | | |
| | ATYCT | | | B5801, A0219, | | | DRB1_1201, | | | |
| | (SEQ ID | | | A3002, B1502, | | | DPA10201- | | | |
| | NO: 26) | | | B4506, A0302, | | | DPB11401, | | | |
| | | | | A3301, B8301, | | | DQA10102- | | | |
| | | | | C0401, A6823, | | | DQB10602, | | | |
| | | | | C0602, C0701, | | | DQA10103- | | | |
| | | | | A0201, A0202, | | | DQB10603, | | | |
| | | | | A0203, A0207, | | | DQA10501- | | | |
| | | | | A0211, A0212, | | | DQB10301 | | | |
| | | | | A0216, A0217, | | | | | | |
| | | | | A0219, A3201, | | | | | | |
| | | | | A6901, B0803, | | | | | | |
| | | | | B3901, C0501, | | | | | | |
| | | | | A2601, A2602, | | | | | | |
| | | | | A2603, C1203, | | | | | | |
| | | | | A0211 | | | | | | |
| ORF1ab | YFVKIG | 89.7% | 42 | A0302, B1402, | 40.4% | 10 | DRB1_0101, | 0.66820875 | 0.95 | 2.92 |
| _6122 | PERTCC | | | A3101, B0802, | | | DRB1_0402, | 7 | | |
| | LCDRRA | | | B1402, C0501, | | | DRB1_0701, | | | |
| | TCFSTA | | | C0802, B1401, | | | DRB1_0801, | | | |
| | SDTYAC | | | B2705, B2720, | | | DRB1_1001, | | | |
| | WHH | | | C0401, B5301, | | | DRB1_1201, | | | |
| | (SEQ ID | | | B5703, B5801, | | | DPA10103- | | | |
| | NO: 27) | | | C0303, C0501, | | | DPB10402, | | | |
| | | | | A2301, A2402, | | | DPA10301- | | | |
| | | | | A2403, B3901, | | | DPB10402, | | | |
| | | | | B4506, C0702, | | | DQA10101- | | | |
| | | | | C1402, A2603, | | | DQB10501, | | | |
| | | | | A3201, B1503, | | | DQA10102- | | | |
| | | | | A2902, A8001, | | | DQB10502 | | | |
| | | | | B1502, B3801, | | | | | | |
| | | | | B1509, A2601, | | | | | | |
| | | | | A2603, A2902, | | | | | | |
| | | | | A3002, A3207, | | | | | | |
| | | | | A6601, A6823, | | | | | | |
| | | | | B1517, B3501, | | | | | | |
| | | | | B5701, C1203 | | | | | | |
| ORF1ab | FVKIGP | 89.7% | 42 | A0302, B1402, | 40.4% | 10 | DRB1_0101, | 0.66820875 | 0.95 | 2.92 |
| _6123 | ERTCCL | | | A3101, B0802, | | | DRB1_0402, | 7 | | |
| | CDRRA | | | B1402, C0501, | | | DRB1_0701, | | | |
| | TCFSTA | | | C0802, B1401, | | | DRB1_0801, | | | |
| | SDTYAC | | | B2705, B2720, | | | DRB1_1001, | | | |
| | WHHS | | | C0401, B5301, | | | DRB1_1201, | | | |
| | (SEQ ID | | | B5703, B5801, | | | DPA10103- | | | |
| | NO: 28) | | | C0303, C0501, | | | DPB10402, | | | |
| | | | | A2301, A2402, | | | DPA10301- | | | |
| | | | | A2403, B3901, | | | DPB10402, | | | |
| | | | | B4506, C0702, | | | DQA10101- | | | |
| | | | | C1402, A2603, | | | DQB10501, | | | |
| | | | | A3201, B1503, | | | DQA10102- | | | |
| | | | | A2902, A8001, | | | DQB10502 | | | |
| | | | | B1502, B3801, | | | | | | |
| | | | | B1509, A2601, | | | | | | |
| | | | | A2603, A2902, | | | | | | |
| | | | | A3002, A3207, | | | | | | |
| | | | | A6601, A6823, | | | | | | |
| | | | | B1517, B3501, | | | | | | |
| | | | | B5701, C1203 | | | | | | |
| ORF1ab | TRSTNS | 81.2% | 25 | C0602, C0701, | 66.9% | 9 | DRB1_0701, | 0.45854476 | 0.98 | 2.92 |
| _2183 | RIKASM | | | B1402, B2720, | | | DRB1_0901, | 1 | | |
| | PTTIAK | | | A3001, A0301, | | | DRB1_1301, | | | |
| | NTVKSV | | | A1101, A3001, | | | DRB1_1302, | | | |
| | GKFCLE | | | B5401, A1101, | | | DPA10103- | | | |
| | ASF | | | A6801, C1203, | | | DPB10201, | | | |
| | (SEQ ID | | | A2501, A2601, | | | DPA10103- | | | |
| | NO: 29) | | | A2602, A2603, | | | DPB10402, | | | |
| | | | | A6601, B5802, | | | DPA10103- | | | |
| | | | | B1503, A6601, | | | DPB10601, | | | |
| | | | | A8001, B1502, | | | DQA10102- | | | |
| | | | | B3501, A0212, | | | DQB10602, | | | |
| | | | | A6801 | | | DQA10501- | | | |
| | | | | | | | DQB10301 | | | |
| ORF1ab | IVVFDEI | 90.5% | 33 | B3501, B1801, | 42.8% | 3 | DPA10103- | 0.61824708 | 0.96 | 2.91 |
| _5694 | SMATN | | | B4402, B4403, | | | DPB10402, | 5 | | |
| | YDLSVV | | | B5701, C1502, | | | DQA10301- | | | |
| | NARLR | | | C1203, C1502, | | | DQB10302, | | | |
| | AKHYVY | | | A0301, A1101, | | | DQA10401- | | | |
| | IGDP | | | B0802, B2720, | | | DQB10402 | | | |
| | (SEQ ID | | | B4506, C0602, | | | | | | |
| | NO: 30) | | | C0701, A3001, | | | | | | |
| | | | | A3002, A3201, | | | | | | |
| | | | | A3207, A8001, | | | | | | |
| | | | | B0802, B0803, | | | | | | |
| | | | | B1502, B1503, | | | | | | |
| | | | | C0702, B7301, | | | | | | |
| | | | | A2603, A6823, | | | | | | |
| | | | | C1402, A2402, | | | | | | |
| | | | | A2403, C0802, | | | | | | |
| | | | | B8301 | | | | | | |
| M_15 | KLLEQ | 96.7% | 59 | A0201, A0206, | 29.2% | 2 | DPA10103- | 0.76418162 | 0.88 | 2.90 |
| | WNLVI | | | A0211, A0212, | | | DPB10201, | 4 | | |
| | GFLFLT | | | A0216, A0219, | | | DQA10301- | | | |
| | WICLLQ | | | A3201, A3207, | | | DQB10302 | | | |
| | FAYANR | | | A0217, B0803, | | | | | | |
| | NRFLY | | | A3201, B1503, | | | | | | |
| | (SEQ ID | | | B2720, B4013, | | | | | | |
| | NO: 31) | | | B4801, A2403, | | | | | | |
| | | | | C0401, A2902, | | | | | | |
| | | | | B3801, A0206, | | | | | | |
| | | | | B5301, A0207, | | | | | | |
| | | | | A0302, B1502, | | | | | | |
| | | | | B3503, B3901, | | | | | | |
| | | | | C1203, A2301, | | | | | | |
| | | | | B1517, B5701, | | | | | | |
| | | | | B5801, A0101, | | | | | | |
| | | | | A2902, A8001, | | | | | | |
| | | | | B3501, A6601, | | | | | | |
| | | | | B0802, B1402, | | | | | | |
| | | | | B4601, B5301, | | | | | | |
| | | | | C0303, C0602, | | | | | | |
| | | | | C0701, C0702, | | | | | | |
| | | | | C0802, C1402, | | | | | | |
| | | | | A3002, A6601, | | | | | | |
| | | | | A3001, B1401, | | | | | | |
| | | | | B7301, B2705, | | | | | | |
| | | | | A2402, A2403, | | | | | | |
| | | | | B0802, A2602, | | | | | | |
| | | | | A6823, B5703, | | | | | | |
| | | | | B5802 | | | | | | |
| M_38 | AYANR | 96.9% | 67 | A2403, C0602, | 15.8% | 5 | DRB1_0301, | 0.88453008 | 0.89 | 2.90 |
| | NRFLYII | | | C0701, C1402, | | | DRB1_0402, | 4 | | |
| | KLIFLW | | | A0101, A2902, | | | DRB1_0801, | | | |
| | LLWPV | | | A3002, A6601, | | | DPA10103- | | | |
| | TLACFV | | | A8001, B0802, | | | DPB10201, | | | |
| | LAAV | | | B3501, B5301, | | | DPA10103- | | | |
| | (SEQ ID | | | B5801, C0303, | | | DPB10601 | | | |
| | NO: 32) | | | C0702, C1203, | | | | | | |
| | | | | A3001, B1401, | | | | | | |
| | | | | B3901, B7301, | | | | | | |
| | | | | B2705, B2720, | | | | | | |
| | | | | B3801, A2301, | | | | | | |
| | | | | A2402, A2403, | | | | | | |
| | | | | A3207, B4013, | | | | | | |
| | | | | B0802, B0803, | | | | | | |
| | | | | B1502, B4601, | | | | | | |
| | | | | A2602, A6823, | | | | | | |
| | | | | B1517, B5701, | | | | | | |
| | | | | B5703, B5802, | | | | | | |
| | | | | B4801, A3201, | | | | | | |
| | | | | A0206, A0211, | | | | | | |
| | | | | A0216, A0219, | | | | | | |
| | | | | A6901, A0201, | | | | | | |
| | | | | A0207, B4801, | | | | | | |
| | | | | A0211, B1402, | | | | | | |
| | | | | A0205, B4201, | | | | | | |
| | | | | B5101, B8101, | | | | | | |
| | | | | A0202, A0203, | | | | | | |
| | | | | B1402, A3002, | | | | | | |
| | | | | B1503, A3301, | | | | | | |
| | | | | C0401, A0205, | | | | | | |
| | | | | A0206, A0212, | | | | | | |
| | | | | A0217, A6802, | | | | | | |
| | | | | B5301 | | | | | | |
| ORF1ab | TSRYW | 93.3% | 52 | A3207, B1517, | 43.4% | 8 | DRB1_0101, | 0.59132351 | 0.93 | 2.89 |
| _5304 | EPEFYE | | | B5703, A6823, | | | DRB1_0301, | 4 | | |
| | AMYTP | | | A8001, C0602, | | | DRB1_0401, | | | |
| | HTVLQ | | | C0701, C0702, | | | DRB1_0405, | | | |
| | AVGAC | | | A2403, A2602, | | | DPA10103- | | | |
| | VLCNSQ | | | A2603, B1801, | | | DPB10402, | | | |
| | T | | | B2720, B4001, | | | DPA10201- | | | |
| | (SEQ ID | | | B4002, B4801, | | | DPB10101, | | | |
| | NO: 33) | | | B3501, B3503, | | | DQA10101- | | | |
| | | | | B5301, C1402, | | | DQB10501, | | | |
| | | | | A6802, A6823, | | | DQA10501- | | | |
| | | | | A6901, B3901, | | | DQB10201 | | | |
| | | | | B5101, C1203, | | | | | | |
| | | | | C1502, A0211, | | | | | | |
| | | | | A0217, A3201, | | | | | | |
| | | | | B0803, B1402, | | | | | | |
| | | | | B1502, B1503, | | | | | | |
| | | | | B1509, B4013, | | | | | | |
| | | | | B4506, C0303, | | | | | | |
| | | | | C0401, C0401, | | | | | | |
| | | | | B0702, B4201, | | | | | | |
| | | | | B5401, B8101, | | | | | | |
| | | | | B8301, A0205, | | | | | | |
| | | | | A0211, A0212, | | | | | | |
| | | | | A0216, A0219, | | | | | | |
| | | | | B1501, A0302 | | | | | | |
| S_1205 | KYEQYI | 97.3% | 64 | A3207, A6601, | 29.2% | 2 | DPA10103- | 0.80918608 | 0.82 | 2.89 |
| | KWPW | | | A6823, B0803, | | | DPB10201, | 5 | | |
| | YIWLGF | | | B1801, B2720, | | | DQA10301- | | | |
| | IAGLIAI | | | B4402, B4403, | | | DQB10302 | | | |
| | VMVTI | | | B4506, B7301, | | | | | | |
| | MLCCM | | | A2603, A3207, | | | | | | |
| | (SEQ ID | | | B1502, B4013, | | | | | | |
| | NO: 34) | | | A0205, A2301, | | | | | | |
| | | | | A2402, A2403, | | | | | | |
| | | | | C0702, A3201, | | | | | | |
| | | | | B0802, B5703, | | | | | | |
| | | | | B5802, C1203, | | | | | | |
| | | | | B2720, B3701, | | | | | | |
| | | | | B4801, C0701, | | | | | | |
| | | | | A2402, B3501, | | | | | | |
| | | | | B3503, B4201, | | | | | | |
| | | | | B5101, B5301, | | | | | | |
| | | | | B8301, C0401, | | | | | | |
| | | | | B7301, A2403, | | | | | | |
| | | | | A0211, A0219, | | | | | | |
| | | | | A0201, A0202, | | | | | | |
| | | | | A0203, A0205, | | | | | | |
| | | | | A0206, A0207, | | | | | | |
| | | | | A0211, A0212, | | | | | | |
| | | | | A0216, A2601, | | | | | | |
| | | | | A6802, A6901, | | | | | | |
| | | | | B3501, B4601, | | | | | | |
| | | | | B5801, A0302, | | | | | | |
| | | | | A2501, B1517, | | | | | | |
| | | | | A0216, A0302, | | | | | | |
| | | | | A0301, A1101, | | | | | | |
| | | | | A3001, A6801 | | | | | | |
| ORF1ab | SMWAL | 97.1% | 65 | A0201, A0202, | 39.0% | 9 | DRB1_0401, | 0.57533916 | 0.95 | 2.89 |
| _3732 | IISVTSN | | | A0203, A0206, | | | DRB1_0403, | 4 | | |
| | YSGVVT | | | A0207, A0211, | | | DRB1_0404, | | | |
| | TVMFL | | | A0212, A0216, | | | DRB1_0802, | | | |
| | ARGIVF | | | A0217, A0219, | | | DRB1_1302, | | | |
| | MCVE | | | A3201, A6901, | | | DRB1_1501, | | | |
| | (SEQ ID | | | B1503, B2720, | | | DPA10103- | | | |
| | NO: 35) | | | B4013, B4506, | | | DPB10402, | | | |
| | | | | B4801, A2501, | | | DQA10103- | | | |
| | | | | A2601, A2902, | | | DQB10603, | | | |
| | | | | A3002, B1501, | | | DQA10501- | | | |
| | | | | B3501, A2602, | | | DQB10201 | | | |
| | | | | A6802, C1502, | | | | | | |
| | | | | A2403, B4601, | | | | | | |
| | | | | B5801, C0501, | | | | | | |
| | | | | C1502, A3301, | | | | | | |
| | | | | A3201, A2301, | | | | | | |
| | | | | A2402, A2403, | | | | | | |
| | | | | A3207, B0802, | | | | | | |
| | | | | B1502, C0401, | | | | | | |
| | | | | C0702, C1402, | | | | | | |
| | | | | A0205, A2602, | | | | | | |
| | | | | A6823, C0303, | | | | | | |
| | | | | C0802, C1203, | | | | | | |
| | | | | B2720, A2902, | | | | | | |
| | | | | A8001, A0302, | | | | | | |
| | | | | B0803, B3503, | | | | | | |
| | | | | B3901, B3501, | | | | | | |
| | | | | B5703, C0401, | | | | | | |
| | | | | A0211, A6601, | | | | | | |
| | | | | B3701, B4001, | | | | | | |
| | | | | B4002, B4402, | | | | | | |
| | | | | B4403 | | | | | | |
| ORF1ab | ETKFLT | 91.8% | 34 | A2501, A6802, | 51.7% | 7 | DPA10103- | 0.48241748 | 0.97 | 2.89 |
| _1245 | ENLLLYI | | | A6823, B1509, | | | DPB10201, | 4 | | |
| | DINGNL | | | B3801, B3901, | | | DPA10103- | | | |
| | HPDSAT | | | A2403, C0401, | | | DPB10401, | | | |
| | LVSDIDI | | | A0101, A2601, | | | DPA10201- | | | |
| | T | | | A2602, A2902, | | | DPB10101, | | | |
| | (SEQ ID | | | A8001, B1502, | | | DPA10301- | | | |
| | NO: 36) | | | B4601, C0602, | | | DPB10402, | | | |
| | | | | C0701, C0802, | | | DQA10102- | | | |
| | | | | C1203, B1801, | | | DQB10502, | | | |
| | | | | B4002, C1402, | | | DQA10301- | | | |
| | | | | C0501, A0212, | | | DQB10302, | | | |
| | | | | B1509, C0303, | | | DQA10401- | | | |
| | | | | B3501, A3201, | | | DQB10402 | | | |
| | | | | B3501, B5301, | | | | | | |
| | | | | A1101, A6801, | | | | | | |
| | | | | A2902, A3207 | | | | | | |
| ORF1ab | IAIILAS | 76.0% | 29 | A0202, A0203, | 79.5% | 15 | DRB1_0301, | 0.35375096 | 0.96 | 2.87 |
| _473 | FSASTSA | | | A0205, A2403, | | | DRB1_0401, | 4 | | |
| | FVETVK | | | B1502, C0401, | | | DRB1_0404, | | | |
| | GLDYKA | | | C1402, A0206, | | | DRB1_0701, | | | |
| | FKQIVE | | | A6802, A6901, | | | DRB1_0802, | | | |
| | S | | | C0303, C0501, | | | DRB1_0901, | | | |
| | (SEQ ID | | | C0802, C1203, | | | DRB1_1001, | | | |
| | NO: 37) | | | C1502, A6802, | | | DRB1_1501, | | | |
| | | | | A1101, A6801, | | | DRB1_1602, | | | |
| | | | | A8001, B4403, | | | DPA10201- | | | |
| | | | | A2501, A2603, | | | DPB11401, | | | |
| | | | | A6823, B1503, | | | DQA10102- | | | |
| | | | | A3001, A0302, | | | DQB10602, | | | |
| | | | | A2601, A2602, | | | DQA10103- | | | |
| | | | | B4001 | | | DQB10603, | | | |
| | | | | | | | DQA10301- | | | |
| | | | | | | | DQB10302, | | | |
| | | | | | | | DQA10401- | | | |
| | | | | | | | DQB10402, | | | |
| | | | | | | | DQA10501- | | | |
| | | | | | | | DQB10301 | | | |
| ORF1ab | FASEAA | 89.7% | 33 | B4601, B5101, | 62.7% | 9 | DRB1_0404, | 0.38791007 | 0.94 | 2.86 |
| _536 | RVVRSI | | | C0303, C0501, | | | DRB1_0701, | 2 | | |
| | FSRTLE | | | C0602, C0701, | | | DRB1_1201, | | | |
| | TAQNS | | | C0802, C1203, | | | DRB1_1301, | | | |
| | VRVLQK | | | C1502, B4501, | | | DRB1_1501, | | | |
| | AAIT | | | A6802, A6901, | | | DRB1_1602, | | | |
| | (SEQ ID | | | B1402, A1101, | | | DPA10103- | | | |
| | NO: 38) | | | A3101, A6601, | | | DPB10301, | | | |
| | | | | C0602, C0702, | | | DQA10102- | | | |
| | | | | A0205, A2601, | | | DQB10602, | | | |
| | | | | A6802, A3001, | | | DQA10501- | | | |
| | | | | A0211, A0212, | | | DQB10301 | | | |
| | | | | A0216, B0803, | | | | | | |
| | | | | B1501, B4801, | | | | | | |
| | | | | A2501, A2601, | | | | | | |
| | | | | A2602, A2902, | | | | | | |
| | | | | B1502 | | | | | | |
| ORF1ab | RTVYD | 96.7% | 60 | A2603, A6801, | 25.2% | 7 | DRB1_0101, | 0.67923005 | 0.96 | 2.85 |
| _3698 | DGARR | | | C0501, C0802, | | | DRB1_0301, | 1 | | |
| | VWTLM | | | B0801, B0802, | | | DRB1_0403, | | | |
| | NVLTLV | | | B1401, B1402, | | | DRB1_0801, | | | |
| | YKVYYG | | | A3001, B2720, | | | DRB1_0802, | | | |
| | NALDQ | | | B4506, C0602, | | | DRB1_1501, | | | |
| | A | | | A3201, B4013, | | | DPA10103- | | | |
| | (SEQ ID | | | B4801, C0401, | | | DPB10301 | | | |
| | NO: 39) | | | C1502, B1517, | | | | | | |
| | | | | C1402, A0201, | | | | | | |
| | | | | A0202, A0203, | | | | | | |
| | | | | A0206, A0207, | | | | | | |
| | | | | A0211, A0212, | | | | | | |
| | | | | A0216, A0217, | | | | | | |
| | | | | A0219, A0101, | | | | | | |
| | | | | A2902, A3002, | | | | | | |
| | | | | A3201, A8001, | | | | | | |
| | | | | B1501, B1502, | | | | | | |
| | | | | B1503, B3501, | | | | | | |
| | | | | B4601, B5801, | | | | | | |
| | | | | A0219, A6901, | | | | | | |
| | | | | A6823, B1517, | | | | | | |
| | | | | B5701, B1509, | | | | | | |
| | | | | B2720, B3701, | | | | | | |
| | | | | B3801, B3901, | | | | | | |
| | | | | B7301, C0303, | | | | | | |
| | | | | C0702, C1203, | | | | | | |
| | | | | C1402, A2403, | | | | | | |
| | | | | A6901, B5703, | | | | | | |
| | | | | C0501, B3901 | | | | | | |
| ORF1ab | MVLGS | 85.3% | 34 | A0206, A0203, | 63.0% | 13 | DRB1_0101, | 0.42181427 | 0.95 | 2.85 |
| _4241 | LAATVR | | | A0205, A0211, | | | DRB1_0401, | 7 | | |
| | LQAGN | | | A0212, A0216, | | | DRB1_0402, | | | |
| | ATEVPA | | | A0219, B1517, | | | DRB1_0403, | | | |
| | NSTVLS | | | B1402, B4001, | | | DRB1_0404, | | | |
| | FCAFA | | | B4506, C0401, | | | DRB1_0405, | | | |
| | (SEQ ID | | | A2602, B1503, | | | DRB1_0802, | | | |
| | NO: 40) | | | A2501, A2601, | | | DRB1_0901, | | | |
| | | | | A2602, A2603, | | | DRB1_1001, | | | |
| | | | | B1501, B1503, | | | DRB1_1101, | | | |
| | | | | B1517, C1402, | | | DRB1_1602, | | | |
| | | | | A6901, A0201, | | | DQA10102- | | | |
| | | | | A0202, B5401, | | | DQB10602, | | | |
| | | | | A6801, B1502, | | | DQA10501- | | | |
| | | | | B3501, B4601, | | | DQB10301 | | | |
| | | | | C0303, C0802, | | | | | | |
| | | | | C1203, A1101 | | | | | | |
| S_252 | GDSSSG | 95.7% | 53 | A2902, A3002, | 68.9% | 15 | DRB1_0101, | 0.47950716 | 0.71 | 2.84 |
| | WTAGA | | | B1502, C0401, | | | DRB1_0401, | 1 | | |
| | AAYYV | | | A0101, A2501, | | | DRB1_0402, | | | |
| | GYLQPR | | | A2601, A2602, | | | DRB1_0404, | | | |
| | TFLLKY | | | A2603, A2902, | | | DRB1_0405, | | | |
| | NENGT | | | A6601, A6801, | | | DRB1_0701, | | | |
| | (SEQ ID | | | A6823, A8001, | | | DRB1_0901, | | | |
| | NO: 41) | | | B1517, B3501, | | | DRB1_1001, | | | |
| | | | | B5703, B5801, | | | DRB1_1301, | | | |
| | | | | A0205, A0219, | | | DRB1_1501, | | | |
| | | | | A6802, A6901, | | | DRB1_1602, | | | |
| | | | | C1502, A6823, | | | DPA10103- | | | |
| | | | | C0303, A2403, | | | DPB10301, | | | |
| | | | | A2603, A3207, | | | DPA10301- | | | |
| | | | | A3301, C0702, | | | DPB10402, | | | |
| | | | | C1402, B1503, | | | DQA10102- | | | |
| | | | | A0201, A0202, | | | DQB10602, | | | |
| | | | | A0203, A0206, | | | DQA10501- | | | |
| | | | | A0211, A0212, | | | DQB10301 | | | |
| | | | | A0216, A0217, | | | | | | |
| | | | | B0801, B0802, | | | | | | |
| | | | | B0803, B1402, | | | | | | |
| | | | | B4013, C0602, | | | | | | |
| | | | | C0701, C0802, | | | | | | |
| | | | | C1203, B4506, | | | | | | |
| | | | | A8001, B8301, | | | | | | |
| | | | | B4501 | | | | | | |
| ORF1ab | NFTIKG | 90.3% | 43 | A0201, A0202, | 48.3% | 2 | DQA10102- | 0.52001215 | 0.93 | 2.83 |
| _3396 | SFLNGS | | | A0203, A0205, | | | DQB10602, | 4 | | |
| | CGSVGF | | | A0207, A0211, | | | DQA10501- | | | |
| | NIDYDC | | | A0212, A0216, | | | DQB10301 | | | |
| | VSFCY | | | A0217, A0219, | | | | | | |
| | MHHM | | | C0802, C0501, | | | | | | |
| | (SEQ ID | | | A0302, A2602, | | | | | | |
| | NO: 42) | | | A2603, A6823, | | | | | | |
| | | | | B1517, B4601, | | | | | | |
| | | | | B5701, B5801, | | | | | | |
| | | | | C0701, C1203, | | | | | | |
| | | | | C1502, A6823, | | | | | | |
| | | | | B0801, B0803, | | | | | | |
| | | | | B1509, B3503, | | | | | | |
| | | | | B4013, B4801, | | | | | | |
| | | | | C0303, C0602, | | | | | | |
| | | | | C0702, C1402, | | | | | | |
| | | | | A2403, A6601, | | | | | | |
| | | | | A8001, B1509, | | | | | | |
| | | | | B2720, B3801, | | | | | | |
| | | | | B3901, B4501, | | | | | | |
| | | | | B4506 | | | | | | |
| ORF1ab | DFIDTK | 93.5% | 40 | A0101, B0802, | 40.8% | 1 | DQA10501- | 0.50730622 | 0.98 | 2.83 |
| _220 | RGVYCC | | | C0501, C0802, | | | DQB10301 | 2 | | |
| | REHEHE | | | A0302, B4402, | | | | | | |
| | IAWYTE | | | B4403, B4801, | | | | | | |
| | RSEKSY | | | B1801, B4002, | | | | | | |
| | ELQ | | | A6601, B4506, | | | | | | |
| | (SEQ ID | | | C0401, A2501, | | | | | | |
| | NO: 43) | | | B1502, B1517, | | | | | | |
| | | | | B1401, B3901, | | | | | | |
| | | | | C0602, C0701, | | | | | | |
| | | | | C0702, B4501, | | | | | | |
| | | | | A3201, A3207, | | | | | | |
| | | | | A6601, A6823, | | | | | | |
| | | | | B1501, B1503, | | | | | | |
| | | | | B2720, B4013, | | | | | | |
| | | | | B4601, B5701, | | | | | | |
| | | | | B5801, B5802, | | | | | | |
| | | | | C1203, C1402, | | | | | | |
| | | | | C1502, B1801, | | | | | | |
| | | | | B4001, B4801 | | | | | | |
| ORF1ab | ALRQM | 86.3% | 27 | A3001, B0801, | 49.6% | 3 | DRB1_0401, | 0.48404068 | 0.97 | 2.81 |
| _4148 | SCAAGT | | | B2720, B4801, | | | DQA10102- | 5 | | |
| | TQTACT | | | B0803, C0303, | | | DQB10602, | | | |
| | DDNAL | | | A0101, A2902, | | | DQA10501- | | | |
| | AYYNTT | | | A8001, B1517, | | | DQB10301 | | | |
| | KGGRF | | | B3501, B4601, | | | | | | |
| | (SEQ ID | | | C0501, C0802, | | | | | | |
| | NO: 44) | | | C0501, A0301, | | | | | | |
| | | | | A1101, C1402, | | | | | | |
| | | | | A3301, C0401, | | | | | | |
| | | | | B0802, A6901, | | | | | | |
| | | | | A3207, B4013, | | | | | | |
| | | | | B2705, A0206, | | | | | | |
| | | | | A0217 | | | | | | |
| S_339 | GEVFN | 98.3% | 55 | B4001, B4402, | 36.5% | 8 | DRB1_0402, | 0.6903049 | 0.77 | 2.81 |
| | ATRFAS | | | B4403, A6601, | | | DRB1_0405, | | | |
| | VYAWN | | | A6802, A6901, | | | DRB1_1501, | | | |
| | RKRISN | | | A0205, B0801, | | | DPA10103- | | | |
| | CVADYS | | | B0802, B1402, | | | DPB10301, | | | |
| | VLYNS | | | C1203, C1502, | | | DPA10103- | | | |
| | (SEQ ID | | | B3501, A3001, | | | DPB10401, | | | |
| | NO: 45) | | | A3207, B2720, | | | DPA10103- | | | |
| | | | | B3801, B3901, | | | DPB10402, | | | |
| | | | | B7301, C0602, | | | DPA10201- | | | |
| | | | | C0701, C0702, | | | DPB10101, | | | |
| | | | | A3301, A6801, | | | DPA10301- | | | |
| | | | | A1101, A3001, | | | DPB10402 | | | |
| | | | | A0302, A2603, | | | | | | |
| | | | | A3101, A3301, | | | | | | |
| | | | | B4506, C0401, | | | | | | |
| | | | | A2402, C0602, | | | | | | |
| | | | | B2720, A3002, | | | | | | |
| | | | | A8001, A0I0l, | | | | | | |
| | | | | A2601, A2602, | | | | | | |
| | | | | A2902, B3501, | | | | | | |
| | | | | C0501, A2501, | | | | | | |
| | | | | A3201, A3207, | | | | | | |
| | | | | B1502, B1503, | | | | | | |
| | | | | B1517, C1402, | | | | | | |
| | | | | A2403, A6601, | | | | | | |
| | | | | B0803, A0301, | | | | | | |
| | | | | A1101 | | | | | | |
| ORF1ab | TEHSW | 86.2% | 49 | B3701, B3901, | 20.4% | 4 | DRB1_0101, | 0.79035823 | 0.95 | 2.81 |
| _6970 | NADLYK | | | B4001, C0401, | | | DRB1_0404, | 9 | | |
| | LMGHF | | | B3801, A1101, | | | DRB1_0701, | | | |
| | AWWT | | | A3001, A6801, | | | DRB1_0901 | | | |
| | AFVTN | | | A2403, A2602, | | | | | | |
| | VNASSS | | | A2501, A2601, | | | | | | |
| | EA | | | A2602, A2603, | | | | | | |
| | (SEQ ID | | | B1401, A3207, | | | | | | |
| | NO: 46) | | | B2720, B3801, | | | | | | |
| | | | | B4801, B7301, | | | | | | |
| | | | | C0702, A3201, | | | | | | |
| | | | | A3207, A6823, | | | | | | |
| | | | | B5703, B5801, | | | | | | |
| | | | | A0211, A0216, | | | | | | |
| | | | | A3001, B5401, | | | | | | |
| | | | | A3201, A6601, | | | | | | |
| | | | | B1402, B1502, | | | | | | |
| | | | | B1503, B1509, | | | | | | |
| | | | | B3701, B4013, | | | | | | |
| | | | | B4506, A0205, | | | | | | |
| | | | | A6802, A6901, | | | | | | |
| | | | | B3501, B1503, | | | | | | |
| | | | | C0303, B5801, | | | | | | |
| | | | | C1502, B3501, | | | | | | |
| | | | | C1402 | | | | | | |
| S_673 | SYQTQT | 94.5% | 47 | A2403, A2603, | 40.8% | 1 | DQA10501- | 0.59631992 | 0.85 | 2.80 |
| | NSPRRA | | | A6601, B1502, | | | DQB10301 | 6 | | |
| | RSVASQ | | | A3101, A3301, | | | | | | |
| | SIIAYT | | | B1402, B0702, | | | | | | |
| | MSLGA | | | B8301, A3001, | | | | | | |
| | ENSV | | | B2720, C0602, | | | | | | |
| | (SEQ ID | | | C0701, B1517, | | | | | | |
| | NO: 47) | | | B4801, B5801, | | | | | | |
| | | | | C1502, B1503, | | | | | | |
| | | | | B3501, B4601, | | | | | | |
| | | | | C1203, B3901, | | | | | | |
| | | | | B4013, A0201, | | | | | | |
| | | | | A0202, A0206, | | | | | | |
| | | | | A0217, A2501, | | | | | | |
| | | | | A2601, A2602, | | | | | | |
| | | | | A2603, A3201, | | | | | | |
| | | | | A3207, A6901, | | | | | | |
| | | | | B0801, B3901, | | | | | | |
| | | | | B4506, C1402, | | | | | | |
| | | | | A6823, C1502, | | | | | | |
| | | | | B3501, B4402, | | | | | | |
| | | | | B4403, B4501, | | | | | | |
| | | | | A3201, A6802, | | | | | | |
| | | | | C1203 | | | | | | |
| S_129 | KVCEFQ | 98.5% | 58 | C0401, A0201, | 39.0% | 9 | DRB1_0403, | 0.59611634 | 0.83 | 2.80 |
| | FCNDPF | | | A0202, A0206, | | | DRB1_0404, | 4 | | |
| | LGVYYH | | | A0211, A0212, | | | DRB1_0405, | | | |
| | KNNKS | | | A0216, A0217, | | | DRB1_1302, | | | |
| | WMESE | | | A6823, B1503, | | | DPA10103- | | | |
| | FRVYS | | | B1509, B2720, | | | DPB10201, | | | |
| | (SEQ ID | | | B3701, B3801, | | | DPA10103- | | | |
| | NO: 48) | | | B3901, B4001, | | | DPB10401, | | | |
| | | | | B4013, B4801, | | | DPA10103- | | | |
| | | | | C0303, C0401, | | | DPB10601, | | | |
| | | | | C0602, C0702, | | | DPA10301- | | | |
| | | | | C1203, A6901, | | | DPB10402, | | | |
| | | | | C0501, C1502, | | | DQA10102- | | | |
| | | | | A0101, A8001, | | | DQB10602 | | | |
| | | | | C0802, A6823, | | | | | | |
| | | | | A0301, A0302, | | | | | | |
| | | | | A1101, A3001, | | | | | | |
| | | | | A2301, A2402, | | | | | | |
| | | | | A2403, A3207, | | | | | | |
| | | | | C0701, C1402, | | | | | | |
| | | | | B1509, A3101, | | | | | | |
| | | | | A2602, B1502, | | | | | | |
| | | | | B1801, B4002, | | | | | | |
| | | | | B4403, B4501, | | | | | | |
| | | | | B4506, B7301, | | | | | | |
| | | | | B7301, A6601, | | | | | | |
| | | | | B0803, B1501, | | | | | | |
| | | | | B1517, B3501, | | | | | | |
| | | | | B4601, B5801 | | | | | | |
| ORF3a_ | ATIPIQ | 96.2% | 54 | A2501, A3207, | 32.7% | 6 | DRB1_0701, | 0.60162229 | 0.90 | 2.80 |
| 33 | ASLPFG | | | A6802, A6823, | | | DRB1_0901, | 6 | | |
| | WLIVG | | | B1509, B1517, | | | DPA10103- | | | |
| | VALLAV | | | B8301, C0303, | | | DPB10301, | | | |
| | FQSASK | | | C1402, C1502, | | | DPA10201- | | | |
| | IITL | | | A6601, B0702, | | | DPB11401, | | | |
| | (SEQ ID | | | B3501, B3503, | | | DQA10102- | | | |
| | NO: 49) | | | B4201, B5101, | | | DQB10602, | | | |
| | | | | B5301, B8101, | | | DQA10103- | | | |
| | | | | B5301, B5703, | | | DQB10603 | | | |
| | | | | B5801, C0303, | | | | | | |
| | | | | A3201, A6901, | | | | | | |
| | | | | B4013, B4506, | | | | | | |
| | | | | B5701, A0207, | | | | | | |
| | | | | B3501, B5401, | | | | | | |
| | | | | B5401, B4801, | | | | | | |
| | | | | A0202, A0203, | | | | | | |
| | | | | A0212, A0216, | | | | | | |
| | | | | A0219, B4801, | | | | | | |
| | | | | C0401, A0205, | | | | | | |
| | | | | B4601, A0201, | | | | | | |
| | | | | A0206, A0211, | | | | | | |
| | | | | A0301, A1101, | | | | | | |
| | | | | A6801, A1101, | | | | | | |
| | | | | A3001, B5701, | | | | | | |
| | | | | B0801, B0803, | | | | | | |
| | | | | B0802, C0702 | | | | | | |
| ORF7A_ | CPDGV | 95.0% | 49 | B3501, B5301, | 21.7% | 7 | DRB1_0101, | 0.64750475 | 0.98 | 2.79 |
| 67 | KHVYQL | | | B8301, B1401, | | | DRB1_0402, | 3 | | |
| | RARSVS | | | A3101, A6601, | | | DRB1_1201, | | | |
| | PKLFIR | | | B1402, C1402, | | | DPA10103- | | | |
| | QEEVQ | | | B0803, B2705, | | | DPB10401, | | | |
| | ELYSP | | | A3001, A3207, | | | DPA10103- | | | |
| | (SEQ ID | | | B2720, B4013, | | | DPB10601, | | | |
| | NO: 50) | | | B4801, C0401, | | | DPA10201- | | | |
| | | | | C1502, C0701, | | | DPB10101, | | | |
| | | | | B1517, B5801, | | | DPA10301- | | | |
| | | | | A1101, A3101, | | | DPB10402 | | | |
| | | | | A0201, A0202, | | | | | | |
| | | | | A0211, A0216, | | | | | | |
| | | | | C0602, C0701, | | | | | | |
| | | | | A2501, A2601, | | | | | | |
| | | | | A2602, A2603, | | | | | | |
| | | | | A6601, A6802, | | | | | | |
| | | | | A6823, A6901, | | | | | | |
| | | | | B0803, B1503, | | | | | | |
| | | | | B3701, B4001, | | | | | | |
| | | | | B4002, B4403, | | | | | | |
| | | | | A0211, A0217, | | | | | | |
| | | | | A0219, A2402, | | | | | | |
| | | | | A2403, B5401, | | | | | | |
| | | | | A3201 | | | | | | |
| ORF1ab | SEFSSLP | 78.5% | 33 | A3002, B1502, | 61.6% | 10 | DRB1_0401, | 0.42359844 | 0.97 | 2.79 |
| _3946 | SYAAFA | | | B1503, B1801, | | | DRB1_0901, | 6 | | |
| | TAQEAY | | | B4002, B4402, | | | DRB1_1001, | | | |
| | EQAVA | | | B4403, B4506, | | | DRB1_1501, | | | |
| | NGDSE | | | A6802, B5401, | | | DRB1_1602, | | | |
| | VVLK | | | A2403, A2602, | | | DQA10102- | | | |
| | (SEQ ID | | | A3207, B1517, | | | DQB10602, | | | |
| | NO: 51) | | | B3503, B4601, | | | DQA10103- | | | |
| | | | | B5801, B5802, | | | DQB10603, | | | |
| | | | | C0303, C0702, | | | DQA10301- | | | |
| | | | | C1402, A0205, | | | DQB10302, | | | |
| | | | | A0216, A0217, | | | DQA10401- | | | |
| | | | | B3501, C1402, | | | DQB10402, | | | |
| | | | | C0303, A2902, | | | DQA10501- | | | |
| | | | | B2720, B4501, | | | DQB10201 | | | |
| | | | | C1502, B0801, | | | | | | |
| | | | | B0803 | | | | | | |
| ORF1ab | NNLVV | 87.9% | 37 | A0201, A0202, | 54.5% | 5 | DRB1_0403, | 0.42614380 | 0.94 | 2.79 |
| _593 | MAYITG | | | A0203, A0205, | | | DRB1_0901, | 5 | | |
| | GVVQL | | | A0211, A0212, | | | DPA10103- | | | |
| | TSQWL | | | A0219, A6802, | | | DPB10301, | | | |
| | TNIFGT | | | A6901, A6901, | | | DQA10102- | | | |
| | VYEKLK | | | B5101, B5401, | | | DQB10602, | | | |
| | (SEQ ID | | | C0303, C1203, | | | DQA10501- | | | |
| | NO: 52) | | | A0216, A6601, | | | DQB10301 | | | |
| | | | | B1517, B5801, | | | | | | |
| | | | | A3201, A0203, | | | | | | |
| | | | | A0206, A0207, | | | | | | |
| | | | | A0216, A0101, | | | | | | |
| | | | | A8001, B1501, | | | | | | |
| | | | | B1503, A1101, | | | | | | |
| | | | | A6801, A2403, | | | | | | |
| | | | | C0401, A2603, | | | | | | |
| | | | | A6601, B1402, | | | | | | |
| | | | | C1402, C1502, | | | | | | |
| | | | | A0202 | | | | | | |
| ORF3a_ | HSYFTS | 96.3% | 51 | A0101, A2601, | 42.3% | 9 | DRB1_0101, | 0.47371136 | 0.92 | 2.78 |
| 204 | DYYQLY | | | A2902, A3002, | | | DRB1_0405, | | | |
| | STQLST | | | A6601, A6801, | | | DRB1_0901, | | | |
| | DTGVE | | | A6823, A8001, | | | DRB1_1602, | | | |
| | HVTFFI | | | B1503, B1517, | | | DPA10103- | | | |
| | YNKI | | | B3501, B5801, | | | DPB10201, | | | |
| | (SEQ ID | | | C1203, A2301, | | | DPA10103- | | | |
| | NO: 53) | | | A2402, A2403, | | | DPB10401, | | | |
| | | | | B0802, B4013, | | | DPA10201- | | | |
| | | | | C0303, C0401, | | | DPB10101, | | | |
| | | | | C0702, C1402, | | | DPA10301- | | | |
| | | | | A2501, A2602, | | | DPB10402, | | | |
| | | | | A2603, B0803, | | | DQA10101- | | | |
| | | | | B1502, B4506, | | | DQB10501 | | | |
| | | | | B4601, B5802, | | | | | | |
| | | | | C0602, C0701, | | | | | | |
| | | | | C0802, B1401, | | | | | | |
| | | | | B1509, B1503, | | | | | | |
| | | | | A0206, A6901, | | | | | | |
| | | | | C0501, A2602, | | | | | | |
| | | | | A6601, A8001, | | | | | | |
| | | | | B1801, B4403, | | | | | | |
| | | | | A0301, A0302, | | | | | | |
| | | | | A1101, A3001, | | | | | | |
| | | | | A3201, A6802, | | | | | | |
| | | | | A0211 | | | | | | |
| N_82 | DQIGYY | 95.1% | 45 | A3101, A3301, | 19.0% | 6 | DRB1_0103, | 0.61248734 | 0.89 | 2.65 |
| | RRATRR | | | A0302, A3101, | | | DRB1_0801, | 9 | | |
| | IRGGD | | | C0401, A2301, | | | DRB1_0901, | | | |
| | GKMKD | | | A2402, A2403, | | | DRB1_1101, | | | |
| | LSPRW | | | A6601, B0802, | | | DRB1_1301, | | | |
| | YFYYLG | | | B0803, C0602, | | | DPA10103- | | | |
| | (SEQ ID | | | C0701, C0702, | | | DPB10301 | | | |
| | NO: 56) | | | C1402, B7301, | | | | | | |
| | | | | B2705, B2720, | | | | | | |
| | | | | B0803, B5703, | | | | | | |
| | | | | B5801, B5802, | | | | | | |
| | | | | C0501, A3201, | | | | | | |
| | | | | A3207, A2601, | | | | | | |
| | | | | A2602, A2603, | | | | | | |
| | | | | A2902, A8001, | | | | | | |
| | | | | A0101, A3002, | | | | | | |
| | | | | A6823, B4506, | | | | | | |
| | | | | A3207, B0702, | | | | | | |
| | | | | B0801, B3503, | | | | | | |
| | | | | B4013, B4201, | | | | | | |
| | | | | B8101, B8301, | | | | | | |
| | | | | C0702, A2403, | | | | | | |
| | | | | B7301 | | | | | | |
| N_305 | AQFAPS | 87.5% | 40 | B1503, B4506, | 6.4% | 1 | DRB1_0901 | 0.46392035 | 0.91 | 2.31 |
| | ASAFFG | | | B4801, A2403, | | | | 3 | | |
| | MSRIG | | | A6601, B1502, | | | | | | |
| | MEVTP | | | C0401, C0702, | | | | | | |
| | SGTWL | | | C1402, A2601, | | | | | | |
| | TYTGAI | | | A6823, B0803, | | | | | | |
| | (SEQ ID | | | B1503, B3501, | | | | | | |
| | NO: 60) | | | B5703, C0303, | | | | | | |
| | | | | C0501, C1203, | | | | | | |
| | | | | A2603, A1101, | | | | | | |
| | | | | A3101, A6801, | | | | | | |
| | | | | A6823, B1517, | | | | | | |
| | | | | A0202, A0203, | | | | | | |
| | | | | A0211, A0212, | | | | | | |
| | | | | A0216, B4402, | | | | | | |
| | | | | B4403, A2602, | | | | | | |
| | | | | A2603, A8001, | | | | | | |
| | | | | B3503, B5301, | | | | | | |
| | | | | B8301, C0401, | | | | | | |
| | | | | A6901, C1502 | | | | | | |

TABLE 3

| | | | | | HLA | | | | | | | | | B and T |
|---|---|---|---|---|---|---|---|---|---|---|---|---|---|---|
| | | | HLA | | Class II | HLA | | | | | | B cell | | cell |
| | | | Class | | Popu- | Class | HLA | | | T Cell | | Confor- | | Total |
| Gene | | HLA Class I | I | | lation | II | Class | Dissim- | Conser- | Com- | B cell | ma- | B cell | Per- |
| Posi- | | Population | Alleles | | Presen- | Alleles | II | ilarity | vation | bined | Linear | tional | total | cen- |
| tion | Epitope | Presentation | Bound | HLA Class I binders | tation | Bound | binders | Score | Score | Score | 33mer | 33 mer | score | tile |
| S_53 | DLFLPFFS | A3207, A6601, B1402, | 34 | A0207, A6823, B3501, | 47.2% | 9 | DRB1_0401, | 0.60 | 0.81 | 2.73 | 0.86 | 0.44 | 1.30 | 93% |
| | NVTWFH | B1503, B1517, B3501, | | B3503, B5101, B5301, | | | DRB1_0701, | | | | | | | |
| | AIHVSGT | B4601, B5301, C0702, | | B5401, B8301, A2301, | | | DRB1_1602, | | | | | | | |
| | NGTKRFD | A2403, A2603, A2902, | | C0702, A3301, B5401, | | | DPA10103- | | | | | | | |
| | NPVLP | A3201, A8001, B0802, | | A3207, A6901, B3701, | | | DPB10201, | | | | | | | |
| | (SEQ ID | B0803, B1502, B4013, | | B3901, B4506, A0211, | | | DPA10103- | | | | | | | |
| | NO: 54) | A3201, A3207, A6802, | | A6802, C1502, B1509, | | | DPB10401, | | | | | | | |
| | | A6901, C1203, A2902, | | A2603, A3001, A3001, | | | DPA10103- | | | | | | | |
| | | A3002, A6802, A6823, | | B7301, C0602, C0701, | | | DPB10402, | | | | | | | |
| | | C0303, C0701, B1401, | | A2403, A3201, A3207, | | | DPA10103- | | | | | | | |
| | | C1402, A6823, B1509, | | C0401, C0501, A3002, | | | DPB10601, | | | | | | | |
| | | B3503, B3801, B3901, | | B1503 | | | DPA10201- | | | | | | | |
| | | B4201, B4801, B5101, | | | | | DPB10101, | | | | | | | |
| | | B5401, B8101, B8301, | | | | | DQA10102- | | | | | | | |
| | | C0401, A2602, A0302, | | | | | DQB10602 | | | | | | | |
| | | A1101, A3301, A6801, | | | | | | | | | | | | |
| | | B2705, B7301, C0602, | | | | | | | | | | | | |
| | | A3001, B7301, B5703, | | | | | | | | | | | | |
| | | A6901 | | | | | | | | | | | | |
| S_129 | KVCEFQF | 98.5% | 58 | C0401, A0201, A0202, | 39.0% | 9 | DRB1_0403, | 0.60 | 0.83 | 2.80 | 0.83 | 0.30 | 1.14 | 91% |
| | CNDPFLG | | | A0206, A0211, A0212, | | | DRB1_0404, | | | | | | | |
| | VYYHKNN | | | A0216, A0217, A6823, | | | DRB1_0405, | | | | | | | |
| | KSWMES | | | B1503, B1509, B2720, | | | DRB1_1302, | | | | | | | |
| | EFRVYS | | | B3701, B3801, B3901, | | | DPA10103- | | | | | | | |
| | (SEQ ID | | | B4001, B4013, B4801, | | | DPB10201, | | | | | | | |
| | NO: 48) | | | C0303, C0401, C0602, | | | DPA10103- | | | | | | | |
| | | | | C0702, C1203, A6901, | | | DPB10401, | | | | | | | |
| | | | | C0501, C1502, A0101, | | | DPA10103- | | | | | | | |
| | | | | A8001, C0802, A6823, | | | DPB10601, | | | | | | | |
| | | | | A0301, A0302, A1101, | | | DPA10301- | | | | | | | |
| | | | | A3001, A2301, A2402, | | | DPB10402, | | | | | | | |
| | | | | A2403, A3207, C0701, | | | DQA10102- | | | | | | | |
| | | | | C1402, B1509, A3101, | | | DQB10602 | | | | | | | |
| | | | | A2602, B1502, B1801, | | | | | | | | | | |
| | | | | B4002, B4403, B4501, | | | | | | | | | | |
| | | | | B4506, B7301, B7301, | | | | | | | | | | |
| | | | | A6601, B0803, B1501, | | | | | | | | | | |
| | | | | B1517, B3501, B4601, | | | | | | | | | | |
| | | | | B5801 | | | | | | | | | | |
| S_165 | NCTFEYVS | 84.3% | 36 | A2301, B1502, C0401, | 35.6% | 8 | DRB1_1001, | 0.39 | 0.87 | 2.46 | 0.79 | 0.40 | 1.19 | 82% |
| | QPFLMDL | | | C1402, A6823, B1509, | | | DRB1_1602, | | | | | | | |
| | EGKQGNF | | | B3503, B4001, B4002, | | | DPA10103- | | | | | | | |
| | KNLREFVF | | | B4013, B4506, B7301, | | | DPB10201, | | | | | | | |
| | KNI (SEQ | | | C0303, C0701, C0802, | | | DPA10103- | | | | | | | |
| | ID NO: | | | A2602, C0702, C1502, | | | DPB10301, | | | | | | | |
| | 58) | | | A3101, B0802, B1503, | | | DPA10103- | | | | | | | |
| | | | | A3001, A2501, A2601, | | | DPB10401, | | | | | | | |
| | | | | A2602, A2603, A2902, | | | DPA10201- | | | | | | | |
| | | | | A6601, A6823, A8001, | | | DPB10101, | | | | | | | |
| | | | | B1517, B3501, B4601, | | | DPA10301- | | | | | | | |
| | | | | C1203, A2403, B4013 | | | DPB10402, | | | | | | | |
| | | | | | | | DQA10501- | | | | | | | |
| | | | | | | | DQB10201 | | | | | | | |
| S_252 | GDSSSGW | 95.7% | 53 | A2902, A3002, B1502, | 68.9% | 15 | DRB1_0101, | 0.48 | 0.71 | 2.84 | 0.76 | 0.00 | 0.76 | 81% |
| | TAGAAAY | | | C0401, A0101, A2501, | | | DRB1_0401, | | | | | | | |
| | YVGYLQP | | | A2601, A2602, A2603, | | | DRB1_0402, | | | | | | | |
| | RTFLLKYN | | | A2902, A6601, A6801, | | | DRB1_0404, | | | | | | | |
| | ENGT | | | A6823, A8001, B1517, | | | DRB1_0405, | | | | | | | |
| | (SEQ ID | | | B3501, B5703, B5801, | | | DRB1_0701, | | | | | | | |
| | NO: 41) | | | A0205, A0219, A6802, | | | DRB1_0901, | | | | | | | |
| | | | | A6901, C1502, A6823, | | | DRB1_1001, | | | | | | | |
| | | | | C0303, A2403, A2603, | | | DRB1_1301, | | | | | | | |
| | | | | A3207, A3301, C0702, | | | DRB1_1501, | | | | | | | |
| | | | | C1402, B1503, A0201, | | | DRB1_1602, | | | | | | | |
| | | | | A0202, A0203, A0206, | | | DPA10103- | | | | | | | |
| | | | | A0211, A0212, A0216, | | | DPB10301, | | | | | | | |
| | | | | A0217, B0801, B0802, | | | DPA10301- | | | | | | | |
| | | | | B0803, B1402, B4013, | | | DPB10402, | | | | | | | |
| | | | | C0602, C0701, C0802, | | | DQA10102- | | | | | | | |
| | | | | C1203, B4506, A8001, | | | DQB10602, | | | | | | | |
| | | | | B8301, B4501 | | | DQA10501- | | | | | | | |
| | | | | | | | DQB10301 | | | | | | | |
| S_339 | GEVFNAT | 98.3% | 55 | B4001, B4402, B4403, | 36.5% | 8 | DRB1_0402, | 0.69 | 0.77 | 2.81 | 0.83 | 0.59 | 1.42 | 99% |
| | RFASVYA | | | A6601, A6802, A6901, | | | DRB1_0405, | | | | | | | |
| | WNRKRIS | | | A0205, B0801, B0802, | | | DRB1_1501, | | | | | | | |
| | NCVADYS | | | B1402, C1203, C1502, | | | DPA10103- | | | | | | | |
| | VLYNS | | | B3501, A3001, A3207, | | | DPB10301, | | | | | | | |
| | (SEQ ID | | | B2720, B3801, B3901, | | | DPA10103- | | | | | | | |
| | NO: 45) | | | B7301, C0602, C0701, | | | DPB10401, | | | | | | | |
| | | | | C0702, A3301, A6801, | | | DPA10103- | | | | | | | |
| | | | | A1101, A3001, A0302, | | | DPB10402, | | | | | | | |
| | | | | A2603, A3101, A3301, | | | DPA10201- | | | | | | | |
| | | | | B4506, C0401, A2402, | | | DPB10101, | | | | | | | |
| | | | | C0602, B2720, A3002, | | | DPA10301- | | | | | | | |
| | | | | A8001, A0101, A2601, | | | DPB10402 | | | | | | | |
| | | | | A2602, A2902, B3501, | | | | | | | | | | |
| | | | | C0501, A2501, A3201, | | | | | | | | | | |
| | | | | A3207, B1502, B1503, | | | | | | | | | | |
| | | | | B1517, C1402, A2403, | | | | | | | | | | |
| | | | | A6601, B0803, A0301, | | | | | | | | | | |
| | | | | A1101 | | | | | | | | | | |
| S_394 | NVYADSF | 82.9% | 26 | A3207, A6802, A6901, | 20.8% | 2 | DPA10103- | 0.41 | 0.79 | 2.23 | 0.83 | 0.74 | 1.57 | 87% |
| | VIRGDEV | | | B4013, C1502, A3101, | | | DPB10402, | | | | | | | |
| | RQIAPGQ | | | A3301, C0501, C0802, | | | DQA10501- | | | | | | | |
| | TGKIADY | | | A6801, C0602, C0701, | | | DQB10201 | | | | | | | |
| | NYKLP | | | A2603, B1503, B4801, | | | | | | | | | | |
| | (SEQ ID | | | A0201, A0202, A0212, | | | | | | | | | | |
| | NO: 63) | | | A0217, A3201, B4201, | | | | | | | | | | |
| | | | | A0207, A6901, B8101, | | | | | | | | | | |
| | | | | B8301, C0501 | | | | | | | | | | |
| S_445 | VGGNYNY | 88.0% | 34 | A2902, A3002, A8001, | 10.6% | 4 | DRB1_0403, | 0.42 | 0.77 | 2.18 | 0.90 | 0.79 | 1.69 | 89% |
| | LYRLFRKS | | | B1401, B4801, A2301, | | | DRB1_0801, | | | | | | | |
| | NLKPFER | | | A2402, A2403, C0702, | | | DRB1_1101, | | | | | | | |
| | DISTEIYQ | | | A0302, A3301, A6801, | | | DPA10201- | | | | | | | |
| | AGS (SEQ | | | A6823, A3301, B1402, | | | DPB10501 | | | | | | | |
| | ID NO: | | | B2705, B2720, C0602, | | | | | | | | | | |
| | 65) | | | C0701, C1402, A0301, | | | | | | | | | | |
| | | | | A1101, A3001, A3101, | | | | | | | | | | |
| | | | | B0802, B0803, B1502, | | | | | | | | | | |
| | | | | B1503, C1203, A3101, | | | | | | | | | | |
| | | | | B8301, B4002, B4506, | | | | | | | | | | |
| | | | | C1402 | | | | | | | | | | |
| S_462 | KPFERDIS | 74.8% | 27 | B8301, B4002, B4506, | 18.7% | 5 | DRB1_0701, | 0.51 | 0.77 | 2.21 | 0.89 | 0.40 | 1.29 | 75% |
| | TEIYQAGS | | | C1203, C1402, B4201, | | | DRB1_0801, | | | | | | | |
| | TPCNGVE | | | B8301, A3207, A6601, | | | DRB1_1101, | | | | | | | |
| | GFNCYFPL | | | A6802, B2720, B4013, | | | DRB1_1602, | | | | | | | |
| | QS (SEQ | | | B4801, C0401, A6823, | | | DPA10201- | | | | | | | |
| | ID NO: | | | B1402, B1502, A2403, | | | DPB10501 | | | | | | | |
| | 64) | | | A2402, A2403, B0802, | | | | | | | | | | |
| | | | | C0401, C0702, C1402, | | | | | | | | | | |
| | | | | B3503, A0206, B1503 | | | | | | | | | | |
| S_490 | FPLQSYGF | 89.6% | 36 | B3503, A0206, B1503, | 13.4% | 1 | DQA10101- | 0.51 | 0.76 | 2.30 | 0.77 | 0.80 | 1.57 | 89% |
| | QPTNGVG | | | B2720, A6601, A6901, | | | DQB10501 | | | | | | | |
| | YQPYRVV | | | C1203, B1502, B4506, | | | | | | | | | | |
| | VLSFELLH | | | C1203, C0702, C1402, | | | | | | | | | | |
| | APA (SEQ | | | B0803, B1402, B3701, | | | | | | | | | | |
| | ID NO: | | | B3901, B4013, B4801, | | | | | | | | | | |
| | 61) | | | C0401, C0602, C0701, | | | | | | | | | | |
| | | | | C0702, A2301, A2402, | | | | | | | | | | |
| | | | | A2403, A3201, B1517, | | | | | | | | | | |
| | | | | C1502, A8001, B1801, | | | | | | | | | | |
| | | | | B4002, A0205, A0212, | | | | | | | | | | |
| | | | | A0216, A0219, B8301 | | | | | | | | | | |
| S_673 | SYQTQTN | 94.5% | 47 | A2403, A2603, A6601, | 40.8% | 1 | DQA10501- | 0.60 | 0.85 | 2.80 | 0.99 | 0.46 | 1.45 | 100% |
| | SPRRARS | | | B1502, A3101, A3301, | | | DQB10301 | | | | | | | |
| | VASQSIIA | | | B1402, B0702, B8301, | | | | | | | | | | |
| | YTMSLGA | | | A3001, B2720, C0602, | | | | | | | | | | |
| | ENSV | | | C0701, B1517, B4801, | | | | | | | | | | |
| | (SEQ ID | | | B5801, C1502, B1503, | | | | | | | | | | |
| | NO: 47) | | | B3501, B4601, C1203, | | | | | | | | | | |
| | | | | B3901, B4013, A0201, | | | | | | | | | | |
| | | | | A0202, A0206, A0217, | | | | | | | | | | |
| | | | | A2501, A2601, A2602, | | | | | | | | | | |
| | | | | A2603, A3201, A3207, | | | | | | | | | | |
| | | | | A6901, B0801, B3901, | | | | | | | | | | |
| | | | | B4506, C1402, A6823, | | | | | | | | | | |
| | | | | C1502, B3501, B4402, | | | | | | | | | | |
| | | | | B4403, B4501, A3201, | | | | | | | | | | |
| | | | | A6802, C1203 | | | | | | | | | | |
| S_762 | QLNRALT | 81.1% | 35 | A0203, A0219, B1402, | 52.6% | 7 | DRB1_0401, | 0.44 | 0.79 | 2.57 | 0.80 | 0.43 | 1.23 | 87% |
| | GIAVEQD | | | B3901, B7301, C0802, | | | DRB1_0405, | | | | | | | |
| | KNTQEVF | | | A0206, A0212, A6802, | | | DRB1_1501, | | | | | | | |
| | AQVKQIY | | | A6901, C1502, A2501, | | | DPA10103- | | | | | | | |
| | KTPPI | | | B0802, B0803, A3001, | | | DPB10301, | | | | | | | |
| | (SEQ ID | | | A3201, A3207, A6601, | | | DQA10301- | | | | | | | |
| | NO: 570 | | | A6823, B1503, B2720, | | | DQB10302, | | | | | | | |
| | | | | B4013, B4506, B4801, | | | DQA10401- | | | | | | | |
| | | | | A0301, A6601, B1502, | | | DQB10402, | | | | | | | |
| | | | | C0303, C0701, C0702, | | | DQA10501- | | | | | | | |
| | | | | C1203, A2602, A2603, | | | DQB10201 | | | | | | | |
| | | | | B3503, C0501 | | | | | | | | | | |
| S_806 | LPDPSKPS | 79.4% | 23 | B8301, A3207, B1503, | 26.5% | 4 | DRB1_0402, | 0.41 | 0.80 | 2.27 | 0.83 | 0.47 | 1.31 | 80% |
| | KRSFIEDL | | | B1517, B4013, B5701, | | | DRB1_0405, | | | | | | | |
| | LFNKVTLA | | | B5801, C1502, A1101, | | | DPA10103- | | | | | | | |
| | DAGFIKQ | | | A0201, A0202, A0203, | | | DPB10401, | | | | | | | |
| | YG (SEQ | | | A0216, B1517, C0501, | | | DPA10103- | | | | | | | |
| | ID NO: | | | B4403, B0801, B0802, | | | DPB10402 | | | | | | | |
| | 62) | | | B0803, C0303, C0802, | | | | | | | | | | |
| | | | | C1203, A2403 | | | | | | | | | | |
| S_882 | ITSGWTF | 96.0% | 60 | A6802, A6901, A2602, | 76.9% | 11 | DRB1_0101, | 0.51 | 0.80 | 3.03 | 0.54 | 0.57 | 1.11 | 97% |
| | GAGAALQ | | | A2603, A6802, A6823, | | | DRB1_0402, | | | | | | | |
| | IPFAMQ | | | B1502, B1509, B1517, | | | DRB1_0404, | | | | | | | |
| | MAYRFN | | | B3801, B3901, B4013, | | | DRB1_0701, | | | | | | | |
| | GIGVTQN | | | B4801, C0303, C0401, | | | DRB1_0901, | | | | | | | |
| | (SEQ ID | | | C1203, C1402, C1502, | | | DRB1_1501, | | | | | | | |
| | NO: 17) | | | C0401, A3207, B3501, | | | DQA10102- | | | | | | | |
| | | | | B4601, A0206, B1501, | | | DQB10602, | | | | | | | |
| | | | | B1503, B2720, B4506, | | | DQA10103- | | | | | | | |
| | | | | A2902, A8001, B1801, | | | DQB10603, | | | | | | | |
| | | | | B3503, B5101, B5301, | | | DQA10301- | | | | | | | |
| | | | | B8101, B8301, A3301, | | | DQB10302, | | | | | | | |
| | | | | A2301, A2402, A2403, | | | DQA10401- | | | | | | | |
| | | | | A2902, A3201, A3207, | | | DQB10402, | | | | | | | |
| | | | | A6601, B0801, B0802, | | | DQA10501- | | | | | | | |
| | | | | B0803, B5703, B5801, | | | DQB10301 | | | | | | | |
| | | | | B5802, C0501, C0602, | | | | | | | | | | |
| | | | | C0702, B4013, A0203, | | | | | | | | | | |
| | | | | A0212, B1402, A3001, | | | | | | | | | | |
| | | | | B2720, B7301, C0701 | | | | | | | | | | |
| S_1030 | SECVLGQ | 88.8% | 41 | A8001, C0501, C0303, | 51.2% | 4 | DRB1_0404, | 0.46 | 0.84 | 2.70 | 0.69 | 0.32 | 1.01 | 84% |
| | SKRVDFC | | | C0802, B1402, B1503, | | | DPA10103- | | | | | | | |
| | GKGYHL | | | A0201, A0202, A0203, | | | DPB10301, | | | | | | | |
| | MSFPQSA | | | A3207, A6601, A6823, | | | DQA10102- | | | | | | | |
| | PHGVVF | | | B1517, B3501, B4601, | | | DQB10602, | | | | | | | |
| | (SEQ ID | | | C0303, A6901, B0802, | | | DQA10501- | | | | | | | |
| | NO: 55) | | | B3901, B4201, B5101, | | | DQB10301 | | | | | | | |
| | | | | B5301, B5401, B8101, | | | | | | | | | | |
| | | | | B1501, C1203, A6823, | | | | | | | | | | |
| | | | | B8301, A2902, A8001, | | | | | | | | | | |
| | | | | B4506, A0205, A0206, | | | | | | | | | | |
| | | | | A0219, C0602, C0701, | | | | | | | | | | |
| | | | | A0203, A0211, A0212, | | | | | | | | | | |
| | | | | A0216, B0803 | | | | | | | | | | |
| S_1081 | ICHDGKA | 90.9% | 41 | C0501, A6601, B1402, | 8.0% | 2 | DRB1_0405, | 0.62 | 0.81 | 2.42 | 0.71 | 0.87 | 1.58 | 92% |
| | HFPREGV | | | B1509, B4013, C0702, | | | DQA10103- | | | | | | | |
| | FVSNGTH | | | A0207, B3501, B5401, | | | DQB10603 | | | | | | | |
| | WFVTQR | | | A2301, B5801, A2403, | | | | | | | | | | |
| | NFYEPQ | | | A2501, A2601, A2602, | | | | | | | | | | |
| | (SEQ ID | | | A6601, A6823, B1502, | | | | | | | | | | |
| | NO: 59) | | | B3501, B4601, C0501, | | | | | | | | | | |
| | | | | C0701, C1203, C1402, | | | | | | | | | | |
| | | | | A0211, A0216, A0219, | | | | | | | | | | |
| | | | | C1502, A0302, A3101, | | | | | | | | | | |
| | | | | A6801, A2403, A2902, | | | | | | | | | | |
| | | | | C0401, B0802, B2720, | | | | | | | | | | |
| | | | | B7301, A3207, B3701, | | | | | | | | | | |
| | | | | C0702, A2501 | | | | | | | | | | |
| M_15 | KLLEQWN | 96.7% | 59 | A0201, A0206, A0211, | 29.2% | 2 | DPA10103- | 0.76 | 0.88 | 2.90 | 0.68 | 0.00 | 0.68 | 80% |
| | LVIGFLFLT | | | A0212, A0216, A0219, | | | DPB10201, | | | | | | | |
| | WICLLQF | | | A3201, A3207, A0217, | | | DQA10301- | | | | | | | |
| | AYANRNR | | | B0803, A3201, B1503, | | | DQB10302 | | | | | | | |
| | FLY (SEQ | | | B2720, B4013, B4801, | | | | | | | | | | |
| | ID NO: | | | A2403, C0401, A2902, | | | | | | | | | | |
| | 31) | | | B3801, A0206, B5301, | | | | | | | | | | |
| | | | | A0207, A0302, B1502, | | | | | | | | | | |
| | | | | B3503, B3901, C1203, | | | | | | | | | | |
| | | | | A2301, B1517, B5701, | | | | | | | | | | |
| | | | | B5801, A0101, A2902, | | | | | | | | | | |
| | | | | A8001, B3501, A6601, | | | | | | | | | | |
| | | | | B0802, B1402, B4601, | | | | | | | | | | |
| | | | | B5301, C0303, C0602, | | | | | | | | | | |
| | | | | C0701, C0702, C0802, | | | | | | | | | | |
| | | | | C1402, A3002, A6601, | | | | | | | | | | |
| | | | | A3001, B1401, B7301, | | | | | | | | | | |
| | | | | B2705, A2402, A2403, | | | | | | | | | | |
| | | | | B0802, A2602, A6823, | | | | | | | | | | |
| | | | | B5703, B5802 | | | | | | | | | | |
| M_87 | LVGLMW | 97.9% | 66 | A0101, A2501, A2902, | 70.4% | 12 | DRB1_0402, | 0.76 | 0.93 | 3.37 | 0.63 | 0.00 | 0.63 | 92% |
| | LSYFIASFR | | | A3201, A6601, A8001, | | | DRB1_0403, | | | | | | | |
| | LFARTRS | | | B5703, A0201, A0202, | | | DRB1_1501, | | | | | | | |
| | MWSFNP | | | A0203, A0206, A0211, | | | DRB1_1602, | | | | | | | |
| | ETNIL | | | A0212, A0216, A0217, | | | DPA10103- | | | | | | | |
| | (SEQ ID | | | A0219, A3207, A6823, | | | DPB10201, | | | | | | | |
| | NO: 5) | | | B4013, B4506, B5401, | | | DPA10103- | | | | | | | |
| | | | | B7301, A0205, A2602, | | | DPB10401, | | | | | | | |
| | | | | A2603, B0802, B0803, | | | DPA10103- | | | | | | | |
| | | | | B1501, B1502, B1503, | | | DPB10601, | | | | | | | |
| | | | | A0301, A0302, A1101, | | | DPA10201- | | | | | | | |
| | | | | A3101, A3301, A6801, | | | DPB10101, | | | | | | | |
| | | | | A2301, A2402, A2403, | | | DQA10101- | | | | | | | |
| | | | | C0702, C1402, C0401, | | | DQB10501, | | | | | | | |
| | | | | A0202, A6802, A3101, | | | DQA10102- | | | | | | | |
| | | | | B1402, A0302, B7301, | | | DQB10502, | | | | | | | |
| | | | | A3201, B0801, B2720, | | | DQA10102- | | | | | | | |
| | | | | C0602, C0701, C1203, | | | DQB10602, | | | | | | | |
| | | | | A2403, B5703, B0802, | | | DQA10501- | | | | | | | |
| | | | | A2602, B1401, B2705, | | | DQB10301 | | | | | | | |
| | | | | A3001, A3207, C1502, | | | | | | | | | | |
| | | | | A0217, C0802, B3901 | | | | | | | | | | |
| S_603 | NTSNQVA | | | | | | | | | | | | | |
| | VLYQGVN | | | | | | | | | | | | | |
| | CTEVPVAI | | | | | | | | | | | | | |
| | HADQLTP | | | | | | | | | | | | | |
| | TWRV | | | | | | | | | | | | | |
| | (SEQ ID | | | | | | | | | | | | | |
| | NO: 104) | | | | | | | | | | | | | |

TABLE 4
Prioritized List of Sixty-five 33-mer Peptide Sequences Enriched for Population-Scale Immunity.

| | | | | HLA | | HLA | | | | |
|---|---|---|---|---|---|---|---|---|---|---|
| | | SEQ | HLA | Class | HLA | Class | Dissim- | Conser- | T | B |
| Gene | | ID | Class | I | Class | II | ilarity | vation | Cell | cell |
| Position | Epitope | NO | I | Bound | II | Bound | Score | Score | Score | Score |
| ORF1ab_3619 | IAMSAFAMMFVKHK | 1 | 98.6% | 74 | 82.1% | 24 | 0.82 | 0.96 | 3.59 | NA |
| | HAFLCLFLLPSLAT | | | | | | | | | |
| | VAYFN | | | | | | | | | |
| ORF1ab_2331 | YILFTRFFYVLGLAAI | 2 | 97.6% | 70 | 69.4% | 16 | 0.83 | 0.97 | 3.47 | NA |
| | MQLFFSYFAVHFISNS | | | | | | | | | |
| | W | | | | | | | | | |
| ORF1ab_2354 | FAVHFISNSWLMWLI | 3 | 99.2% | 80 | 55.3% | 14 | 0.95 | 0.97 | 3.46 | NA |
| | INLVQMAPISAMVRM | | | | | | | | | |
| | YIF | | | | | | | | | |
| ORF1ab_3057 | VTCLAYYFMRFRRAF | 4 | 98.4% | 82 | 64.1% | 11 | 0.87 | 0.97 | 3.46 | NA |
| | GEYSHVVAFNTLLFL | | | | | | | | | |
| | MSF | | | | | | | | | |
| M_87 | LVGLMWLSYFIASFR | 5 | 97.9% | 66 | 70.4% | 12 | 0.76 | 0.93 | 3.37 | 0.63 |
| | LFARTRSMWSFNPET | | | | | | | | | |
| | NIL | | | | | | | | | |
| ORF1ab_3123 | LAHIQWMVMFTPLVP | 6 | 97.4% | 75 | 34.3% | 9 | 0.99 | 0.98 | 3.29 | NA |
| | FWITIAYIICISTKH | | | | | | | | | |
| | FYW | | | | | | | | | |
| ORF1ab_4978 | TVVIGTSKFYGGWHN | 7 | 96.6% | 53 | 62.5% | 10 | 0.72 | 0.96 | 3.26 | NA |
| | MLKTVYSDVENPHL | | | | | | | | | |
| | MGWD | | | | | | | | | |
| ORF1ab_3804 | FRLTLGVYDYLVSTQ | 8 | 96.6% | 47 | 54.5% | 10 | 0.71 | 0.96 | 3.18 | NA |
| | EFRYMNSQGLLPPKN | | | | | | | | | |
| | SID | | | | | | | | | |
| ORF1ab_3783 | YCFLGYFCTCYFGLF | 9 | 96.6% | 52 | 43.9% | 9 | 0.82 | 0.95 | 3.18 | NA |
| | CLLNRYFRLTLGVYD | | | | | | | | | |
| | YLV | | | | | | | | | |
| ORF1ab_5147 | MMILSDDAVVCFNST | 10 | 96.4% | 47 | 60.8% | 21 | 0.65 | 0.95 | 3.17 | NA |
| | YASQGLVASIKNFKS | | | | | | | | | |
| | VLY | | | | | | | | | |
| ORF3a_119 | NFVRIIMRLWLCWKC | 11 | 98.0% | 63 | 46.5% | 15 | 0.8 | 0.91 | 3.15 | NA |
| | RSKNPLLYDANYFLC | | | | | | | | | |
| | WHT | | | | | | | | | |
| ORF1ab_6417 | RLYLDAYNMMISAGF | 12 | 99.2% | 73 | 48.4% | 12 | 0.7 | 0.95 | 3.13 | NA |
| | SLWVYKQFDTYNLW | | | | | | | | | |
| | NTFT | | | | | | | | | |
| ORF1ab_1799 | QQESPFVMMSAPPAQ | 13 | 94.8% | 51 | 54.7% | 9 | 0.64 | 0.96 | 3.09 | NA |
| | YELKHGTFTCASEYT | | | | | | | | | |
| | GNY | | | | | | | | | |
| ORF1ab_3196 | DVLLPLTQYNRYLAL | 14 | 96.6% | 60 | 36.5% | 10 | 0.78 | 0.98 | 3.09 | NA |
| | YNKYKYFSGAMDTT | | | | | | | | | |
| | SYRE | | | | | | | | | |
| ORF1ab_6658 | RSQMEIDFLELAMDE | 15 | 90.5% | 32 | 59.4% | 8 | 0.61 | 0.95 | 3.07 | NA |
| | FIERYKLEGYAFEHI | | | | | | | | | |
| | VYG | | | | | | | | | |
| ORF1ab_1624 | DDTLRVEAFEYYHTT | 16 | 96.9% | 57 | 38.1% | 3 | 0.79 | 0.90 | 3.04 | NA |
| | DPSFLGRYMSALNHT | | | | | | | | | |
| | KKW | | | | | | | | | |
| S_882 | ITSGWTFGAGAALQI | 17 | 96.0% | 60 | 76.9% | 11 | 0.51 | 0.80 | 3.03 | 1.11 |
| | PFAMQMAYRFNGIGV | | | | | | | | | |
| | TQN | | | | | | | | | |
| ORF1ab_2562 | SQLMCQPILLLDQAL | 18 | 95.2% | 47 | 54.0% | 3 | 0.55 | 0.96 | 3.00 | NA |
| | VSDVGDSAEVAVKM | | | | | | | | | |
| | FDAY | | | | | | | | | |
| ORF1ab_5027 | SLVLARKHTTCCSLS | 19 | 96.6% | 55 | 41.6% | 11 | 0.66 | 0.95 | 2.99 | NA |
| | HRFYRLANECAQVLS | | | | | | | | | |
| | EMV | | | | | | | | | |
| ORF1ab_5028 | LVLARKHTTCCSLSH | 20 | 96.6% | 55 | 41.6% | 11 | 0.66 | 0.95 | 2.99 | NA |
| | RFYRLANECAQVLSE | | | | | | | | | |
| | MVM | | | | | | | | | |
| ORF1ab_5029 | VLARKHTTCCSLSHR | 21 | 96.6% | 55 | 41.6% | 11 | 0.66 | 0.95 | 2.99 | NA |
| | FYRLANECAQVLSEM | | | | | | | | | |
| | VMC | | | | | | | | | |
| ORF1ab_5974 | MTYRRLISMMGFKM | 22 | 93.1% | 54 | 37.1% | 11 | 0.73 | 0.96 | 2.99 | NA |
| | NYQVNGYPNMFITRE | | | | | | | | | |
| | EAIR | | | | | | | | | |
| ORF1ab_4100 | QVVDADSKIVQLSEIS | 23 | 90.8% | 29 | 59.4% | 8 | 0.51 | 0.97 | 2.98 | NA |
| | MDNSPNLAWPLIVTA | | | | | | | | | |
| | LR | | | | | | | | | |
| ORF1ab_4622 | GVPVVDSYYSLLMPI | 24 | 95.0% | 38 | 61.5% | 6 | 0.45 | 0.95 | 2.97 | NA |
| | LTLTRALTAESHVDT | | | | | | | | | |
| | DLT | | | | | | | | | |
| ORF1ab_5258 | AYPLTKHPNQEYADV | 25 | 96.8% | 55 | 44.9% | 8 | 0.63 | 0.93 | 2.97 | NA |
| | FHLYLQYIRKLHDEL | | | | | | | | | |
| | TGH | | | | | | | | | |
| ORF1ab_2251 | GVLMSNLGMPSYCT | 26 | 91.0% | 41 | 61.6% | 10 | 0.48 | 0.95 | 2.95 | NA |
| | GYREGYLNSTNVTI | | | | | | | | | |
| | ATYCT | | | | | | | | | |
| ORF1ab_6122 | YFVKIGPERTCCLCD | 27 | 89.7% | 42 | 40.4% | 10 | 0.67 | 0.95 | 2.92 | NA |
| | RRATCFSTASDTYAC | | | | | | | | | |
| | WHH | | | | | | | | | |
| ORF1ab_6123 | FVKIGPERTCCLCDR | 28 | 89.7% | 42 | 40.4% | 10 | 0.67 | 0.95 | 2.92 | NA |
| | RATCFSTASDTYACW | | | | | | | | | |
| | HHS | | | | | | | | | |
| ORF1ab_2183 | TRSTNSRIKASMPTTI | 29 | 81.2% | 25 | 66.9% | 9 | 0.46 | 0.98 | 2.92 | NA |
| | AKNTVKSVGKFCLEA | | | | | | | | | |
| | SF | | | | | | | | | |
| ORF1ab_5694 | IVVFDEISMATNYDLS | 30 | 90.5% | 33 | 42.8% | 3 | 0.62 | 0.96 | 2.91 | NA |
| | VVNARLRAKHYVYIG | | | | | | | | | |
| | DP | | | | | | | | | |
| M_15 | KLLEQWNLVIGFLFL | 31 | 96.7% | 59 | 29.2% | 2 | 0.76 | 0.88 | 2.90 | 0.68 |
| | TWICLLQFAYANRNR | | | | | | | | | |
| | FLY | | | | | | | | | |
| M_38 | AYANRNRFLYIIKLIF | 32 | 96.9% | 67 | 15.8% | 5 | 0.88 | 0.89 | 2.90 | NA |
| | LWLLWPVTLACFVLA | | | | | | | | | |
| | AV | | | | | | | | | |
| ORF1ab_5304 | TSRYWEPEFYEAMYT | 33 | 93.3% | 52 | 43.4% | 8 | 0.59 | 0.93 | 2.89 | NA |
| | PHTVLQAVGACVLCN | | | | | | | | | |
| | SQT | | | | | | | | | |
| S_1205 | KYEQYIKWPWYIWL | 34 | 97.3% | 64 | 29.2% | 2 | 0.81 | 0.82 | 2.89 | NA |
| | GFIAGLIAIVMVTIML | | | | | | | | | |
| | CCM | | | | | | | | | |
| ORF1ab_3732 | SMWALIISVTSNYSG | 35 | 97.1% | 65 | 39.0% | 9 | 0.58 | 0.95 | 2.89 | NA |
| | VVTTVMFLARGIVFM | | | | | | | | | |
| | CVE | | | | | | | | | |
| ORF1ab_1245 | ETKFLTENLLLYIDIN | 36 | 91.8% | 34 | 51.7% | 7 | 0.48 | 0.97 | 2.89 | NA |
| | GNLHPDSATLVSDIDI | | | | | | | | | |
| | T | | | | | | | | | |
| ORF1ab_473 | IAIILASFSASTSAFV | 37 | 76.0% | 29 | 79.5% | 15 | 0.35 | 0.96 | 2.87 | NA |
| | ETVKGLDYKAFKQIVE | | | | | | | | | |
| | S | | | | | | | | | |
| ORF1ab_536 | FASEAARVVRSIFSRT | 38 | 89.7% | 33 | 62.7% | 9 | 0.39 | 0.94 | 2.86 | NA |
| | LETAQNSVRVLQKAA | | | | | | | | | |
| | IT | | | | | | | | | |
| ORF1ab_3698 | RTVYDDGARRVWTL | 39 | 96.7% | 60 | 25.2% | 7 | 0.68 | 0.96 | 2.85 | NA |
| | MNVLTLVYKVYYGN | | | | | | | | | |
| | ALDQA | | | | | | | | | |
| ORF1ab_4241 | MVLGSLAATVRLQA | 40 | 85.3% | 34 | 63.0% | 13 | 0.42 | 0.95 | 2.85 | NA |
| | GNATEVPANSTVLSF | | | | | | | | | |
| | CAFA | | | | | | | | | |
| S_252 | GDSSSGWTAGAAAYY | 41 | 95.7% | 53 | 68.9% | 15 | 0.48 | 0.71 | 2.84 | 0.76 |
| | VGYLQPRTFLLKYN | | | | | | | | | |
| | ENGT | | | | | | | | | |
| ORF1ab_3396 | NFTIKGSFLNGSCGSV | 42 | 90.3% | 43 | 48.3% | 2 | 0.52 | 0.93 | 2.83 | NA |
| | GFNIDYDCVSFCYMH | | | | | | | | | |
| | HM | | | | | | | | | |
| ORF1ab_220 | DFIDTKRGVYCCREH | 43 | 93.5% | 40 | 40.8% | 1 | 0.51 | 0.98 | 2.83 | NA |
| | EHEIAWYTERSEKSY | | | | | | | | | |
| | ELQ | | | | | | | | | |
| ORF1ab_4148 | ALRQMSCAAGTTQT | 44 | 86.3% | 27 | 49.6% | 3 | 0.48 | 0.97 | 2.81 | NA |
| | ACTDDNALAYYNTT | | | | | | | | | |
| | KGGRF | | | | | | | | | |
| S_339 | GEVFNATRFASVYA | 45 | 98.3% | 55 | 36.5% | 8 | 0.69 | 0.77 | 2.81 | 1.42 |
| | WNRKRISNCVADYSV | | | | | | | | | |
| | LYNS | | | | | | | | | |
| ORF1ab_6970 | TEHSWNADLYKLMG | 46 | 86.2% | 49 | 20.4% | 4 | 0.79 | 0.95 | 2.81 | NA |
| | HFAWWTAFVTNVNA | | | | | | | | | |
| | SSSEA | | | | | | | | | |
| S_673 | SYQTQTNSPRRARSV | 47 | 94.5% | 47 | 40.8% | 1 | 0.60 | 0.85 | 2.80 | 1.45 |
| | ASQSIIAYTMSLGAEN | | | | | | | | | |
| | SV | | | | | | | | | |
| S_129 | KVCEFQFCNDPFLGV | 48 | 98.5% | 58 | 39.0% | 9 | 0.60 | 0.83 | 2.80 | 1.14 |
| | YYHKNNKSWMESEF | | | | | | | | | |
| | RVYS | | | | | | | | | |
| ORF3a_33 | ATIPIQASLPFGWLIV | 49 | 96.2% | 54 | 32.7% | 6 | 0.6 | 0.90 | 2.80 | NA |
| | GVALLAVFQSASKIIT | | | | | | | | | |
| | L | | | | | | | | | |
| ORF7A_67 | CPDGVKHVYQLRARS | 50 | 95.0% | 49 | 21.7% | 7 | 0.65 | 0.98 | 2.79 | NA |
| | VSPKLFIRQEEVQEL | | | | | | | | | |
| | YSP | | | | | | | | | |
| ORF1ab_3946 | SEFSSLPSYAAFATA | 51 | 78.5% | 33 | 61.6% | 10 | 0.42 | 0.97 | 2.79 | NA |
| | QEAYEQAVANGDSEV | | | | | | | | | |
| | VLK | | | | | | | | | |
| ORF1ab_593 | NNLVVMAYITGGVV | 52 | 87.9% | 37 | 54.5% | 5 | 0.43 | 0.94 | 2.79 | NA |
| | QLTSQWLTNIFGTVY | | | | | | | | | |
| | EKLK | | | | | | | | | |
| ORF3a_204 | HSYFTSDYYQLYSTQ | 53 | 96.3% | 51 | 42.3% | 9 | 0.47 | 0.92 | 2.78 | NA |
| | LSTDTGVEHVTFFIY | | | | | | | | | |
| | NKI | | | | | | | | | |
| S_53 | DLFLPFFSNVTWFHA | 54 | 85.0% | 34 | 47.2% | 9 | 0.60 | 0.81 | 2.73 | 1.30 |
| | HIVSGTNGTKRFDNP | | | | | | | | | |
| | VLP | | | | | | | | | |
| S_1030 | SECVLGQSKRVDFCG | 55 | 88.8% | 41 | 51.2% | 4 | 0.46 | 0.84 | 2.70 | 1.01 |
| | KGYHLMSFPQSAPHG | | | | | | | | | |
| | WF | | | | | | | | | |
| N_82 | DQIGYYRRATRRIRG | 56 | 95.1% | 45 | 19.0% | 6 | 0.61 | 0.89 | 2.65 | NA |
| | GDGKMKDLSPRWYF | | | | | | | | | |
| | YYLG | | | | | | | | | |
| S_762 | QLNRALTGIAVEQDK | 57 | 81.1% | 35 | 52.6% | 7 | 0.44 | 0.79 | 2.57 | 1.23 |
| | NTQEVFAQVKQIYKT | | | | | | | | | |
| | PPI | | | | | | | | | |
| S_165 | NCTFEYVSQPFLMDL | 58 | 84.3% | 36 | 35.6% | 8 | 0.39 | 0.87 | 2.46 | 1.19 |
| | EGKQGNFKNLREFVF | | | | | | | | | |
| | KNI | | | | | | | | | |
| S_1081 | ICHDGKAHFPREGVF | 59 | 90.9% | 41 | 8.0% | 2 | 0.62 | 0.81 | 2.42 | 1.58 |
| | VSNGTHWFVTQRNF | | | | | | | | | |
| | YEPQ | | | | | | | | | |
| N_305 | AQFAPSASAFFGMSRI | 60 | 87.5% | 40 | 6.4% | 1 | 0.46 | 0.91 | 2.31 | NA |
| | GMEVTPSGTWLTYTG | | | | | | | | | |
| | AI | | | | | | | | | |
| S_490 | FPLQSYGFQPTNGVG | 61 | 89.6% | 36 | 13.4% | 1 | 0.51 | 0.76 | 2.30 | 1.57 |
| | YQPYRVVVLSFELLH | | | | | | | | | |
| | APA | | | | | | | | | |
| S_806 | LPDPSKPSKRSFIEDL | 62 | 79.4% | 23 | 26.5% | 4 | 0.41 | 0.80 | 2.27 | 1.31 |
| | LFNKVTLADAGFIKQY | | | | | | | | | |
| | G | | | | | | | | | |
| S_394 | NVYADSFVIRGDEVR | 63 | 82.9% | 26 | 20.8% | 2 | 0.41 | 0.79 | 2.23 | 1.57 |
| | QIAPGQTGKIADYNY | | | | | | | | | |
| | KLP | | | | | | | | | |
| S_462 | KPFERDISTEIYQAG | 64 | 74.8% | 27 | 18.7% | 5 | 0.51 | 0.77 | 2.21 | 1.29 |
| | STPCNGVEGFNCYFP | | | | | | | | | |
| | QLS | | | | | | | | | |
| S_445 | VGGNYNYLYRLFRKS | 65 | 88.0% | 34 | 10.6% | 4 | 0.42 | 0.77 | 2.18 | 1.69 |
| | NLKPFERDISTEIYQ | | | | | | | | | |
| | AGS | | | | | | | | | |

### II. DEFINITIONS

In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements and components comprising one unit and elements or components that comprise more than one unit unless specifically stated otherwise.

As used herein, the term “about,” when used in conjunction with a percentage or other numerical amount, means plus or minus 10% of that percentage or other numerical amount. For example, the term “about 80%,” would encompass 80% plus or minus 8%.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials define a term in a manner that contradicts the definition of that term in this application, this application controls.

As used herein, and unless otherwise indicated, the terms “disease”, “disorder” or “condition” refer to a state of being or health status of a patient or subject capable of being treated with a compound, pharmaceutical composition, or method provided herein. In some embodiments, the disease is a viral infection (e.g., a SARS-CoV-2 infection).

As used herein, and unless otherwise indicated, the terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease.

As used herein, and unless otherwise indicated, the terms “prevent,” “preventing,” and “prevention” contemplate an action that occurs before a patient begins to suffer from a disorder that involves a viral infection that inhibits or reduces the severity of such viral infection.

As used herein, and unless otherwise specified, a “therapeutically effective amount” of a compound is an amount sufficient to provide any therapeutic benefit in the treatment or prevention of a viral infection, or to delay or minimize one or more symptoms associated with a viral infection. A therapeutically effective amount of a compound means an amount of the compound, alone or in combination with one or more other therapies and/or therapeutic agents that provide any therapeutic benefit in the treatment or management of a viral infection.

As used herein, and unless otherwise specified, an “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of a “therapeutically effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, and unless otherwise specified, a “prophylactically effective amount” of a compound is an amount sufficient to prevent or delay the onset of cancer or one or more symptoms associated with cancer or prevent or delay its recurrence. A prophylactically effective amount of a compound means an amount of the compound, alone or in combination with one or more other treatment and/or prophylactic agent that provides a prophylactic benefit in the prevention of a disease such as a viral infection. The term “prophylactically effective amount” can encompass an amount that prevents a disease such as a viral infection, improves overall prophylaxis, or enhances the prophylactic efficacy of another prophylactic agent. The “prophylactically effective amount” can be prescribed prior to, for example, the development of a disease such as a viral infection.

As used herein, “patient” or “subject in need thereof” refers to a living organism suffering from or prone to a disease or condition that can be treated by administration of a composition or pharmaceutical composition as provided herein. Non-limiting examples include humans, primates, companion animals (dogs, cats, etc.), other mammals, such as but not limited to, bovines, rats, mice, monkeys, goat, sheep, cows, deer, as well as other non-mammalian animals. In some embodiments, a patient is human.

As used herein, the term “conservative substitution” generally refers to amino acid replacements that preserve the structure and functional properties of a protein or polypeptide. Such functionally equivalent (conservative substitution) peptide amino acid sequences include, but are not limited to, additions or substitutions of amino acid residues within the amino acid sequences encoded by a nucleotide sequence that result in a silent change, thus producing a functionally equivalent gene product. Conservative amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY, 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices, and materials similar or equivalent to those described herein can be used in the practice of this disclosure. The following definitions are provided to facilitate understanding of certain terms used frequently herein and are not meant to limit the scope of the present disclosure.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” as used herein, refers to a cell carrying out metabolic or other functions sufficient to preserve or replicate its genomic DNA. A cell can be identified by well-known methods in the art including, for example, the presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example, mammalian, insect (e.g., Spodoptera) and human cells. Cells may be useful when they are naturally non-adherent or have been treated not to adhere to surfaces, for example by trypsinization.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Nucleic acid” refers to deoxyribonucleotides or ribonucleotides and polymers thereof, in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single- and double-stranded DNA, single- and double-stranded RNA (including siRNA), and hybrid molecules having mixtures of single- and double-stranded DNA and RNA. Nucleic acid as used herein also refers to nucleic acids that have the same basic chemical structure as a naturally occurring nucleic acid. Such analogs have modified sugars and/or modified ring substituents but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogs include, without limitation, phosphorothioates, phosphoramidites, methyl phosphonates, chiral-methyl phosphonates, 2-O-methyl ribonucleotides, and peptide-nucleic acids (PNAs).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the disclosure or individual domains of the polypeptides of the disclosure), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more particularly over a region that is 100 to 500 or 1000 or more nucleotides in length. The present disclosure includes polypeptides that are substantially identical to any identified herein.

The word “expression” or “expressed” as used herein in reference to a gene means the transcriptional and/or translational product of that gene. The level of expression of a DNA molecule in a cell may be determined on the basis of either the amount of the corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 MOLECULAR CLONING: A LABORATORY MANUAL, 18.1-18.88. Expression of a transfected gene can occur transiently or stably in a cell. During “transient expression” the transfected gene is not transferred to the daughter cell during cell division.

Since its expression is restricted to the transfected cell, expression of the gene is lost over time. In contrast, stable expression of a transfected gene can occur when the gene is co-transfected with another gene that confers a selective advantage to the transfected cell. Such a selective advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced into a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetization and electroporation. In some embodiments, the nucleic acid molecules are introduced into a cell using electroporation following standard procedures are well known in the art. For viral-based methods of transfection, any useful viral vector may be used in the methods described herein. Examples of viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) and Prochiantz (2007).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Typically, the antigen-binding region of an antibody plays a significant role in determining the specificity and affinity of binding. In some embodiments, antibodies or fragments of antibodies may be derived from different organisms, including humans, mice, rats, hamsters, camels, etc. Antibodies may include antibodies that have been modified or mutated at one or more amino acid positions to improve or modulate a desired function of the antibody (e.g., glycosylation, expression, antigen recognition, effector functions, antigen binding, specificity, etc.).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions typically requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high-performance liquid chromatography. A protein that is the predominant species present in a preparation is substantially purified.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

### III. IMMUNE THERAPIES INVOLVING VACCINES

A. Vaccines

Vaccines are a form of active immunotherapy where an antigenic peptide, polypeptide or protein, such as the antigens disclosed in Table 4, is administered to a subject. Vaccines may be administered systemically, such as intranvenously, intramuscularly, or intradermally. Vaccines may also be administered multiple times to enhance the immune response against the administered antigens.

1. Adjuvants

In one embodiment, adjuvant may be a T helper epitope, such as a universal T helper epitope. A universal T helper epitope as used herein refers to a peptide or other immunogenic molecule, or a fragment thereof, that binds to a multiplicity of MHC class II molecules in a manner that activates T-cell function in a class II (CD4+ T cells)-restricted manner. In another embodiment, the T helper epitope may be a universal T helper epitope such as PADRE (pan-DR epitope) comprising the peptide sequence AKXVAAWTLKAAA (SEQ ID NO: 75), wherein X may be cyclohexylalanyl. PADRE specifically has a CD4+ T-helper epitope, that is, it stimulates induction of a PADRE-specific CD4+ T helper response. Tetanus toxoid has T helper epitopes that work in the similar manner as PADRE. Tetanus and diphtheria toxins have universal epitopes for human CD4+ cells. (Diethelm-Okita, B. M. et al., Universal epitopes for human CD4+ cells on tetanus and diphtheria toxins. J. Infect. Diseases, 181:1001-1009, 2000). In another embodiment, the T helper epitope may be a tetanus toxoid peptide such as F21E comprising the peptide sequence FNNFTVSFWLRVPKVSASHLE (SEQ ID NO: 76) (amino acids 947-967). In some embodiments, the vaccines can also include IL-12, IL-15, IL-28, and/or RANTES.

As also well known in the art, the immunogenicity of a particular immunogen composition can be enhanced by the use of non-specific stimulators of the immune response, known as adjuvants. Adjuvants have been used experimentally to promote a generalized increase in immunity against poorly immunogenic antigens (e.g., U.S. Pat. No. 4,877,611). Immunization protocols have used adjuvants to stimulate responses for many years, and as such adjuvants are well known to one of ordinary skill in the art. Some adjuvants affect the way in which antigens are presented. For example, the immune response is increased when protein antigens are adsorbed to alum. Emulsification of antigens also prolongs the duration of antigen presentation and initiates an innate immune response. Suitable molecule adjuvants include all acceptable immunostimulatory compounds, such as cytokines, toxins or synthetic compositions.

In some aspects, the compositions described herein may further comprise another adjuvant. Although Alum is an approved adjuvant for humans, adjuvants in experimental animals include complete Freund's adjuvant (a non-specific stimulator of the immune response containing killed Mycobacterium tuberculosis), incomplete Freund's adjuvants and aluminum hydroxide adjuvant. Other adjuvants that may also be used in animals and sometimes humans include Interleukin (IL)-1, IL-2, IL-4, IL-7, IL-12, interferon, Bacillus Calmette-Guérin (BCG), aluminum hydroxide, muramyl dipeptide (MDP) compounds, such as thur-MDP and nor-MDP (N-acetylmuramyl-L-alanyl-D-isoglutamine MDP), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used.

In one aspect, and approved for humans, an adjuvant effect is achieved by use of an agent, such as alum, used in about 0.05 to about 0.1% solution in phosphate buffered saline. Alternatively, in experimental animals the antigen is made as an admixture with synthetic polymers of sugars (Carbopol®) used as an about 0.25% solution. Adjuvant effects may also be achieved by aggregation of the antigen in the vaccine by heat treatment with temperatures ranging between about 70° to about 101° C. for a 30 second to 2-minute period, respectively. Aggregation by reactivating with pepsin treated (Fab) antibodies to albumin, mixture with bacterial cell(s) such as C. parvum, an endotoxin or a lipopolysaccharide component of Gram-negative bacteria, emulsion in physiologically acceptable oil vehicles, such as mannide mono-oleate (Aracel A), or emulsion with a 20% solution of a perfluorocarbon (Fluosol-DA®) used as a block substitute, also may be employed.

Some adjuvants, for example, certain organic molecules obtained from bacteria, act on the host rather than on the antigen. An example is MDP, a bacterial peptidoglycan. The effects of MDP, as with most adjuvants, are not fully understood, although it is now beginning to be understood that they activate cells of the innate immune system, e.g. dendritic cells, macrophages, neutrophils, NKT cells, NK cells, etc. MDP stimulates macrophages but also appears to stimulate B cells directly. The effects of adjuvants, therefore, are not antigen-specific. If they are administered together with a purified antigen, however, they can be used to selectively promote the response to the antigen.

In certain embodiments, hemocyanins and hemoerythrins may also be used in the compositions of the present disclosure. The use of hemocyanin from keyhole limpet (KLH) is used in certain embodiments, although other molluscan and arthropod hemocyanins and hemoerythrins may be employed.

Various polysaccharide adjuvants may also be used. For example, the use of various pneumococcal polysaccharide adjuvants on the antibody responses of mice has been described. The doses that produce optimal responses, or that otherwise do not produce suppression, should be employed as indicated. Polyamine varieties of polysaccharides are particularly contemplated, such as chitin and chitosan, including deacetylated chitin.

Another group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative muramyl peptide phosphatidylethanolamide (MTPPE) are also contemplated.

U.S. Pat. No. 4,950,645 describes a lipophilic disaccharide-tripeptide derivative of muramyl dipeptide which is described for use in artificial liposomes formed from phosphatidyl choline and phosphatidyl glycerol. This is effective in activating human monocytes and destroying tumor cells, but is non-toxic in generally high doses. The compounds of U.S. Pat. No. 4,950,645 and PCT Patent Application WO 91/16347, are contemplated for use with cellular carriers and other embodiments of the present disclosure.

BCG and BCG-cell wall skeleton (CWS) may also be used as adjuvants, with or without trehalose dimycolate. Trehalose dimycolate may be used itself. Trehalose dimycolate administration has been shown to correlate with augmented resistance to influenza virus infection in mice (Azuma et al., 1988). Trehalose dimycolate may be prepared as described in U.S. Pat. No. 4,579,945. BCG is an important clinical tool because of its immunostimulatory properties. BCG acts to stimulate the reticuloendothelial system (RES), activates natural killer (NK) cells and increases proliferation of hematopoietic stem cells. Cell wall extracts of BCG have proven to have excellent immune adjuvant activity. Molecular genetic tools and methods for mycobacteria have provided the means to introduce foreign genes into BCG. Live BCG is an effective and safe vaccine used worldwide to prevent tuberculosis. BCG and other mycobacteria are highly effective adjuvants, and the immune response to mycobacteria has been studied extensively. With nearly 2 billion immunizations, BCG has a long record of safe use in man. It is one of the few vaccines that can be given at birth, it engenders long-lived immune responses with only a single dose, and there is a worldwide distribution network with experience in BCG vaccination. An exemplary BCG vaccine is sold as TICE BCG (Organon Inc., West Orange, N.J.).

Amphipathic and surface-active agents, e.g., saponin and derivatives such as QS21 (Cambridge Biotech), form yet another group of adjuvants for use with the immunogens of the present disclosure. Nonionic block copolymer surfactants may also be employed. Oligonucleotides are another useful group of adjuvants. Quil A and lentinen are other adjuvants that may be used in certain embodiments of the present disclosure.

Another group of adjuvants are the detoxified endotoxins, such as the refined detoxified endotoxin of U.S. Pat. No. 4,866,034. These refined detoxified endotoxins are effective in producing adjuvant responses in mammals. Of course, the detoxified endotoxins may be combined with other adjuvants to prepare multi-adjuvant-incorporated cells. For example, combination of detoxified endotoxins with trehalose dimycolate is particularly contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cCWS or CWS and trehalose dimycolate, as described in U.S. Pat. Nos. 4,436,727, 4,436,728 and 4,505,900. Combinations of just CWS and trehalose dimycolate, without detoxified endotoxins, are also envisioned to be useful, as described in U.S. Pat. No. 4,520,019.

Those of skill in the art will know the different kinds of adjuvants that can be conjugated to vaccines in accordance with this disclosure and which are approved for human vs experimental use. These include alkyl lysophosphilipids (ALP); BCG; and biotin (including biotinylated derivatives) among others. Certain adjuvants particularly contemplated for use are the teichoic acids from Gram⁻ bacterial cells. These include the lipoteichoic acids (LTA), ribitol teichoic acids (RTA) and glycerol teichoic acid (GTA). Active forms of their synthetic counterparts may also be employed in connection with the compositions of this disclosure.

Various adjuvants, even those that are not commonly used in humans, may still be employed in animals. Adjuvants may be encoded by a nucleic acid (e.g., DNA or RNA). It is contemplated that such adjuvants may be also be encoded in a nucleic acid (e.g., an expression vector) encoding the antigen, or in a separate vector or other construct. Nucleic acids encoding the adjuvants can be delivered directly, such as for example with lipids or liposomes.

2. Biological Response Modifiers (BRM)

In addition to adjuvants, it may be desirable to co-administer BRM, which have been shown to upregulate T cell immunity or downregulate suppressor cell activity. Such BRMs include, but are not limited to, cimetidine (CIM; 1200 mg/d) (Smith/Kline, PA); low-dose cyclophosphamide (CYP; 300 mg/m²) (Johnson/Mead, NJ), cytokines such as interferon, IL-2, or IL-12 or genes encoding proteins involved in immune helper functions, such as B-7. Additional biological response modifiers include those described in Gupta and Kanodia, 2002 and Bisht, et al., 2010, both of which are incorporated herein by reference.

3. Chemokines

Chemokines, nucleic acids that encode for chemokines, and/or cells that express such also may be used as vaccine components. Chemokines generally act as chemoattractants to recruit immune effector cells to the site of chemokine expression. It may be advantageous to express a particular chemokine coding sequence in combination with, for example, a cytokine coding sequence, to enhance the recruitment of other immune system components to the site of treatment. Such chemokines include, for example, RANTES, MCAF, MIP1-α, MIP1-β, IP-10 and combinations thereof. The skilled artisan will recognize that certain cytokines are also known to have chemoattractant effects and could also be classified under the term chemokines.

4. Immunogenic Carrier Proteins

In some embodiments, the vaccine antigens described herein may be chemically coupled to a carrier or recombinantly expressed with a immunogenic carrier peptide or polypetide (e.g., an antigen-carrier fusion peptide or polypeptide) to enhance an immune reaction. Exemplary immunogenic carrier amino acid sequences include hepatitis B surface antigen (HBSA), tetanus toxoid (TT), keyhole limpet hemocyanin (KLH) and BSA. In humans, TT would be advantageous since it is already an approved protein vaccine. For experimental animals, other albumins such as OVA, mouse serum albumin or rabbit serum albumin also can be used as immunogenic carrier proteins. Means for conjugating a polypeptide or peptide to an immunogenic carrier protein are well known in the art and include, for example, glutaraldehyde, m-maleimidobenzoyl-N-hydroxy succinimide ester, carbodiimide and bis-biazotized benzidine.

5. Engineered Dendritic Cells

In some embodiments, the disclosure relates to dendritic cell (DC) vaccines. DC vaccines include antigen-presenting cells that are able to induce specific T cell immunity, which are harvested from the patient or from a donor. The DCs can then be exposed in vitro to a peptide antigen from Table 4, for which T cells are to be generated in the patient. Dendritic cells loaded with the antigen are then injected back into the patient. Immunization may be repeated multiple times if desired. Methods for harvesting, expanding, and administering dendritic cells are well known in the art, for example, as described in Fong et al. (2001). DC vaccines are further described elsewhere, such as in U.S. Pat. No. 7,939,059; U.S. Pat. Publn. 2005/0238626; and U.S. Pat. Publn. 2007/0020238, each of which is incorporated herein by reference in its entirety. Typical doses of DCs administered to the patient include at least about 10 million cells.

6. MHC Class I Antigens

For an MHC class I peptide to trigger (elicit) a cellular immune response, it also must bind to an MHC-molecule. This process is dependent on the allele of the MHC-molecule and specific polymorphisms of the amino acid sequence of the peptide. Thus, when considering vaccines of this nature, matching of MHC-antigen profiles to the MHC profile of the patient is important.

MHC-class-I-binding peptides are usually 8-12 amino acid residues in length and usually contain two conserved residues (“anchors”) in their sequence that interact with the corresponding binding groove of the MHC-molecule. In this way each MHC allele has a “binding motif” determining which peptides can bind specifically to the binding groove. In the MHC class I dependent immune reaction, peptides not only have to be able to bind to certain MHC class I molecules expressed by tumor cells, they subsequently also have to be recognized by T cells bearing specific T cell receptors (TCR).

### IV. METHODS OF USE

In some embodiments, the present disclosure provides methods for immunotherapy comprising administering an effective amount of the vaccine of the present disclosure. In one embodiment, a medical disease or disorder is treated by eliciting an immune response. In certain embodiments of the present disclosure, a viral infection is prevented by eliciting a protective immune response.

In certain embodiments of the present disclosure, a vaccine is delivered to an individual in need thereof, such as an individual that is at risk for exposure to SARS-CoV-2.

The vaccine then enhances the individual's immune system to attack the virus. In some cases, the individual is provided with one or more doses of the vaccine. In cases where the individual is provided with two or more doses of the vaccine, the duration between the administrations should be sufficient to allow time for propagation in the individual, and in specific embodiments the duration between doses is 1, 2, 3, 4, 5, 6, 7, 8, 9, 10, 11, 12, 13, 14, 15, 16, 17, 18, 19, 20, 21, 22, 23, 24, 25, 26, 27, or 28 or more days.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of a vaccine can be administered by a number of routes, including parenteral administration, for example, by intravenous, intraperitoneal, intramuscular, intrasternal, intradermal, or intraarticular injection, or by infusion.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

The combination therapies of the present invention may also find use in further combinations. Effective combination therapy may be achieved with a single composition or pharmacological formulation that includes multiple agents, or with multiple compositions or formulations, administered at the same time, wherein one composition includes a combination described elsewhere herein, and the other includes the second agent(s). Alternatively, the therapy may precede or follow the other agent treatment by intervals ranging from minutes to months.

Various combinations may be employed, such as when a vaccine described elsewhere herein is “A” and “B” represents a secondary agent, non-limiting examples of which are described below:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

It is contemplated that other therapeutic agents may be used in conjunction with the vaccines of the current invention. In some embodiments, the present invention contemplates the use of one or more other therapies for the treatment of COVID-19 include the use of a SARS-CoV-2 protease inhibitor, anti-platelet drugs, an anti-coagulation agent, a human type I interferon, a corticosteroid, or remdesivir.

In some embodiments, the anti-platelet drug is aspirin, an ADP receptor antagonist (e.g., ticlopidine, clopidogrel, cangrel or, prasugrel, ticagrelor, thienopyridine), or a glycoprotein IIb/IIIa receptor inhibitor (e.g., abciximab, eptifibatide, ticofiban). In some embodiment, the anti-coagulation agent is rivaroxaban, apixaban, dipyridamole, cilostazol, atromentin, edoxaban, fondaprinux, betrixaban, letaxaban, eribaxaban, hirudin, a thrombin inhibitor (e.g., lepirudin, desirudin, dabigatran, bivalirudin, ximelagatran), argatroban, batroxobin, hementin, low molecular weight heparin, unfractionated heparin, vitamin E, or a vitamin K antagonist (e.g., warfarin (Coumadin), acenocoumarol, phenprocoumon, phenindione).

Human type I interferons (IFNs) are a large subgroup of interferon proteins that help regulate the activity of the immune system. The mammalian types are designated IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin). Type I interferons have shown efficacy against the replication of various viruses, included Zika virus, chikungunya virus, flaviviruses, and hepatitis C virus. “Interferon compounds” include interferon-alpha, interferon-alpha analogues, interferon-alpha derivatives, interferon-alpha conjugates, interferon beta, interferon-beta analogues, interferon-beta derivatives, interferon-beta conjugates and mixtures thereof. The whole protein or its fragments can be fused with other peptides and proteins such as immunoglobulins and other cytokines. Interferon-alpha and interferon-beta conjugates may represent, for example, a composition comprising interferon-beta coupled to a non-naturally occurring polymer comprising a polyalkylene glycol moiety. Preferred interferon compounds include Roferon®, Intron®, Alferon®, Infergen®, Omniferon®, Alfacon-1, interferon-alpha, interferon-alpha analogues, pegylated interferon-alpha, polymerized interferon-alpha, dimerized interferon-alpha, interferon-alpha conjugated to carriers, interferon-alpha as oral inhalant, interferon-alpha as injectable compositions, interferon-alpha as a topical composition, Roferon® analogues, Intron® analogues, Alferon® analogues, and Infergen® analogues, Omniferon® analogues, Alfacon-1 analogues, interferon beta, Avonex™, Betaferon™, Betaferon™, Rebif™, interferon-beta analogues, pegylated interferon-beta, polymerized interferon-beta, dimerized interferon-beta, interferon-beta conjugated to carriers, interferon-beta as oral inhalant, interferon-beta as an injectable composition, interferon-beta as a topical composition, Avonex™analogues, Betaferon™ Betaferon™ analogues, and Rebif™ analogues. Alternatively, agents that induce interferon-alpha or interferon-beta production or mimic the action of interferon-alpha or interferon-beta may also be employed. Interferon inducers include tilorone, poly(I)-poly(C), imiquimod, cridanimod, bropirimine.

### V. ARTICLES OF MANUFACTURE OR KITS

An article of manufacture or a kit is provided comprising compositions for SARS-CoV-2 vaccination. The article of manufacture or kit can further comprise a package insert comprising instructions for using the vaccine. Any of the vaccine compositions described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. Suitable containers for the one or more agent include, for example, bottles, vials, bags and syringes.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1— Methods

Population-scale HLA Class I & II Presentation. We identified potential SARS-CoV-2 epitopes by applying our recently published algorithm for scoring population-scale HLA presentation of tumor driver gene, to the SARS-CoV-2 genome (GenBank Acc #: MN908947.3) (Yarmarkovich et al., 2020). All possible 33mer amino acid sequences covering every 9mer peptide from the 10 SARS-CoV-2 genes were generated and we employed netMHC-4.0 to predict the binding affinities of each viral peptide across 84 HLA class I alleles. We considered peptides with binding affinities <500 nM putative epitopes. MHC class II binding affinities were predicted as previously described across 36 HLA class II alleles population using netMHCII 2.3.

The frequencies of HLA class I alleles-A/B/C and HLA class II alleles-DRB1/3/4/5 were obtained from Be the Match bone marrow registry (Gragert et al., 2013). HLA class II alleles-DQA1/DQB1 and -DPA1/DPB1 were obtained from (Sidney et al., 2010) and (Solberg et al., 2008), respectively.

Conservation Scoring. We obtained all 1,024 unique protein sequences categorized by each of the 10 SARS-CoV-2 genes available from the NCBI as of 25 Mar. 2020. All sequences were aligned using Clustal Omega (Sievers et al., 2011) and each position summed for homology. In addition to human sequences, we scored each amino acid position for homology across 15 species of related coronavirus found in bats, pigs, camels, mice, and humans (SARS-CoV, SARS-CoV-2, and MERS). Each amino acid was scored up to 100% conservation. 33mer peptides were then scored in Equation 1:

$$C = \frac{{\sum }_{1}^{33} {A}_{i} - Y}{Z - Y} \qquad [ 1 ]$$

where C is the 33mer conservation score, A is the conservation percentage of an amino acid position, Y is the minimum 33mer conservation percentage sum, and Z is the maximum 33mer conservation percentage sum. In the same way, we ranked the conservation across 274 SARS-CoV-2 amino acid sequences available at the time of this study. A final conservation score was generated by averaging the conservation scores from cross-species and interhuman variation and 33mer peptides with the highest score were considered the most conserved.

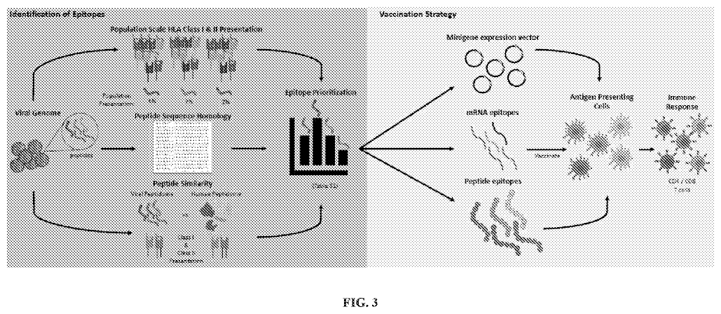

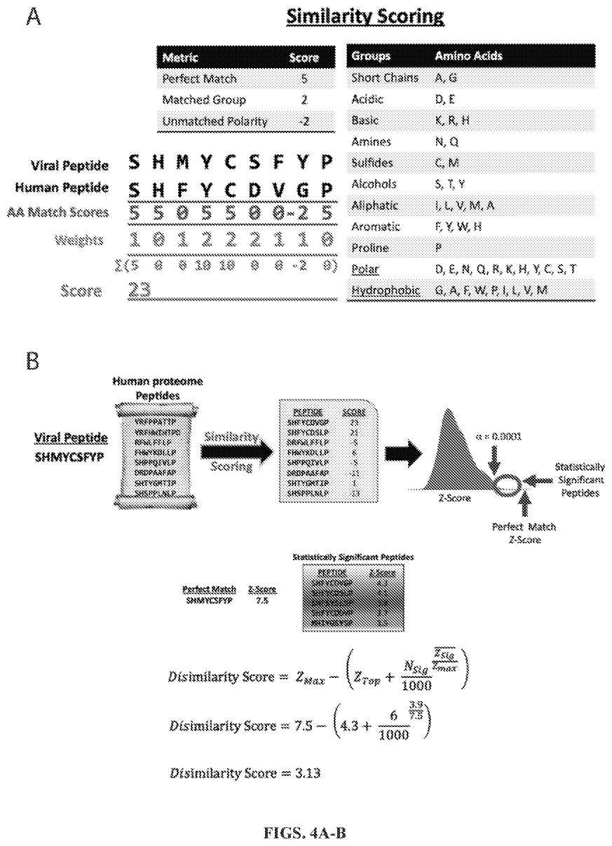

Dissimilarity Scoring. 3,524 viral epitopes were compared against the normal human proteome on each of their MHC binding partners, testing a total of 12, 383 peptide/WIC pairs against the entire human proteome (85,915,364 normal peptides across HLAs), assigning a similarity score for each peptide. Residues in the same position of the viral and human peptides with a perfect match, similar amino acid classification, or different polarity, were assigned scores of five, two, or negative two respectively. Similarity scores were calculated based on amino acid classification and hydrophobicity were determined using residues one and three through eight, and excluding WIC anchor residues (FIG. 4A). The canonical TCR-interaction hotspots (residues four through six) were double weighted (Gagnon et al., 2005; Gras et al., 2009; Ishizuka et al., 2008). The similarity scores generated for each viral peptide were converted to Z-scores and peptides with a p<0.0001 were selected for comparison to viral epitopes (FIG. 4B). The overall dissimilarity score for the viral peptide was then calculated using Equation 2:

$${S}_{S i m} = {Z}_{Max} - ( {Z}_{Top} + {\frac{{N}_{Sig}}{1000}}^{\frac{\overline{{Z}_{Sig}}}{{Z}_{max}}} ) \qquad [ 2 ]$$

where S_{Sim} is the overall dissimilarity score for the viral peptide, Z_{Max} is the highest possible Z-score given a perfect sequence match to the viral peptide, Z_{Top} is the highest Z-score from the human proteome, N_{Sig} is the number of statistically significant peptides from the human proteome, and Z̅_̅{̅S̅i̅g̅}̅ is the mean Z-score from the statistically significant peptides given a p<0.001.

B cell Epitope Scoring. We used BepiPred 2.0 and DiscoTope 2.0 (Jespersen et al., 2017; Kringelum et al., 2012) to score individual amino acid residues, assessing linear epitopes in Matrix, Envelope, and Spike proteins, and conformational epitopes for Spike protein, based on published structure (PDB 6VYB). We summed and normalized linear and conformational, using separate normalizations for proteins in which only linear predictions were available.

### Example 2— Results

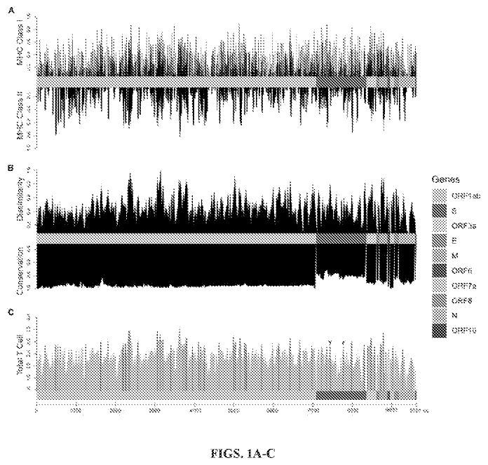

We used our recently published methods for scoring population-scale HLA presentation of individual putative cancer antigens along the length of a protein to analyze the population-scale HLA presentation of individual peptides derived from all 10 SARS-CoV-2 genes across 84 Class I HLA alleles (Yarmarkovich et al., 2020), representing 99.4% of the population represented in the Bone Marrow Registry (Gragert, Madbouly, Freeman, & Maiers, 2013). We identified 3,524 SARS-CoV-2 epitopes that are predicted to bind at least one HLA class I allele, with peptide FVNEFYAYL (SEQ ID NO: 77) capable of binding 30 unique HLA alleles representing 90.2% of the US population (FIG. 1A, top; Table 1). We tested various epitope sizes to maximize HLA presentation across the viral proteome, finding that 33 amino acid epitopes generated maximal population-scale HLA presentation, and suggest that these 33mers can be expressed in a multicistronic construct in dendritic cells to induce potent immune response across the vast majority of the population (An, Rodriguez, Harkins, Zhang, & Whitton, 2000; Lu et al., 2014). We identified areas predicted to be presented across the majority of the population, including a single 33mer ISNSWLMWLIINLVQMAPISAMVRMYIFFASFY (SEQ ID NO: 78) containing epitopes capable of binding 82 of the 84 HLAs alleles studied here.

As it has been shown that presentation by both Class I and Class II MHC is necessary for robust memory B and T cell responses (Alspach et al., 2019; McHeyzer-Williams et al., 2012), we next analyzed presentation of these viral epitopes on 36 MHC Class II HLA alleles, representing 92.6% of the population (FIG. 1A, bottom; Table 1). Peptides derived from the 33mer IAMSAFAMMFVKHKHAFLCLFLLPSLATVAYFN (SEQ ID NO: 1) were presented on 24 HLA class II alleles, representing 82.1% of US population, and peptides from the same epitope were predicted to be presented on 74 HLA class I alleles with a population frequency of 98.6%. As HLA frequencies vary based on the composition of each population, the frequency of individual HLA alleles can be adjusted based on specific populations using a SARs-CoV-2 immunogenicity map (see Table 51 of Yarmarkovich et al., Cell. Rep. Med., 1(3):100036, 2020, which is incorporated herein by reference in its entirety).

Next, we sought to identify the most highly conserved regions of the SARS-CoV-2 virus, positing that non-conserved regions that are not involved in newly acquired increased infectivity may be prone to T cell evasion through mutation of MHC-presented epitopes. To do this, we compared the amino acid sequence of SARS-CoV-2 to fourteen Coronaviridae family sequences derived from bats, pigs, and camels, scoring each amino acid for conservation across the viral strains. We also scored the conservation across the 1,024 SARS-CoV-2 virus sequences available at the time of this analysis, equally weighing contributions from cross-species and interhuman variation (scores normalized to 0-1, with entirely conserved regions scoring 1). As expected, evolutionary divergence was greatest in the tropism-determining Spike protein and lowest in ORF lab which contains 16 proteins involved in viral replication (FIG. 1B, bottom).

We then compared predicted viral MHC-presented epitopes to self-peptides presented normally on 84 HLA alleles across the entire human proteome from UniProt, prioritizing antigens that are most dissimilar from self-peptides based on: 1) higher predicted safety based on less likelihood of inducing autoimmunity due to cross-reactivity with similar self-peptides presented on WIC; and 2) higher immunogenicity of dissimilar peptides based on an expected greater repertoire of antigen-specific T cells due to lower degree of negative thymic selection. To do this, we compared 3,524 viral epitopes against the normal human proteome on each of their MHC binding partners, testing a total of 12,383 peptide/WIC pairs against the entire human proteome (85,915,364 normal peptides across HLAs), assigning a similarity score for each peptide, with high scoring peptides representing the highest degree of dissimilarity as compared to the space of all possible WIC epitopes derived from the normal proteome (Methods; FIG. 1B, bottom).

To assign an overall score for T cell antigens, we normalized each of our four scoring parameters (represented in FIGS. 1A and 1B) between 0-1 and summed each metric to obtain a final epitope score, highlighting the local maxima of epitopes scoring in the 90^{th} percentile, highlighting 55 top scoring T cell epitopes across 9 SARS-CoV-2 genes as epitopes for vaccination (FIG. 1C, Table 2).

Finally, we sought to characterize B cell epitopes, assessing linear epitopes in Spike (S), Matrix (M), and Envelope (E) proteins which are exposed and expected to be accessible to antibodies, and characterized conformational epitopes in the Spike protein for which structural data are available using BepiPred 2.0 and DiscoTope 2.0 (Jespersen, Peters, Nielsen, & Marcatili, 2017; Kringelum, Lundegaard, Lund, & Nielsen, 2012). There was a strong concordance between linear epitope scores and conformational epitope scores (p≤2e⁻¹⁶). We next performed an agnostic scoring of individual amino acid residues in S, M, and E proteins (FIG. 1D), and then used these scores to generate scores for 33mer epitopes along the length of the protein (FIG. 1E). The 33mer VGGNYNYLYRLFRKSNLKPFERDISTEIYQAGS (SEQ ID NO: 65) derived from S protein at position 445 ranked the highest based on combined linear and conformational B cell epitope scoring. We combined T cell epitope scores calculated above with available B cell epitope scores derived from the S, M, and E genes, providing a list of antigens predicted to stimulate both humoral and cellular adaptive immunity (FIG. 1F, Table 4).

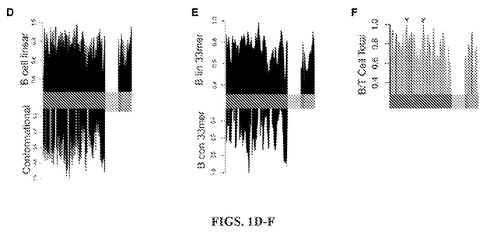

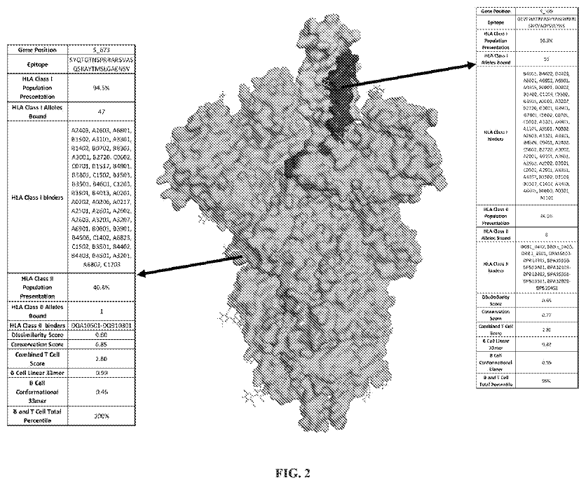

In addition to prioritizing evolutionarily conserved regions, we sought to specifically target acquired vulnerabilities in SARS-CoV-2 by focusing on novel features of this coronavirus that have been shown to contribute to its increased infectivity. The receptor binding domain of the SARS-CoV-2 Spike protein has been reported to have 10-fold higher binding affinity to ACE2 (Wrapp et al., 2020). We show that viral epitope GEVFNATRFASVYAWNRKRISNCVADYSVLYNS (SEQ ID NO: 45) derived from the receptor binding domain (RBD) of the Spike protein (position 339-372) scores in the 90.9^{th} percentile of T epitopes and is the #3 of 1,546 epitopes scored in the S, E, and M genes for combined B and T cell epitopes, with presentation by MHC class I in 98.3% of the population (FIGS. 1C, 1F & 2). Additionally, a novel furin cleavage site has been reported in the SARS-CoV-2 virus, resulting in increased infectivity (Wrapp et al., 2020). Indeed, we find that the epitope SYQTQTNSPRRARSVASQSIIAYTMSLGAENSV (SEQ ID NO: 47) containing the RRAR furin cleavage site of the spike protein ranks in the 90.7^{th} percentile of T cell epitopes and ranks first of 1546 in combined B and T cell epitope, (FIGS. 1C, IF & 2), thereby targeting an additional evolutionary adaptation of SARS-CoV-2. Based on a recently published study identifying receptor binding hotspots deduced by comparing structures of ACE2 bound to the Spike protein from SARS-CoV-2 as compared to SARS-CoV (Shang et al., 2020), we searched for epitopes containing the five acquired residues that increase Spike binding to ACE2, identifying KPFERDISTEIYQAGSTPCNGVEGFNCYFPLQS (SEQ ID NO: 64) as the highest ranked epitope containing all of these residues (hotspots underlined; Table 1). Finally, it is known that mRNA transcripts proximal to the 3′ end of the Coronaviridae family genome show higher abundance consistent with the viral replication process, with S, E, M, and N genes shown to have significantly higher translational efficiency compared to the 5′ transcripts (Cheng, Lau, Woo, & Yuen, 2007; Hiscox, Cavanagh, & Britton, 1995; Irigoyen et al., 2016). We therefore posit that viral epitopes derived from 3′ terminus including the S, E, M, and N genes will have a higher representation on MHC and suggest their prioritization in a vaccine construct. Tables 1-4 and FIG. 2 show the viral epitopes we suggest prioritizing for vaccine development.

Two or more of the viral epitopes presented in Table 4 can be joined to form a linear vaccine construct with a linker present between each epitope. In order to design the linear construct, algorithms are applied to identify immunogenic epitopes arising from junctions. Linkers are chosen to prevent the formation of junctional epitopes having non-specific immunogenicity while also facilitating immune processing of the antigens. Exemplary linkers include GPGPG (SEQ ID NO: 79), AAY, HEYGAEALERAG (SEQ ID NO: 80), and EAAAK (SEQ ID NO: 81). Three signal peptides can be used to traffic constructs to ER, lysosome, and secretion to stimulate MHC class I, MHC class II, and B cell response, respectively.

Briefly, an algorithm was used to minimize immunogencitiy at the 33mer junctions and to order the 33mers and use the appropriate linkers such as to minimize off-target immunogenicity. The algorithm was trained using a matrix of all 65 prioritized 33mers followed by each of the other 64 33mers with each possible linker peptide in between them. Population-scale HLA presentation was calculated for each potential peptide that can arise at each junction, and each 33mer pair was given a total score summing the population-scale presentation of each peptide presented at the junction. The algorithm then optimized the list of 33mers for inclusion in a given construct for minimal total junction immunogenicity along the entire construct.

The top sets of 33mers were put into vectors containing a PADRE adjuvant. DNA vaccines were made containing either only spike epitopes (see SEQ ID NOS: 69-71), or combined epitopes from all conserved regions of the virus (SEQ ID NOS: 72-74), or a vaccine based on T cell epitopes alone (SEQ ID NOS: 66-68). These combinations of 33mers were put into the pVax vector (see e.g., FIG. 5) and electroporated in transgenic mice expressing human HLA-A*02:01.

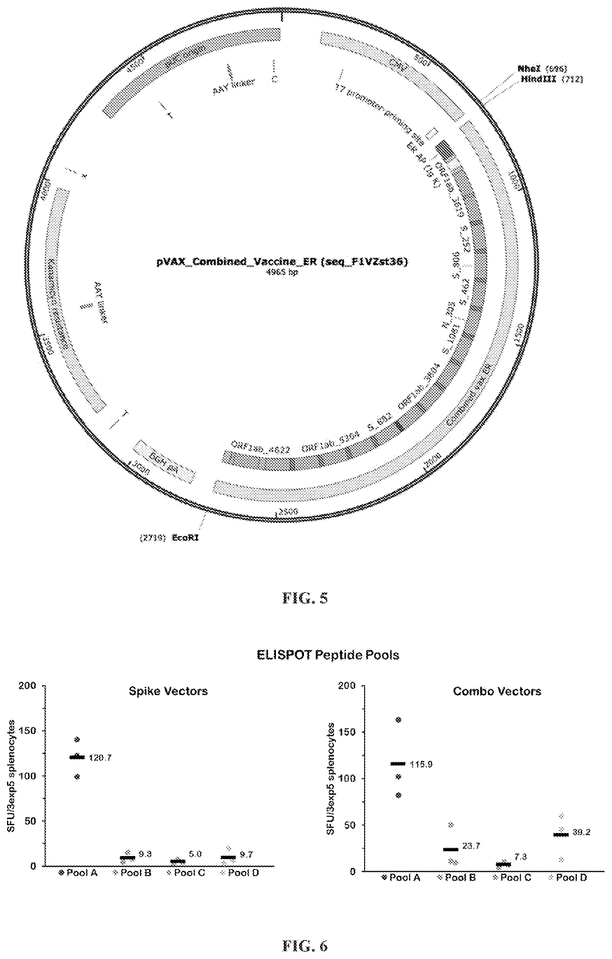

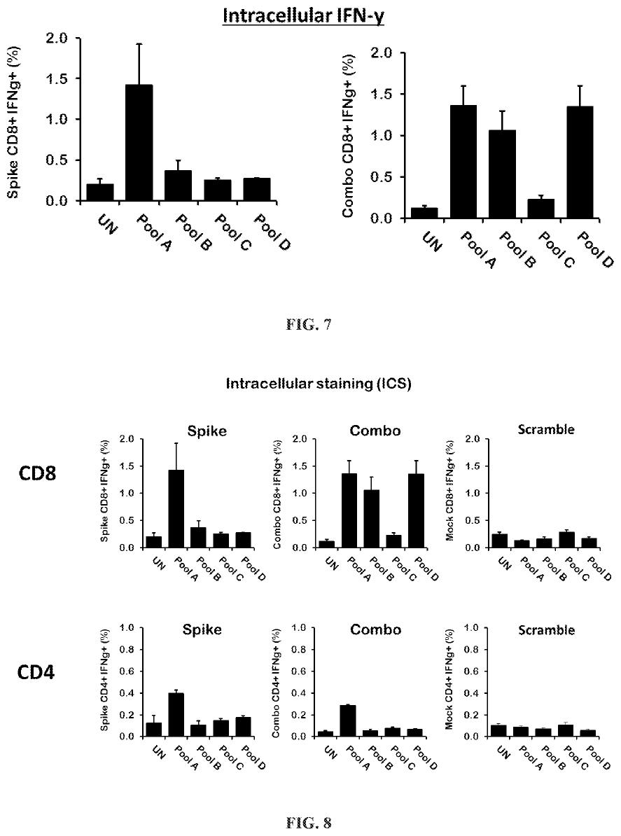

The experiments used a set of overlapping peptide pools covering the span of the construct, measuring cytokine release attributed to each region of the vaccine constructs by ELISPOT. 15mer peptides overlapping by 5aa spanning the length of each construct were synthesized and split into four pools covering each ¼^{th} of the construct in order. Peptide pools were added to splenocytes collected from vaccinated transgenic mice expressing human HLA-A*02:01 and spots counted for each mouse (represented by each dot). Splenocytes stimulated by peptides in pool A in spike vector shows significant IFN-γ production and by pools A, B, and D in the combination vector (FIG. 6). IFN-γ is upregulated in CD8 T cells pulsed with pool A peptides in spike vaccine and in pools A, B, and D in combined vector, and not in controls (FIG. 7).

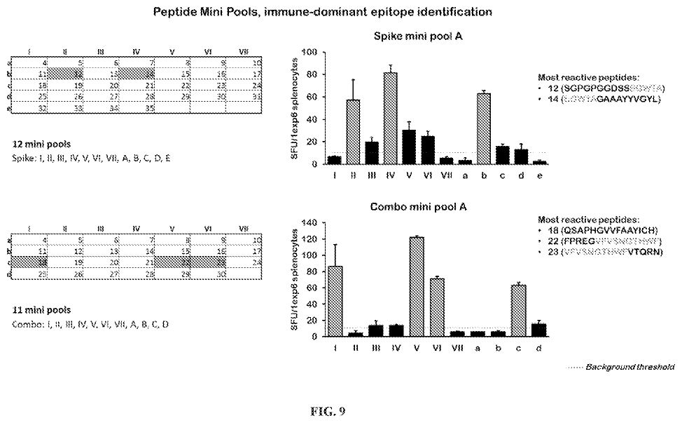

Vaccines induce potent CD8 T cell response as in FIG. 7, and CD4 responses observed in pool A in both spike and combined vaccine (FIG. 8). Vaccines were designed for presentation by human HLAs. Vaccinated mice only express one human HLA recognized by CD8 and no alleles recognized by CD4. No IFN-γ release observed in scrambled vaccine composed 33mers selected at random from SARS-CoV-2.

ELISPOT of expanded peptide mini-pools reveals overlapping sequences across 15mers (FIG. 9). Expanded minipool of pool A reveals reactive peptides contained on multiple 15mers (shaded sequences).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Alspach, E., Lussier, D. M., Miceli, A. P., Kizhvatov, I., DuPage, M., Luoma, A. M., . . . Schreiber, R. D. (2019). MHC-II neoantigens shape tumour immunity and response to immunotherapy. Nature, 574(7780), 696-701. doi:10.1038/s41586-019-1671-8
An, L.-L., Rodriguez, F., Harkins, S., Zhang, J., & Whitton, J. L. (2000). Quantitative and qualitative analyses of the immune responses induced by a multivalent minigene DNA vaccine. Vaccine, 18(20), 2132-2141. doi:https://doi.org/10.1016/S0264-410X(99)00546-0
Chen, J., Lee, K. H., Steinhauer, D. A., Stevens, D. J., Skehel, J. J., & Wiley, D. C. (1998). Structure of the Hemagglutinin Precursor Cleavage Site, a Determinant of Influenza Pathogenicity and the Origin of the Labile Conformation. Cell, 95(3), 409-417. doi:10.1016/S0092-8674(00)81771-7
Cheng, V. C. C., Lau, S. K. P., Woo, P. C. Y., & Yuen, K. Y. (2007). Severe Acute Respiratory Syndrome Coronavirus as an Agent of Emerging and Reemerging Infection. Clinical Microbiology Reviews, 20(4), 660-694. doi:10.1128/cmr.00023-07
Dejnirattisai, W., Supasa, P., Wongwiwat, W., Rouvinski, A., Barba-Spaeth, G., Duangchinda, T., . . . Screaton, G. R. (2016). Dengue virus sero-cross-reactivity drives antibody-dependent enhancement of infection with zika virus. Nature Immunology, 17(9), 1102-1108. doi:10.1038/ni.3515
Dowd, K. A., Ko, S.-Y., Morabito, K. M., Yang, E. S., Pelc, R. S., DeMaso, C. R., . . . Graham, B. S. (2016). Rapid development of a DNA vaccine for Zika virus. Science, 354(6309), 237-240. doi:10.1126/science.aai9137
Gagnon, S. J., Borbulevych, O. Y., Davis-Harrison, R. L., Baxter, T. K., Clemens, J. R., Armstrong, K. M., . . . Baker, B. M. (2005). Unraveling a Hotspot for TCR Recognition on HLA-A2: Evidence Against the Existence of Peptide-independent TCR Binding Determinants. Journal of Molecular Biology, 353(3), 556-573. doi:https://doi.org/10.1016/jmb.2005.08.024
Gragert, L., Madbouly, A., Freeman, J., & Maiers, M. (2013). Six-locus high resolution HLA haplotype frequencies derived from mixed-resolution DNA typing for the entire US donor registry. Hum Immunol, 74(10), 1313-1320. doi:10.1016/j.humimm.2013.06.025
Gras, S., Saulquin, X., Reiser, J.-B., Debeaupuis, E., Echasserieau, K., Kissenpfennig, A., . . . Housset, D. (2009). Structural Bases for the Affinity-Driven Selection of a Public TCR against a Dominant Human Cytomegalovirus Epitope. The Journal of Immunology, 183(1), 430-437. doi:10.4049/jimmuno1.0900556
Hiscox, J. A., Cavanagh, D., & Britton, P. (1995). Quantification of individual subgenomic mRNA species during replication of the coronavirus transmissible gastroenteritis virus. Virus Research, 36(2), 119-130. doi:https://doi.org/10.1016/0168-1702(94)00108-0
Irigoyen, N., Firth, A. E., Jones, J. D., Chung, B. Y. W., Siddell, S. G., & Brierley, I. (2016). High-Resolution Analysis of Coronavirus Gene Expression by RNA Sequencing and Ribosome Profiling. PLoS pathogens, 12(2), e1005473-e1005473. doi:10.1371/journal.ppat.1005473
Ishizuka, J., Stewart-Jones, G. B. E., van der Merwe, A., Bell, J. I., McMichael, A. J., & Jones, E. Y. (2008). The Structural Dynamics and Energetics of an Immunodominant T Cell Receptor Are Programmed by Its V&#x3b2; Domain. Immunity, 28(2), 171-182. doi:10.1016/j.immuni.2007.12.018
Jespersen, M. C., Peters, B., Nielsen, M., & Marcatili, P. (2017). BepiPred-2.0: improving sequence-based B-cell epitope prediction using conformational epitopes. Nucleic Acids Research, 45(W1), W24-W29. doi:10.1093/nar/gloc346
Krieg, A. M. (2008). Toll-like receptor 9 (TLR9) agonists in the treatment of cancer. Oncogene, 27(2), 161-167. doi:10.1038/sj.onc.1210911
Kringelum, J. V., Lundegaard, C., Lund, O., & Nielsen, M. (2012). Reliable B Cell Epitope Predictions: Impacts of Method Development and Improved Benchmarking. PLOS Computational Biology, 8(12), e1002829. doi:10.1371/journal.pcbi.1002829
Lu, Y.-C., Yao, X., Crystal, J. S., Li, Y. F., El-Gamil, M., Gross, C., . . . Robbins, P. F. (2014). Efficient identification of mutated cancer antigens recognized by T cells associated with durable tumor regressions. Clinical cancer research: an official journal of the American Association for Cancer Research, 20(13), 3401-3410. doi:10.1158/1078-0432.CCR-14-0433
Lurie, N., Saville, M., Hatchett, R., & Halton, J. (2020). Developing Covid-19 Vaccines at Pandemic Speed. New England Journal of Medicine. doi:10.1056/NEJMp2005630
McHeyzer-Williams, M., Okitsu, S., Wang, N., & McHeyzer-Williams, L. (2012). Molecular programming of B cell memory. Nature Reviews Immunology, 12(1), 24-34. doi:10.1038/nri3128
Pardi, N., Hogan, M. J., Pelc, R. S., Muramatsu, H., Andersen, H., DeMaso, C. R., . . . Weissman, D. (2017). Zika virus protection by a single low-dose nucleoside-modified mRNA vaccination. Nature, 543(7644), 248-251. doi:10.1038/nature21428
Richner, J. M., Himansu, S., Dowd, K. A., Butler, S. L., Salazar, V., Fox, J. M., . . . Diamond, M. S. (2017). Modified mRNA Vaccines Protect against Zika Virus Infection. Cell, 168(6), 1114-1125.e1110. doi:https://doi.org/10.1016/j.cell.2017.02.017
Shang, J., Ye, G., Shi, K., Wan, Y., Luo, C., Aihara, H., . . . Li, F. (2020). Structural basis of receptor recognition by SARS-CoV-2. Nature. doi:10.1038/s41586-020-2179-y
Sidney, J., Steen, A., Moore, C., Ngo, S., Chung, J., Peters, B., & Sette, A. (2010). Divergent motifs but overlapping binding repertoires of six HLA-DQ molecules frequently expressed in the worldwide human population. J Immunol, 185(7), 4189-4198. doi:10.4049/jimmunol.1001006
Sievers, F., Wilm, A., Dineen, D., Gibson, T. J., Karplus, K., Li, W., . . . Higgins, D. G. (2011). Fast, scalable generation of high-quality protein multiple sequence alignments using Clustal Omega. Mol Syst Biol, 7, 539. doi:10.1038/msb.2011.75
Solberg, O. D., Mack, S. J., Lancaster, A. K., Single, R. M., Tsai, Y., Sanchez-Mazas, A., & Thomson, G. (2008). Balancing selection and heterogeneity across the classical human leukocyte antigen loci: a meta-analytic review of 497 population studies. Hum Immunol, 69(7), 443-464. doi:10.1016/j.humimm.2008.05.001
Tetro, J. A. (2020). Is COVID-19 receiving ADE from other coronaviruses? Microbes and Infection, 22(2), 72-73. doi:https://doi.org/10.1016/j.micinf.2020.02.006
Thevarajan, I., Nguyen, T. H. O., Koutsakos, M., Druce, J., Caly, L., van de Sandt, C. E., . . . Kedzierska, K. (2020). Breadth of concomitant immune responses prior to patient recovery: a case report of non-severe COVID-19. Nature Medicine. doi:10.1038/s41591-020-0819-2
Walls, A. C., Park, Y.-J., Tortorici, M. A., Wall, A., McGuire, A. T., & Veesler, D. Structure, Function, and Antigenicity of the SARS-CoV-2 Spike Glycoprotein. Cell. doi:10.1016/j.cell.2020.02.058
Wan, Y., Shang, J., Sun, S., Tai, W., Chen, J., Geng, Q., . . . Li, F. (2020). Molecular Mechanism for Antibody-Dependent Enhancement of Coronavirus Entry. Journal of Virology, 94(5), e02015-02019. doi:10.1128/jvi.02015-19
Wang, Q., Zhang, L., Kuwahara, K., Li, L., Liu, Z., Li, T., . . . Liu, G. (2016). Immunodominant SARS Coronavirus Epitopes in Humans Elicited both Enhancing and Neutralizing Effects on Infection in Non-human Primates. ACS infectious diseases, 2(5), 361-376. doi:10.1021/acsinfecdis.6b00006
Wrapp, D., Wang, N., Corbett, K. S., Goldsmith, J. A., Hsieh, C.-L., Abiona, O., . . . McLellan, J. S. (2020). Cryo-EM structure of the 2019-nCoV spike in the prefusion conformation. Science, 367(6483), 1260-1263. doi:10.1126/science.abb2507
Xu, H., Zhong, L., Deng, J., Peng, J., Dan, H., Zeng, X., . . . Chen, Q. (2020). High expression of ACE2 receptor of 2019-nCoV on the epithelial cells of oral mucosa. International Journal of Oral Science, 12(1), 8. doi:10.1038/s41368-020-0074-x
Yarmarkovich, M., Farrel, A., Sison, A., di Marco, M., Raman, P., Parris, J. L., . . . Maris, J. M. (2020). Immunogenicity and Immune Silence in Human Cancer. Frontiers in Immunology, 11(69). doi:10.3389/fimmu.2020.00069
Zanetti, B. F., Ferreira, C. P., de Vasconcelos, J. R. C., & Han, S. W. (2019). scFv6.C4 DNA vaccine with fragment C of Tetanus toxin increases protective immunity against CEA-expressing tumor. Gene Therapy, 26(10), 441-454. doi:10.1038/s41434-019-0062-y
Zhang, H., Zhou, P., Wei, Y., Yue, H., Wang, Y., Hu, M., . . . Du, R. (2020). Histopathologic Changes and SARS-CoV-2 Immunostaining in the Lung of a Patient With COVID-19. Annals of Internal Medicine. doi:10.7326/m20-0533

1. A vaccine composition comprising one or more antigens selected from SEQ ID NOS: 1-65 and 82 or a nucleic acid encoding one or more antigens selected from SEQ ID NOS: 1-65 and 82.

2. The vaccine of claim 1, comprising two or more antigens selected from SEQ ID NOS: 1-65 and 82.

3. The vaccine of claim 1, comprising a fusion of two or more antigens selected from SEQ ID NOS: 1-65 and 82.

4. The vaccine of claim 3, comprising a linker between each epitope of the vaccine.

5. The vaccine of claim 4, wherein the linker is selected from GPGPG (SEQ ID NO: 79), AAY, HEYGAEALERAG (SEQ ID NO: 80), and EAAAK (SEQ ID NO: 81).

6. The vaccine of claim 4, wherein the order of epitopes and the linker used are chosen to prevent the formation of junctional epitopes having non-specific immunogenicity.

7. The vaccine of claim 1, comprising an ER signal peptide, a lysosome signal peptide, and/or a secretion signal peptide.

8. The vaccine of claim 1, further comprising a second open reading frame encoding SARS-CoV-2 spike protein.

9. The vaccine of claim 1, comprising a nucleic acid sequence according to nucleotides 850-2322 of SEQ ID NO: 66, nucleotides 850-2445 of SEQ ID NO: 69, or nucleotides 850-2772 of SEQ ID NO: 72.

10. The vaccine of claim 9, wherein the nucleic acid sequence is an RNA sequence corresponding to the recited DNA sequence.

11. The vaccine of claim 1, comprising a polypeptide encoded by nucleotides 850-2322 of SEQ ID NO: 66, nucleotides 850-2445 of SEQ ID NO: 69, or nucleotides 850-2772 of SEQ ID NO: 72.

12. The vaccine of claim 1, further comprising an adjuvant.

13. The vaccine of claim 1, further comprising a biological response modifier.

14. The vaccine of claim 1, further comprising a chemokine.

15. The vaccine of claim 1, wherein said one or more antigens are comprised in an intact dendritic cell.

16. The vaccine of claim 1, further comprising a TLR agonist.

17. The vaccine of claim 16, wherein the TLR agonist drives activation of signals 1 and 2 in antigen presenting cells.

18. The vaccine of claim 16, wherein the TLR agonist is tetanus toxoid.

19. The vaccine of claim 1, further comprising a SARS-CoV-2 B cell antigen or nucleic acid encoding a SARS-CoV-2 B cell antigen.

20. The vaccine of claim 1, wherein the nucleic acid is DNA or mRNA having an open reading frame encoding the one or more antigens.

21. The vaccine of claim 20, wherein the open reading frame is codon optimized.

22. A method of generating an anti-viral immune response is a subject comprising administering to the subject the vaccine composition of claim 1.

23. The method of claim 22, further comprising administering a second vaccine for SARS-CoV-2.


SEQUENCE LISTING (104 sequences)

SEQ ID NO: 1 - protein, 33 aa, Artificial Sequence
  Synthetic polypeptide
IAMSAFAMMFVKHKHAFLCLFLLPSLATVAYFN

SEQ ID NO: 2 - protein, 33 aa, Artificial Sequence
  Synthetic polypeptide
KVCEFQFCNDPFLGVYYHKNNKSWMESEFRVYS

SEQ ID NO: 3 - protein, 33 aa, Artificial Sequence
  Synthetic polypeptide
GDSSSGWTAGAAAYYVGYLQPRTFLLKYNENGT

SEQ ID NO: 4 - protein, 33 aa, Artificial Sequence
  Synthetic polypeptide
KPFERDISTEIYQAGSTPCNGVEGFNCYFPLQS

SEQ ID NO: 5 - protein, 33 aa, Artificial Sequence
  Synthetic polypeptide
YILFTRFFYVLGLAAIMQLFFSYFAVHFISNSW

SEQ ID NO: 6 - protein, 33 aa, Artificial Sequence
  Synthetic polypeptide
FAVHFISNSWLMWLIINLVQMAPISAMVRMYIF

SEQ ID NO: 7 - protein, 33 aa, Artificial Sequence
  Synthetic polypeptide
VTCLAYYFMRFRRAFGEYSHVVAFNTLLFLMSF

SEQ ID NO: 8 - protein, 33 aa, Artificial Sequence
  Synthetic polypeptide
LVGLMWLSYFIASFRLFARTRSMWSFNPETNIL

SEQ ID NO: 9 - protein, 33 aa, Artificial Sequence
  Synthetic polypeptide
LAHIQWMVMFTPLVPFWITIAYIICISTKHFYW

SEQ ID NO: 10 - protein, 33 aa, Artificial Sequence
  Synthetic polypeptide
TVVIGTSKFYGGWHNMLKTVYSDVENPHLMGWD

SEQ ID NO: 11 - protein, 33 aa, Artificial Sequence
  Synthetic polypeptide
FRLTLGVYDYLVSTQEFRYMNSQGLLPPKNSID

SEQ ID NO: 12 - protein, 33 aa, Artificial Sequence
  Synthetic polypeptide
YCFLGYFCTCYFGLFCLLNRYFRLTLGVYDYLV

SEQ ID NO: 13 - protein, 33 aa, Artificial Sequence
  Synthetic polypeptide
MMILSDDAVVCFNSTYASQGLVASIKNFKSVLY

SEQ ID NO: 14 - protein, 33 aa, Artificial Sequence
  Synthetic polypeptide
NFVRIIMRLWLCWKCRSKNPLLYDANYFLCWHT

SEQ ID NO: 15 - protein, 33 aa, Artificial Sequence
  Synthetic polypeptide
RLYLDAYNMMISAGFSLWVYKQFDTYNLWNTFT

SEQ ID NO: 16 - protein, 33 aa, Artificial Sequence
  Synthetic polypeptide
QQESPFVMMSAPPAQYELKHGTFTCASEYTGNY

SEQ ID NO: 17 - protein, 33 aa, Artificial Sequence
  Synthetic polypeptide
DVLLPLTQYNRYLALYNKYKYFSGAMDTTSYRE

SEQ ID NO: 18 - protein, 33 aa, Artificial Sequence
  Synthetic polypeptide
RSQMEIDFLELAMDEFIERYKLEGYAFEHIVYG

SEQ ID NO: 19 - protein, 33 aa, Artificial Sequence
  Synthetic polypeptide
DDTLRVEAFEYYHTTDPSFLGRYMSALNHTKKW

SEQ ID NO: 20 - protein, 33 aa, Artificial Sequence
  Synthetic polypeptide
ITSGWTFGAGAALQIPFAMQMAYRFNGIGVTQN

SEQ ID NO: 21 - protein, 33 aa, Artificial Sequence
  Synthetic polypeptide
SQLMCQPILLLDQALVSDVGDSAEVAVKMFDAY

SEQ ID NO: 22 - protein, 33 aa, Artificial Sequence
  Synthetic polypeptide
SLVLARKHTTCCSLSHRFYRLANECAQVLSEMV

SEQ ID NO: 23 - protein, 33 aa, Artificial Sequence
  Synthetic polypeptide
LVLARKHTTCCSLSHRFYRLANECAQVLSEMVM

SEQ ID NO: 24 - protein, 33 aa, Artificial Sequence
  Synthetic polypeptide
VLARKHTTCCSLSHRFYRLANECAQVLSEMVMC

SEQ ID NO: 25 - protein, 33 aa, Artificial Sequence
  Synthetic polypeptide
MTYRRLISMMGFKMNYQVNGYPNMFITREEAIR

SEQ ID NO: 26 - protein, 33 aa, Artificial Sequence
  Synthetic polypeptide
QVVDADSKIVQLSEISMDNSPNLAWPLIVTALR

SEQ ID NO: 27 - protein, 33 aa, Artificial Sequence
  Synthetic polypeptide
GVPVVDSYYSLLMPILTLTRALTAESHVDTDLT

SEQ ID NO: 28 - protein, 33 aa, Artificial Sequence
  Synthetic polypeptide
AYPLTKHPNQEYADVFHLYLQYIRKLHDELTGH

SEQ ID NO: 29 - protein, 33 aa, Artificial Sequence
  Synthetic polypeptide
GVLMSNLGMPSYCTGYREGYLNSTNVTIATYCT

SEQ ID NO: 30 - protein, 33 aa, Artificial Sequence
  Synthetic polypeptide
YFVKIGPERTCCLCDRRATCFSTASDTYACWHH

SEQ ID NO: 31 - protein, 33 aa, Artificial Sequence
  Synthetic polypeptide
FVKIGPERTCCLCDRRATCFSTASDTYACWHHS

SEQ ID NO: 32 - protein, 33 aa, Artificial Sequence
  Synthetic polypeptide
TRSTNSRIKASMPTTIAKNTVKSVGKFCLEASF

SEQ ID NO: 33 - protein, 33 aa, Artificial Sequence
  Synthetic polypeptide
IVVFDEISMATNYDLSVVNARLRAKHYVYIGDP

SEQ ID NO: 34 - protein, 33 aa, Artificial Sequence
  Synthetic polypeptide
KLLEQWNLVIGFLFLTWICLLQFAYANRNRFLY

SEQ ID NO: 35 - protein, 33 aa, Artificial Sequence
  Synthetic polypeptide
AYANRNRFLYIIKLIFLWLLWPVTLACFVLAAV

SEQ ID NO: 36 - protein, 33 aa, Artificial Sequence
  Synthetic polypeptide
TSRYWEPEFYEAMYTPHTVLQAVGACVLCNSQT

SEQ ID NO: 37 - protein, 33 aa, Artificial Sequence
  Synthetic polypeptide
KYEQYIKWPWYIWLGFIAGLIAIVMVTIMLCCM

SEQ ID NO: 38 - protein, 33 aa, Artificial Sequence
  Synthetic polypeptide
SMWALIISVTSNYSGVVTTVMFLARGIVFMCVE

SEQ ID NO: 39 - protein, 33 aa, Artificial Sequence
  Synthetic polypeptide
ETKFLTENLLLYIDINGNLHPDSATLVSDIDIT

SEQ ID NO: 40 - protein, 33 aa, Artificial Sequence
  Synthetic polypeptide
IAIILASFSASTSAFVETVKGLDYKAFKQIVES

SEQ ID NO: 41 - protein, 33 aa, Artificial Sequence
  Synthetic polypeptide
FASEAARVVRSIFSRTLETAQNSVRVLQKAAIT

SEQ ID NO: 42 - protein, 33 aa, Artificial Sequence
  Synthetic polypeptide
RTVYDDGARRVWTLMNVLTLVYKVYYGNALDQA

SEQ ID NO: 43 - protein, 33 aa, Artificial Sequence
  Synthetic polypeptide
MVLGSLAATVRLQAGNATEVPANSTVLSFCAFA

SEQ ID NO: 44 - protein, 33 aa, Artificial Sequence
  Synthetic polypeptide
NFTIKGSFLNGSCGSVGFNIDYDCVSFCYMHHM

SEQ ID NO: 45 - protein, 33 aa, Artificial Sequence
  Synthetic polypeptide
DFIDTKRGVYCCREHEHEIAWYTERSEKSYELQ

SEQ ID NO: 46 - protein, 33 aa, Artificial Sequence
  Synthetic polypeptide
ALRQMSCAAGTTQTACTDDNALAYYNTTKGGRF

SEQ ID NO: 47 - protein, 33 aa, Artificial Sequence
  Synthetic polypeptide
GEVFNATRFASVYAWNRKRISNCVADYSVLYNS

SEQ ID NO: 48 - protein, 33 aa, Artificial Sequence
  Synthetic polypeptide
TEHSWNADLYKLMGHFAWWTAFVTNVNASSSEA

SEQ ID NO: 49 - protein, 33 aa, Artificial Sequence
  Synthetic polypeptide
SYQTQTNSPRRARSVASQSIIAYTMSLGAENSV

SEQ ID NO: 50 - protein, 33 aa, Artificial Sequence
  Synthetic polypeptide
ATIPIQASLPFGWLIVGVALLAVFQSASKIITL

SEQ ID NO: 51 - protein, 33 aa, Artificial Sequence
  Synthetic polypeptide
CPDGVKHVYQLRARSVSPKLFIRQEEVQELYSP

SEQ ID NO: 52 - protein, 33 aa, Artificial Sequence
  Synthetic polypeptide
SEFSSLPSYAAFATAQEAYEQAVANGDSEVVLK

SEQ ID NO: 53 - protein, 33 aa, Artificial Sequence
  Synthetic polypeptide
NNLVVMAYITGGVVQLTSQWLTNIFGTVYEKLK

SEQ ID NO: 54 - protein, 33 aa, Artificial Sequence
  Synthetic polypeptide
HSYFTSDYYQLYSTQLSTDTGVEHVTFFIYNKI

SEQ ID NO: 55 - protein, 33 aa, Artificial Sequence
  Synthetic polypeptide
DQIGYYRRATRRIRGGDGKMKDLSPRWYFYYLG

SEQ ID NO: 56 - protein, 33 aa, Artificial Sequence
  Synthetic polypeptide
AQFAPSASAFFGMSRIGMEVTPSGTWLTYTGAI

SEQ ID NO: 57 - protein, 33 aa, Artificial Sequence
  Synthetic polypeptide
DLFLPFFSNVTWFHAIHVSGTNGTKRFDNPVLP

SEQ ID NO: 58 - protein, 33 aa, Artificial Sequence
  Synthetic polypeptide
NCTFEYVSQPFLMDLEGKQGNFKNLREFVFKNI

SEQ ID NO: 59 - protein, 33 aa, Artificial Sequence
  Synthetic polypeptide
NVYADSFVIRGDEVRQIAPGQTGKIADYNYKLP

SEQ ID NO: 60 - protein, 33 aa, Artificial Sequence
  Synthetic polypeptide
VGGNYNYLYRLFRKSNLKPFERDISTEIYQAGS

SEQ ID NO: 61 - protein, 33 aa, Artificial Sequence
  Synthetic polypeptide
FPLQSYGFQPTNGVGYQPYRVVVLSFELLHAPA

SEQ ID NO: 62 - protein, 33 aa, Artificial Sequence
  Synthetic polypeptide
QLNRALTGIAVEQDKNTQEVFAQVKQIYKTPPI

SEQ ID NO: 63 - protein, 33 aa, Artificial Sequence
  Synthetic polypeptide
LPDPSKPSKRSFIEDLLFNKVTLADAGFIKQYG

SEQ ID NO: 64 - protein, 33 aa, Artificial Sequence
  Synthetic polypeptide
SECVLGQSKRVDFCGKGYHLMSFPQSAPHGVVF

SEQ ID NO: 65 - protein, 33 aa, Artificial Sequence
  Synthetic polypeptide
ICHDGKAHFPREGVFVSNGTHWFVTQRNFYEPQ

SEQ ID NO: 66 - DNA, 4614 bp, Artificial Sequence
  Synthetic polynucleotide
  promoter (137)..(695): CMV promoter
  sig_peptide (724)..(780): secretion signal
  misc_feature (781)..(795): linker
  misc_feature (796)..(834): PADRE adjuvant
  misc_feature (835)..(849): linker
  misc_feature (850)..(948): epitope ORF1ab_5258 (SEQ ID NO 25)
  misc_feature (949)..(957): AAY linker
  misc_feature (958)..(1056): epitope ORF1ab_3619 (SEQ ID NO 1)
  misc_feature (1057)..(1071): GPGPG linker
  misc_feature (1072)..(1170): epitope ORF1ab_1624 (SEQ ID NO 16)
  misc_feature (1171)..(1185): GPGPG linker
  misc_feature (1186)..(1284): epitope N_82 (SEQ ID NO 56)
  misc_feature (1285)..(1299): GPGPG linker
  misc_feature (1300)..(1398): epitope N_305 (SEQ ID NO 60)
  misc_feature (1399)..(1413): GPGPG linker
  misc_feature (1414)..(1512): epitope ORF1ab_2562 (SEQ ID NO 18)
  misc_feature (1513)..(1527): GPGPG linker
  misc_feature (1528)..(1626): epitope M_87 (SEQ ID NO 5)
  misc_feature (1627)..(1641): GPGPG linker
  misc_feature (1642)..(1740): epitope ORF1ab_3804 (SEQ ID NO 8)
  misc_feature (1741)..(1755): GPGPG linker
  misc_feature (1756)..(1854): epitope M_38 (SEQ ID NO 32)
  misc_feature (1855)..(1869): GPGPG linker
  misc_feature (1870)..(1968): epitope ORF1ab_5304 (SEQ ID NO 33)
  misc_feature (1969)..(1983): GPGPG linker
  misc_feature (1984)..(2082): epitope M_15 (SEQ ID NO 31)
  misc_feature (2083)..(2097): GPGPG linker
  misc_feature (2098)..(2196): epitope ORF1ab_4622 (SEQ ID NO 24)
  misc_feature (2197)..(2223): HEYGAEALE linker
  misc_feature (2224)..(2322): epitope S_1205 (SEQ ID NO 34)
  misc_feature (2323)..(2337): GPGPG linker
  misc_feature (2338)..(2364): V5 tag
  polyA_site (2444)..(2668): BGH/pA
  misc_feature (2841)..(3635): Kanamicyn resistance
  misc_feature (3935)..(4608): pUC origin
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggagttcggactctcctgggtgtttctggtcgcgttgtttcgaggtgtgcaatgtgaagccgctgctaaggcaaagttcgtcgcagcctggacattgaaagcagcagccgaggcagccgcaaaggcttaccccctcacaaagcatcccaatcaagaatatgccgacgtgtttcacctatatcttcaatatattaggaagctgcatgatgaacttacagggcacgctgcgtatattgcgatgtccgcttttgctatgatgtttgtgaaacacaaacacgcttttctgtgcttgtttctcttgccaagcctggccactgtcgcatattttaatggcccggggcctggcgacgatacgctccgggtggaagctttcgaatattatcacaccactgatcccagttttctcggcagatacatgtcagccctcaatcatactaagaaatggggacccggccctggtgatcagatcggctattaccgccgggcaactcgtagaatccggggcggcgatggcaagatgaaggatcttagcccccgctggtacttctattatctgggtgggcccggtcccggcgcccaatttgcaccaagcgcctctgccttctttggaatgtcacggattggcatggaagtgaccccatctggtacatggctgacatacaccggagctattggtcctggaccgggttcccaacttatgtgccaacccattctgttgctggaccaggccctcgtgtctgatgtgggcgattccgcagaggtagctgtcaaaatgtttgacgcttatggtcccgggcctggattggtaggattgatgtggctgtcatatttcattgcttccttccgtttgttcgcaaggacaaggtctatgtggtctttcaatccggaaactaatatattgggcccaggtcccggttttcgactcacactcggcgtgtatgactaccttgtgtctactcaggagttcagatacatgaatagtcaaggtctgctcccgccaaagaatagtattgatggccctgggcccggagcatatgcgaacaggaatcgatttctttatattataaaattgatctttctttggctgctgtggcctgtgactttggcatgtttcgtactggcagccgtaggtcccggccccgggacgagcaggtattgggaacccgagttttatgaggccatgtatacaccccatactgtcctacaggccgttggtgcctgtgtgctctgtaactctcaaaccggtcccggccctggaaaactacttgagcaatggaacctggtgattgggttcctcttcctgacatggatttgcttgttgcagtttgcctatgcgaatagaaatagatttctctatgggccgggccccggcggagtgcccgtcgtagacagctattatagtctgctcatgccaatcttgacactaacacgagctctcaccgccgaatcacatgtcgacacagatttgacacacgaatatggcgcagaagccctcgagaaatatgaacaatatatcaaatggccctggtacatttggcttggtttcattgccggactcatcgcaatagttatggtcaccataatgctatgttgtatgggcccagggcccggaatccctaatcccttgttgggtcttgattgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 67 - DNA, 4623 bp, Artificial Sequence
  Synthetic polynucleotide
  promoter (137)..(695): CMV promoter
  sig_peptide (724)..(789): ER signal (ER AP (Ig K))
  misc_feature (790)..(804): linker
  misc_feature (805)..(843): PADRE adjuvant
  misc_feature (844)..(858): linker
  misc_feature (859)..(957): epitope ORF1ab_5258 (SEQ ID NO 25)
  misc_feature (958)..(966): AAY linker
  misc_feature (967)..(1065): epitope ORF1ab_3619 (SEQ ID NO 1)
  misc_feature (1066)..(1080): GPGPG linker
  misc_feature (1081)..(1179): epitope ORF1ab_1624 (SEQ ID NO 16)
  misc_feature (1180)..(1194): GPGPG linker
  misc_feature (1195)..(1293): epitope N_82 (SEQ ID NO 56)
  misc_feature (1294)..(1308): GPGPG linker
  misc_feature (1309)..(1407): epitope N_305 (SEQ ID NO 60)
  misc_feature (1408)..(1422): GPGPG linker
  misc_feature (1423)..(1521): epitope ORF1ab_2562 (SEQ ID NO 18)
  misc_feature (1522)..(1536): GPGPG linker
  misc_feature (1537)..(1635): epitope M_87 (SEQ ID NO 5)
  misc_feature (1636)..(1650): GPGPG linker
  misc_feature (1651)..(1749): epitope ORF1ab_3804 (SEQ ID NO 8)
  misc_feature (1750)..(1764): GPGPG linker
  misc_feature (1765)..(1863): epitope M_38 (SEQ ID NO 32)
  misc_feature (1864)..(1878): GPGPG linker
  misc_feature (1879)..(1977): epitope ORF1ab_5304 (SEQ ID NO 33)
  misc_feature (1978)..(1992): GPGPG linker
  misc_feature (1993)..(2091): epitope M_15 (SEQ ID NO 31)
  misc_feature (2092)..(2106): GPGPG linker
  misc_feature (2107)..(2205): epitope ORF1ab_4622 (SEQ ID NO 24)
  misc_feature (2206)..(2232): HEYGAEALE linker
  misc_feature (2233)..(2331): epitope S_1205 (SEQ ID NO 34)
  misc_feature (2332)..(2346): GPGPG linker
  misc_feature (2347)..(2373): V5 tag
  polyA_site (2453)..(2677): BGH/pA
  misc_feature (2850)..(3644): Kanamicyn resistance
  misc_feature (3944)..(4617): pUC origin
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgggaatgcaagtccagatacaaagtctgtttctacttctgctttgggtcccggggtcaaggggcgaagccgctgctaaggcaaagttcgtcgcagcctggacattgaaagcagcagccgaggcagccgcaaaggcttaccccctcacaaagcatcccaatcaagaatatgccgacgtgtttcacctatatcttcaatatattaggaagctgcatgatgaacttacagggcacgctgcgtatattgcgatgtccgcttttgctatgatgtttgtgaaacacaaacacgcttttctgtgcttgtttctcttgccaagcctggccactgtcgcatattttaatggcccggggcctggcgacgatacgctccgggtggaagctttcgaatattatcacaccactgatcccagttttctcggcagatacatgtcagccctcaatcatactaagaaatggggacccggccctggtgatcagatcggctattaccgccgggcaactcgtagaatccggggcggcgatggcaagatgaaggatcttagcccccgctggtacttctattatctgggtgggcccggtcccggcgcccaatttgcaccaagcgcctctgccttctttggaatgtcacggattggcatggaagtgaccccatctggtacatggctgacatacaccggagctattggtcctggaccgggttcccaacttatgtgccaacccattctgttgctggaccaggccctcgtgtctgatgtgggcgattccgcagaggtagctgtcaaaatgtttgacgcttatggtcccgggcctggattggtaggattgatgtggctgtcatatttcattgcttccttccgtttgttcgcaaggacaaggtctatgtggtctttcaatccggaaactaatatattgggcccaggtcccggttttcgactcacactcggcgtgtatgactaccttgtgtctactcaggagttcagatacatgaatagtcaaggtctgctcccgccaaagaatagtattgatggccctgggcccggagcatatgcgaacaggaatcgatttctttatattataaaattgatctttctttggctgctgtggcctgtgactttggcatgtttcgtactggcagccgtaggtcccggccccgggacgagcaggtattgggaacccgagttttatgaggccatgtatacaccccatactgtcctacaggccgttggtgcctgtgtgctctgtaactctcaaaccggtcccggccctggaaaactacttgagcaatggaacctggtgattgggttcctcttcctgacatggatttgcttgttgcagtttgcctatgcgaatagaaatagatttctctatgggccgggccccggcggagtgcccgtcgtagacagctattatagtctgctcatgccaatcttgacactaacacgagctctcaccgccgaatcacatgtcgacacagatttgacacacgaatatggcgcagaagccctcgagaaatatgaacaatatatcaaatggccctggtacatttggcttggtttcattgccggactcatcgcaatagttatggtcaccataatgctatgttgtatgggcccagggcccggaatccctaatcccttgttgggtcttgattgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 68 - DNA, 4629 bp, Artificial Sequence
  Synthetic polynucleotide
  promoter (137)..(695): CMV promoter
  sig_peptide (724)..(795): lysosomal signal (LAMP1 SP (m))
  misc_feature (796)..(810): linker
  misc_feature (811)..(849): PADRE adjuvant
  misc_feature (850)..(864): linker
  misc_feature (865)..(963): epitope ORF1ab_5258 (SEQ ID NO 25)
  misc_feature (964)..(972): AAY linker
  misc_feature (973)..(1071): epitope ORF1ab_3619 (SEQ ID NO 1)
  misc_feature (1072)..(1086): GPGPG linker
  misc_feature (1087)..(1185): epitope ORF1ab_1624 (SEQ ID NO 16)
  misc_feature (1186)..(1200): GPGPG linker
  misc_feature (1201)..(1299): epitope N_82 (SEQ ID NO 56)
  misc_feature (1300)..(1314): GPGPG linker
  misc_feature (1315)..(1413): epitope N_305 (SEQ ID NO 60)
  misc_feature (1414)..(1428): GPGPG linker
  misc_feature (1429)..(1527): epitope ORF1ab_2562 (SEQ ID NO 18)
  misc_feature (1528)..(1542): GPGPG linker
  misc_feature (1543)..(1641): epitope M_87 (SEQ ID NO 5)
  misc_feature (1642)..(1656): GPGPG linker
  misc_feature (1657)..(1755): epitope ORF1ab_3804 (SEQ ID NO 8)
  misc_feature (1756)..(1770): GPGPG linker
  misc_feature (1771)..(1869): epitope M_38 (SEQ ID NO 32)
  misc_feature (1870)..(1884): GPGPG linker
  misc_feature (1885)..(1983): epitope ORF1ab_5304 (SEQ ID NO 33)
  misc_feature (1984)..(1998): GPGPG linker
  misc_feature (1999)..(2097): epitope M_15 (SEQ ID NO 31)
  misc_feature (2098)..(2112): GPGPG linker
  misc_feature (2113)..(2211): epitope ORF1ab_4622 (SEQ ID NO 24)
  misc_feature (2212)..(2238): HEYGAEALE linker
  misc_feature (2239)..(2337): epitope S_1205 (SEQ ID NO 34)
  misc_feature (2338)..(2352): GPGPG linker
  misc_feature (2353)..(2379): V5 tag
  polyA_site (2459)..(2683): BGH/pA
  misc_feature (2856)..(3650): Kanamicyn resistance
  misc_feature (3950)..(4623): pUC origin
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggcagccccaggtgcacgacgacccctgctactcctactactcgcaggcctggcacacggtgcaagtgctgaagccgctgctaaggcaaagttcgtcgcagcctggacattgaaagcagcagccgaggcagccgcaaaggcttaccccctcacaaagcatcccaatcaagaatatgccgacgtgtttcacctatatcttcaatatattaggaagctgcatgatgaacttacagggcacgctgcgtatattgcgatgtccgcttttgctatgatgtttgtgaaacacaaacacgcttttctgtgcttgtttctcttgccaagcctggccactgtcgcatattttaatggcccggggcctggcgacgatacgctccgggtggaagctttcgaatattatcacaccactgatcccagttttctcggcagatacatgtcagccctcaatcatactaagaaatggggacccggccctggtgatcagatcggctattaccgccgggcaactcgtagaatccggggcggcgatggcaagatgaaggatcttagcccccgctggtacttctattatctgggtgggcccggtcccggcgcccaatttgcaccaagcgcctctgccttctttggaatgtcacggattggcatggaagtgaccccatctggtacatggctgacatacaccggagctattggtcctggaccgggttcccaacttatgtgccaacccattctgttgctggaccaggccctcgtgtctgatgtgggcgattccgcagaggtagctgtcaaaatgtttgacgcttatggtcccgggcctggattggtaggattgatgtggctgtcatatttcattgcttccttccgtttgttcgcaaggacaaggtctatgtggtctttcaatccggaaactaatatattgggcccaggtcccggttttcgactcacactcggcgtgtatgactaccttgtgtctactcaggagttcagatacatgaatagtcaaggtctgctcccgccaaagaatagtattgatggccctgggcccggagcatatgcgaacaggaatcgatttctttatattataaaattgatctttctttggctgctgtggcctgtgactttggcatgtttcgtactggcagccgtaggtcccggccccgggacgagcaggtattgggaacccgagttttatgaggccatgtatacaccccatactgtcctacaggccgttggtgcctgtgtgctctgtaactctcaaaccggtcccggccctggaaaactacttgagcaatggaacctggtgattgggttcctcttcctgacatggatttgcttgttgcagtttgcctatgcgaatagaaatagatttctctatgggccgggccccggcggagtgcccgtcgtagacagctattatagtctgctcatgccaatcttgacactaacacgagctctcaccgccgaatcacatgtcgacacagatttgacacacgaatatggcgcagaagccctcgagaaatatgaacaatatatcaaatggccctggtacatttggcttggtttcattgccggactcatcgcaatagttatggtcaccataatgctatgttgtatgggcccagggcccggaatccctaatcccttgttgggtcttgattgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 69 - DNA, 4737 bp, Artificial Sequence
  Synthetic polynucleotide
  promoter (137)..(695): CMV promoter
  sig_peptide (724)..(780): secretion signal
  misc_feature (781)..(795): linker
  misc_feature (796)..(834): PADRE adjuvant
  misc_feature (835)..(849): linker
  misc_feature (850)..(948): epitope S_339 (SEQ ID NO 45)
  misc_feature (949)..(963): GPGPG linker
  misc_feature (964)..(1062): epitope S_1030 (SEQ ID NO 55)
  misc_feature (1063)..(1071): AAY linker
  misc_feature (1072)..(1170): epitope S_1081 (SEQ ID NO 59)
  misc_feature (1171)..(1185): GPGPG linker
  misc_feature (1186)..(1284): epitope S_252 (SEQ ID NO 41)
  misc_feature (1285)..(1299): GPGPG linker
  misc_feature (1300)..(1398): epitope S_394 (SEQ ID NO 63)
  misc_feature (1399)..(1413): GPGPG linker
  misc_feature (1414)..(1512): epitope S_490 (SEQ ID NO 61)
  misc_feature (1513)..(1527): GPGPG linker
  misc_feature (1528)..(1626): epitope S_673 (SEQ ID NO 47)
  misc_feature (1627)..(1662): HEYGAEALERAG linker
  misc_feature (1663)..(1761): epitope S_462 (SEQ ID NO 64)
  misc_feature (1762)..(1776): GPGPG linker
  misc_feature (1777)..(1875): epitope S_806 (SEQ ID NO 62)
  misc_feature (1876)..(1890): GPGPG linker
  misc_feature (1891)..(1989): epitope S_445 (SEQ ID NO 65)
  misc_feature (1990)..(2004): GPGPG linker
  misc_feature (2005)..(2103): epitope S_882 (SEQ ID NO 17)
  misc_feature (2104)..(2118): GPGPG linker
  misc_feature (2119)..(2217): epitope S_1205 (SEQ ID NO 34)
  misc_feature (2218)..(2232): GPGPG linker
  misc_feature (2233)..(2331): epitope S_603 (SEQ ID NO 104)
  misc_feature (2332)..(2346): GPGPG linker
  misc_feature (2347)..(2445): epitope S_165 (SEQ ID NO 58)
  misc_feature (2446)..(2460): GPGPG linker
  misc_feature (2461)..(2487): V5 tag
  polyA_site (2567)..(2791): BGH/pA
  misc_feature (2964)..(3758): Kanamicyn resistance
  misc_feature (4058)..(4731): pUC origin
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggagttcggactctcctgggtgtttctggtcgcgttgtttcgaggtgtgcaatgtgaagccgctgctaaggcaaagttcgtcgcagcctggacattgaaagcagcagccgaggcagccgcaaagggggaagtctttaatgcaacccgctttgcttcagtatatgcgtggaatagaaaacgcatcagtaactgcgtcgcagattatagtgttctctacaactccggacctgggcctgggagtgaatgtgtcctaggccaaagtaaacgtgttgacttctgcggaaaaggatatcacttgatgtcatttcctcagtccgcaccacatggcgttgtctttgcagcgtacatttgccacgatgggaaagcacacttcccaagagagggtgtgtttgttagtaacggcacgcactggttcgtgacccaaagaaacttttacgaaccgcagggtccagggccgggtggagactctagttccggctggactgccggagccgcggcgtattacgtcgggtacttgcaacccaggacatttctgcttaagtataacgaaaatggaacagggccaggacctgggaatgtctatgcggatagttttgtcattcggggcgatgaagtccgccaaatcgcaccgggtcaaaccggcaagatcgctgactacaattacaaactcccagggcccggtccaggttttccgctacaatcttacggctttcaaccaacgaacggggtcggttaccagccttatagggtcgtcgtactgtcttttgaacttctccatgcacccgccggtccaggccccgggagctaccagacacaaaccaactctcctcgccgggcacgcagcgtagcaagccaatcaataattgcttacacaatgtcactgggtgctgaaaattcagtgcatgagtacggagcagaagctctggagcgggccggcaaaccgtttgagagggatattagtaccgagatataccaagcagggtccactccgtgtaacggggtggaaggctttaattgctatttcccacttcaatcagggcctggccccggtttgcctgaccctagtaagcccagtaaaaggtcctttatcgaagatctcctgtttaataaagtgaccctcgctgacgctgggttcatcaagcagtacggcggccctggtcctggagtcggcggaaactataattacctatatagactcttccgcaaatctaacttgaagccattcgaacgcgatatatctacagaaatctatcaagctggttccgggcccggaccaggtatcacaagcggttggactttcggcgctggagccgcattgcagatccctttcgccatgcagatggcttacagattcaatggaataggagtgacgcaaaatggtccgggaccaggtaagtacgaacaatatataaagtggccatggtatatctggctggggtttatcgccggcttgatcgccatagttatggttacaatcatgctctgttgcatggggcccggtcccggaaatactagcaatcaagtcgcggtcctctaccagggagtgaattgtacggaagttccggtggctatccacgcggatcagctcacgcccacatggcgggtaggaccagggccaggcaactgcacctttgagtatgtctctcagcccttccttatggatctggaaggaaaacaaggaaattttaagaatctccgagagttcgtatttaagaatattggcccagggcccggaatccctaatcccttgttgggtcttgattgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 70 - DNA, 4746 bp, Artificial Sequence
  Synthetic polynucleotide
  promoter (137)..(695): CMV promoter
  sig_peptide (724)..(789): ER signal (ER AP (Ig K))
  misc_feature (790)..(804): linker
  misc_feature (805)..(843): PADRE adjuvant
  misc_feature (844)..(858): linker
  misc_feature (859)..(957): epitope S_339 (SEQ ID NO 45)
  misc_feature (958)..(972): GPGPG linker
  misc_feature (973)..(1071): epitope S_1030 (SEQ ID NO 55)
  misc_feature (1072)..(1080): AAY linker
  misc_feature (1081)..(1179): epitope S_1081 (SEQ ID NO 59)
  misc_feature (1180)..(1194): GPGPG linker
  misc_feature (1195)..(1293): epitope S_252 (SEQ ID NO 41)
  misc_feature (1294)..(1308): GPGPG linker
  misc_feature (1309)..(1407): epitope S_394 (SEQ ID NO 63)
  misc_feature (1408)..(1422): GPGPG linker
  misc_feature (1423)..(1521): epitope S_490 (SEQ ID NO 61)
  misc_feature (1522)..(1536): GPGPG linker
  misc_feature (1537)..(1635): epitope S_673 (SEQ ID NO 47)
  misc_feature (1636)..(1671): HEYGAEALERAG linker
  misc_feature (1672)..(1770): epitope S_462 (SEQ ID NO 64)
  misc_feature (1771)..(1785): GPGPG linker
  misc_feature (1786)..(1884): epitope S_806 (SEQ ID NO 62)
  misc_feature (1885)..(1899): GPGPG linker
  misc_feature (1900)..(1998): epitope S_445 (SEQ ID NO 65)
  misc_feature (1999)..(2013): GPGPG linker
  misc_feature (2014)..(2112): epitope S_882 (SEQ ID NO 17)
  misc_feature (2113)..(2127): GPGPG linker
  misc_feature (2128)..(2226): epitope S_1205 (SEQ ID NO 34)
  misc_feature (2227)..(2241): GPGPG linker
  misc_feature (2242)..(2340): epitope S_603 (SEQ ID NO 104)
  misc_feature (2341)..(2355): GPGPG linker
  misc_feature (2356)..(2454): epitope S_165 (SEQ ID NO 58)
  misc_feature (2455)..(2469): GPGPG linker
  misc_feature (2470)..(2496): V5 tag
  polyA_site (2576)..(2800): BGH/pA
  misc_feature (2973)..(3767): Kanamicyn resistance
  misc_feature (4067)..(4740): pUC origin
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgggaatgcaagtccagatacaaagtctgtttctacttctgctttgggtcccggggtcaaggggcgaagccgctgctaaggcaaagttcgtcgcagcctggacattgaaagcagcagccgaggcagccgcaaagggggaagtctttaatgcaacccgctttgcttcagtatatgcgtggaatagaaaacgcatcagtaactgcgtcgcagattatagtgttctctacaactccggacctgggcctgggagtgaatgtgtcctaggccaaagtaaacgtgttgacttctgcggaaaaggatatcacttgatgtcatttcctcagtccgcaccacatggcgttgtctttgcagcgtacatttgccacgatgggaaagcacacttcccaagagagggtgtgtttgttagtaacggcacgcactggttcgtgacccaaagaaacttttacgaaccgcagggtccagggccgggtggagactctagttccggctggactgccggagccgcggcgtattacgtcgggtacttgcaacccaggacatttctgcttaagtataacgaaaatggaacagggccaggacctgggaatgtctatgcggatagttttgtcattcggggcgatgaagtccgccaaatcgcaccgggtcaaaccggcaagatcgctgactacaattacaaactcccagggcccggtccaggttttccgctacaatcttacggctttcaaccaacgaacggggtcggttaccagccttatagggtcgtcgtactgtcttttgaacttctccatgcacccgccggtccaggccccgggagctaccagacacaaaccaactctcctcgccgggcacgcagcgtagcaagccaatcaataattgcttacacaatgtcactgggtgctgaaaattcagtgcatgagtacggagcagaagctctggagcgggccggcaaaccgtttgagagggatattagtaccgagatataccaagcagggtccactccgtgtaacggggtggaaggctttaattgctatttcccacttcaatcagggcctggccccggtttgcctgaccctagtaagcccagtaaaaggtcctttatcgaagatctcctgtttaataaagtgaccctcgctgacgctgggttcatcaagcagtacggcggccctggtcctggagtcggcggaaactataattacctatatagactcttccgcaaatctaacttgaagccattcgaacgcgatatatctacagaaatctatcaagctggttccgggcccggaccaggtatcacaagcggttggactttcggcgctggagccgcattgcagatccctttcgccatgcagatggcttacagattcaatggaataggagtgacgcaaaatggtccgggaccaggtaagtacgaacaatatataaagtggccatggtatatctggctggggtttatcgccggcttgatcgccatagttatggttacaatcatgctctgttgcatggggcccggtcccggaaatactagcaatcaagtcgcggtcctctaccagggagtgaattgtacggaagttccggtggctatccacgcggatcagctcacgcccacatggcgggtaggaccagggccaggcaactgcacctttgagtatgtctctcagcccttccttatggatctggaaggaaaacaaggaaattttaagaatctccgagagttcgtatttaagaatattggcccagggcccggaatccctaatcccttgttgggtcttgattgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 71 - DNA, 4752 bp, Artificial Sequence
  Synthetic polynucleotide
  promoter (137)..(695): CMV promoter
  sig_peptide (724)..(795): lysosomal signal (LAMP1 SP (m))
  misc_feature (796)..(810): linker
  misc_feature (811)..(849): PADRE adjuvant
  misc_feature (850)..(864): linker
  misc_feature (865)..(963): epitope S_339 (SEQ ID NO 45)
  misc_feature (964)..(978): GPGPG linker
  misc_feature (979)..(1077): epitope S_1030 (SEQ ID NO 55)
  misc_feature (1078)..(1086): AAY linker
  misc_feature (1087)..(1185): epitope S_1081 (SEQ ID NO 59)
  misc_feature (1186)..(1200): GPGPG linker
  misc_feature (1201)..(1299): epitope S_252 (SEQ ID NO 41)
  misc_feature (1300)..(1314): GPGPG linker
  misc_feature (1315)..(1413): epitope S_394 (SEQ ID NO 63)
  misc_feature (1414)..(1428): GPGPG linker
  misc_feature (1429)..(1527): epitope S_490 (SEQ ID NO 61)
  misc_feature (1528)..(1542): GPGPG linker
  misc_feature (1543)..(1641): epitope S_673 (SEQ ID NO 47)
  misc_feature (1642)..(1677): HEYGAEALERAG linker
  misc_feature (1678)..(1776): epitope S_462 (SEQ ID NO 64)
  misc_feature (1777)..(1791): GPGPG linker
  misc_feature (1792)..(1890): epitope S_806 (SEQ ID NO 62)
  misc_feature (1891)..(1905): GPGPG linker
  misc_feature (1906)..(2004): epitope S_445 (SEQ ID NO 65)
  misc_feature (2005)..(2019): GPGPG linker
  misc_feature (2020)..(2118): epitope S_882 (SEQ ID NO 17)
  misc_feature (2119)..(2133): GPGPG linker
  misc_feature (2134)..(2232): epitope S_1205 (SEQ ID NO 34)
  misc_feature (2233)..(2247): GPGPG linker
  misc_feature (2248)..(2346): epitope S_603 (SEQ ID NO 104)
  misc_feature (2347)..(2361): GPGPG linker
  misc_feature (2362)..(2460): epitope S_165 (SEQ ID NO 58)
  misc_feature (2461)..(2475): GPGPG linker
  misc_feature (2476)..(2502): V5 tag
  polyA_site (2582)..(2806): BGH/pA
  misc_feature (2979)..(3773): Kanamicyn resistance
  misc_feature (4073)..(4746): pUC origin
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggcagccccaggtgcacgacgacccctgctactcctactactcgcaggcctggcacacggtgcaagtgctgaagccgctgctaaggcaaagttcgtcgcagcctggacattgaaagcagcagccgaggcagccgcaaagggggaagtctttaatgcaacccgctttgcttcagtatatgcgtggaatagaaaacgcatcagtaactgcgtcgcagattatagtgttctctacaactccggacctgggcctgggagtgaatgtgtcctaggccaaagtaaacgtgttgacttctgcggaaaaggatatcacttgatgtcatttcctcagtccgcaccacatggcgttgtctttgcagcgtacatttgccacgatgggaaagcacacttcccaagagagggtgtgtttgttagtaacggcacgcactggttcgtgacccaaagaaacttttacgaaccgcagggtccagggccgggtggagactctagttccggctggactgccggagccgcggcgtattacgtcgggtacttgcaacccaggacatttctgcttaagtataacgaaaatggaacagggccaggacctgggaatgtctatgcggatagttttgtcattcggggcgatgaagtccgccaaatcgcaccgggtcaaaccggcaagatcgctgactacaattacaaactcccagggcccggtccaggttttccgctacaatcttacggctttcaaccaacgaacggggtcggttaccagccttatagggtcgtcgtactgtcttttgaacttctccatgcacccgccggtccaggccccgggagctaccagacacaaaccaactctcctcgccgggcacgcagcgtagcaagccaatcaataattgcttacacaatgtcactgggtgctgaaaattcagtgcatgagtacggagcagaagctctggagcgggccggcaaaccgtttgagagggatattagtaccgagatataccaagcagggtccactccgtgtaacggggtggaaggctttaattgctatttcccacttcaatcagggcctggccccggtttgcctgaccctagtaagcccagtaaaaggtcctttatcgaagatctcctgtttaataaagtgaccctcgctgacgctgggttcatcaagcagtacggcggccctggtcctggagtcggcggaaactataattacctatatagactcttccgcaaatctaacttgaagccattcgaacgcgatatatctacagaaatctatcaagctggttccgggcccggaccaggtatcacaagcggttggactttcggcgctggagccgcattgcagatccctttcgccatgcagatggcttacagattcaatggaataggagtgacgcaaaatggtccgggaccaggtaagtacgaacaatatataaagtggccatggtatatctggctggggtttatcgccggcttgatcgccatagttatggttacaatcatgctctgttgcatggggcccggtcccggaaatactagcaatcaagtcgcggtcctctaccagggagtgaattgtacggaagttccggtggctatccacgcggatcagctcacgcccacatggcgggtaggaccagggccaggcaactgcacctttgagtatgtctctcagcccttccttatggatctggaaggaaaacaaggaaattttaagaatctccgagagttcgtatttaagaatattggcccagggcccggaatccctaatcccttgttgggtcttgattgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 72 - DNA, 5064 bp, Artificial Sequence
  Synthetic polynucleotide
  promoter (137)..(695): CMV promoter
  sig_peptide (724)..(780): secretion signal
  misc_feature (781)..(795): linker
  misc_feature (796)..(834): PADRE adjuvant
  misc_feature (835)..(849): linker
  misc_feature (850)..(948): epitope S_339 (SEQ ID NO 45)
  misc_feature (949)..(963): GPGPG linker
  misc_feature (964)..(1062): epitope S_252 (SEQ ID NO 41)
  misc_feature (1063)..(1077): GPGPG linker
  misc_feature (1078)..(1176): epitope S_806 (SEQ ID NO 62)
  misc_feature (1177)..(1191): GPGPG linker
  misc_feature (1192)..(1290): epitope S_462 (SEQ ID NO 64)
  misc_feature (1291)..(1305): GPGPG linker
  misc_feature (1306)..(1404): epitope N_305 (SEQ ID NO 60)
  misc_feature (1405)..(1419): GPGPG linker
  misc_feature (1420)..(1518): epitope S_1081 (SEQ ID NO 59)
  misc_feature (1519)..(1533): GPGPG linker
  misc_feature (1534)..(1632): epitope S_673 (SEQ ID NO 47)
  misc_feature (1633)..(1647): GPGPG linker
  misc_feature (1648)..(1746): epitope S_603 (SEQ ID NO 104)
  misc_feature (1747)..(1761): GPGPG linker
  misc_feature (1762)..(1860): epitope ORF1ab_5304 (SEQ ID NO 33)
  misc_feature (1861)..(1875): GPGPG linker
  misc_feature (1876)..(1974): epitope S_882 (SEQ ID NO 17)
  misc_feature (1975)..(1989): GPGPG linker
  misc_feature (1990)..(2088): epitope M_38 (SEQ ID NO 32)
  misc_feature (2089)..(2103): GPGPG linker
  misc_feature (2104)..(2202): epitope M_15 (SEQ ID NO 31)
  misc_feature (2203)..(2217): GPGPG linker
  misc_feature (2218)..(2316): epitope ORF1ab_4622 (SEQ ID NO 24)
  misc_feature (2317)..(2331): GPGPG linker
  misc_feature (2332)..(2430): epitope ORF1ab_3804 (SEQ ID NO 8)
  misc_feature (2431)..(2445): GPGPG linker
  misc_feature (2446)..(2544): epitope S_1205 (SEQ ID NO 34)
  misc_feature (2545)..(2559): GPGPG linker
  misc_feature (2560)..(2658): epitope M_87 (SEQ ID NO 5)
  misc_feature (2659)..(2673): GPGPG linker
  misc_feature (2674)..(2772): epitope ORF1ab_3619 (SEQ ID NO 1)
  misc_feature (2773)..(2787): GPGPG linker
  misc_feature (2788)..(2814): V5 tag
  polyA_site (2894)..(3118): BGH/pA
  misc_feature (3291)..(4085): Kanamicyn resistance
  misc_feature (4385)..(5058): pUC origin
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggagttcggactctcctgggtgtttctggtcgcgttgtttcgaggtgtgcaatgtgaagccgctgctaaggcaaagttcgtcgcagcctggacattgaaagcagcagccgaggcagccgcaaagggcgaggtcttcaacgcaactaggtttgcttcagtctatgcttggaaccgcaagcggatctctaactgcgtggctgactatagtgtcttgtataatagtggaccaggtcccggtggagactcatccagcgggtggaccgctggagcagccgcttattatgtgggttatctccaacctcgcactttcttgctcaagtacaatgaaaacggaacaggacctggacccggtcttcctgacccttctaagccatccaagagatcctttatagaagacttgctgttcaacaaggttactcttgctgatgcagggtttatcaaacaatacggcggacctggcccaggaaagccctttgaaagggatatcagtactgagatttaccaagctgggtctaccccttgtaatggtgttgaagggtttaattgctacttccctctccagagtggtcctgggcctggggcccaattcgctccaagcgccagtgccttcttcggaatgtctaggatcggtatggaagtcactcctagtgggacatggcttacctacactggggccattggcccaggcccaggtatctgccacgatggtaaagcccattttccccgtgaaggcgtctttgtttctaatgggacacattggtttgtgactcaacgtaacttttatgaaccacaaggacctggtcccggcagctatcaaacccaaaccaatagcccccgccgtgcgcgtagtgttgcctctcaatccattatagcatacaccatgtctttgggcgccgagaatagcgtgggccccggaccaggcaatactagtaatcaggtcgccgtactgtatcaaggtgtgaattgtaccgaggtccccgtcgcaatccacgcagatcaattgactccaacatggcgggttggaccgggacctggaacatctcgctattgggaacctgaattttacgaagcgatgtacacaccccacacagtcttgcaggccgttggagcttgtgttctctgtaattcccaaacaggaccggggcccggtattacaagtgggtggacctttggagctggcgcagcactccagatacctttcgcaatgcaaatggcataccggtttaatggaataggggtaacgcagaacggtccgggaccgggcgcgtatgcgaacagaaatagatttctctatataattaaactaatcttcctgtggctcctctggccagtcaccttggcctgcttcgtgcttgccgccgtcggccctgggcccgggaaattgcttgagcaatggaacctcgttatcgggttcttgttcctgacgtggatttgtctactgcaattcgcttatgctaaccgcaatagattcctttatggccccggacctggcggggtccctgttgtggactcctattattctctcctgatgcccatactcactttgacacgggccctcactgccgaaagtcacgtggacacagatctaacaggccccggccccggttttcgcctgactctcggggtctacgattatctcgtgagcacccaagagtttcgctatatgaactctcaagggctgcttccgcccaagaactcaatcgatggaccaggtccgggcaaatatgagcagtacattaagtggccctggtacatttggttgggatttatcgctggcctcattgcaattgtcatggttactatcatgctgtgttgtatgggcccaggtccaggactcgtcggactcatgtggctgtcctacttcatcgcgtcttttagactctttgcaaggacacgttccatgtggagttttaaccctgaaactaatattctgggtcctggtcccgggatagctatgagcgcctttgccatgatgtttgtcaaacataaacatgcttttctttgcttgttcctcttgcctagtctagcaaccgtggcctacttcaatggcccagggcccggaatccctaatcccttgttgggtcttgattgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 73 - DNA, 5073 bp, Artificial Sequence
  Synthetic polynucleotide
  promoter (137)..(695): CMV promoter
  sig_peptide (724)..(789): ER signal (ER AP (Ig K))
  misc_feature (790)..(804): linker
  misc_feature (805)..(843): PADRE adjuvant
  misc_feature (844)..(858): linker
  misc_feature (859)..(957): epitope S_339 (SEQ ID NO 45)
  misc_feature (958)..(972): GPGPG linker
  misc_feature (973)..(1071): epitope S_252 (SEQ ID NO 41)
  misc_feature (1072)..(1086): GPGPG linker
  misc_feature (1087)..(1185): epitope S_806 (SEQ ID NO 62)
  misc_feature (1186)..(1200): GPGPG linker
  misc_feature (1201)..(1299): epitope S_462 (SEQ ID NO 64)
  misc_feature (1300)..(1314): GPGPG linker
  misc_feature (1315)..(1413): epitope N_305 (SEQ ID NO 60)
  misc_feature (1414)..(1428): GPGPG linker
  misc_feature (1429)..(1527): epitope S_1081 (SEQ ID NO 59)
  misc_feature (1528)..(1542): GPGPG linker
  misc_feature (1543)..(1641): epitope S_673 (SEQ ID NO 47)
  misc_feature (1642)..(1656): GPGPG linker
  misc_feature (1657)..(1755): epitope S_603 (SEQ ID NO 104)
  misc_feature (1756)..(1770): GPGPG linker
  misc_feature (1771)..(1869): epitope ORF1ab_5304 (SEQ ID NO 33)
  misc_feature (1870)..(1884): GPGPG linker
  misc_feature (1885)..(1983): epitope S_882 (SEQ ID NO 17)
  misc_feature (1984)..(1998): GPGPG linker
  misc_feature (1999)..(2097): epitope M_38 (SEQ ID NO 32)
  misc_feature (2098)..(2112): GPGPG linker
  misc_feature (2113)..(2211): epitope M_15 (SEQ ID NO 31)
  misc_feature (2212)..(2226): GPGPG linker
  misc_feature (2227)..(2325): epitope ORF1ab_4622 (SEQ ID NO 24)
  misc_feature (2326)..(2340): GPGPG linker
  misc_feature (2341)..(2439): epitope ORF1ab_3804 (SEQ ID NO 8)
  misc_feature (2440)..(2454): GPGPG linker
  misc_feature (2455)..(2553): epitope S_1205 (SEQ ID NO 34)
  misc_feature (2554)..(2568): GPGPG linker
  misc_feature (2569)..(2667): epitope M_87 (SEQ ID NO 5)
  misc_feature (2668)..(2682): GPGPG linker
  misc_feature (2683)..(2781): epitope ORF1ab_3619 (SEQ ID NO 1)
  misc_feature (2782)..(2796): GPGPG linker
  misc_feature (2797)..(2823): V5 tag
  polyA_site (2903)..(3127): BGH/pA
  misc_feature (3300)..(4094): Kanamicyn resistance
  misc_feature (4394)..(5067): pUC origin
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgggaatgcaagtccagatacaaagtctgtttctacttctgctttgggtcccggggtcaaggggcgaagccgctgctaaggcaaagttcgtcgcagcctggacattgaaagcagcagccgaggcagccgcaaagggcgaggtcttcaacgcaactaggtttgcttcagtctatgcttggaaccgcaagcggatctctaactgcgtggctgactatagtgtcttgtataatagtggaccaggtcccggtggagactcatccagcgggtggaccgctggagcagccgcttattatgtgggttatctccaacctcgcactttcttgctcaagtacaatgaaaacggaacaggacctggacccggtcttcctgacccttctaagccatccaagagatcctttatagaagacttgctgttcaacaaggttactcttgctgatgcagggtttatcaaacaatacggcggacctggcccaggaaagccctttgaaagggatatcagtactgagatttaccaagctgggtctaccccttgtaatggtgttgaagggtttaattgctacttccctctccagagtggtcctgggcctggggcccaattcgctccaagcgccagtgccttcttcggaatgtctaggatcggtatggaagtcactcctagtgggacatggcttacctacactggggccattggcccaggcccaggtatctgccacgatggtaaagcccattttccccgtgaaggcgtctttgtttctaatgggacacattggtttgtgactcaacgtaacttttatgaaccacaaggacctggtcccggcagctatcaaacccaaaccaatagcccccgccgtgcgcgtagtgttgcctctcaatccattatagcatacaccatgtctttgggcgccgagaatagcgtgggccccggaccaggcaatactagtaatcaggtcgccgtactgtatcaaggtgtgaattgtaccgaggtccccgtcgcaatccacgcagatcaattgactccaacatggcgggttggaccgggacctggaacatctcgctattgggaacctgaattttacgaagcgatgtacacaccccacacagtcttgcaggccgttggagcttgtgttctctgtaattcccaaacaggaccggggcccggtattacaagtgggtggacctttggagctggcgcagcactccagatacctttcgcaatgcaaatggcataccggtttaatggaataggggtaacgcagaacggtccgggaccgggcgcgtatgcgaacagaaatagatttctctatataattaaactaatcttcctgtggctcctctggccagtcaccttggcctgcttcgtgcttgccgccgtcggccctgggcccgggaaattgcttgagcaatggaacctcgttatcgggttcttgttcctgacgtggatttgtctactgcaattcgcttatgctaaccgcaatagattcctttatggccccggacctggcggggtccctgttgtggactcctattattctctcctgatgcccatactcactttgacacgggccctcactgccgaaagtcacgtggacacagatctaacaggccccggccccggttttcgcctgactctcggggtctacgattatctcgtgagcacccaagagtttcgctatatgaactctcaagggctgcttccgcccaagaactcaatcgatggaccaggtccgggcaaatatgagcagtacattaagtggccctggtacatttggttgggatttatcgctggcctcattgcaattgtcatggttactatcatgctgtgttgtatgggcccaggtccaggactcgtcggactcatgtggctgtcctacttcatcgcgtcttttagactctttgcaaggacacgttccatgtggagttttaaccctgaaactaatattctgggtcctggtcccgggatagctatgagcgcctttgccatgatgtttgtcaaacataaacatgcttttctttgcttgttcctcttgcctagtctagcaaccgtggcctacttcaatggcccagggcccggaatccctaatcccttgttgggtcttgattgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 74 - DNA, 5079 bp, Artificial Sequence
  Synthetic polynucleotide
  promoter (137)..(695): CMV promoter
  sig_peptide (724)..(795): lysosomal signal (LAMP1 SP (m))
  misc_feature (796)..(810): linker
  misc_feature (811)..(849): PADRE adjuvant
  misc_feature (850)..(864): linker
  misc_feature (865)..(963): epitope S_339 (SEQ ID NO 45)
  misc_feature (964)..(978): GPGPG linker
  misc_feature (979)..(1077): epitope S_252 (SEQ ID NO 41)
  misc_feature (1078)..(1092): GPGPG linker
  misc_feature (1093)..(1191): epitope S_806 (SEQ ID NO 62)
  misc_feature (1192)..(1206): GPGPG linker
  misc_feature (1207)..(1305): epitope S_462 (SEQ ID NO 64)
  misc_feature (1306)..(1320): GPGPG linker
  misc_feature (1321)..(1419): epitope N_305 (SEQ ID NO 60)
  misc_feature (1420)..(1434): GPGPG linker
  misc_feature (1435)..(1533): epitope S_1081 (SEQ ID NO 59)
  misc_feature (1534)..(1548): GPGPG linker
  misc_feature (1549)..(1647): epitope S_673 (SEQ ID NO 47)
  misc_feature (1648)..(1662): GPGPG linker
  misc_feature (1663)..(1761): epitope S_603 (SEQ ID NO 104)
  misc_feature (1762)..(1776): GPGPG linker
  misc_feature (1777)..(1875): epitope ORF1ab_5304 (SEQ ID NO 33)
  misc_feature (1876)..(1890): GPGPG linker
  misc_feature (1891)..(1989): epitope S_882 (SEQ ID NO 17)
  misc_feature (1990)..(2004): GPGPG linker
  misc_feature (2005)..(2103): epitope M_38 (SEQ ID NO 32)
  misc_feature (2104)..(2118): GPGPG linker
  misc_feature (2119)..(2217): epitope M_15 (SEQ ID NO 31)
  misc_feature (2218)..(2232): GPGPG linker
  misc_feature (2233)..(2331): epitope ORF1ab_4622 (SEQ ID NO 24)
  misc_feature (2332)..(2346): GPGPG linker
  misc_feature (2347)..(2445): epitope ORF1ab_3804 (SEQ ID NO 8)
  misc_feature (2446)..(2460): GPGPG linker
  misc_feature (2461)..(2559): epitope S_1205 (SEQ ID NO 34)
  misc_feature (2560)..(2574): GPGPG linker
  misc_feature (2575)..(2673): epitope M_87 (SEQ ID NO 5)
  misc_feature (2674)..(2688): GPGPG linker
  misc_feature (2689)..(2787): epitope ORF1ab_3619 (SEQ ID NO 1)
  misc_feature (2788)..(2802): GPGPG linker
  misc_feature (2803)..(2829): V5 tag
  polyA_site (2909)..(3133): BGH/pA
  misc_feature (3306)..(4100): Kanamicyn resistance
  misc_feature (4400)..(5073): pUC origin
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggcagccccaggtgcacgacgacccctgctactcctactactcgcaggcctggcacacggtgcaagtgctgaagccgctgctaaggcaaagttcgtcgcagcctggacattgaaagcagcagccgaggcagccgcaaagggcgaggtcttcaacgcaactaggtttgcttcagtctatgcttggaaccgcaagcggatctctaactgcgtggctgactatagtgtcttgtataatagtggaccaggtcccggtggagactcatccagcgggtggaccgctggagcagccgcttattatgtgggttatctccaacctcgcactttcttgctcaagtacaatgaaaacggaacaggacctggacccggtcttcctgacccttctaagccatccaagagatcctttatagaagacttgctgttcaacaaggttactcttgctgatgcagggtttatcaaacaatacggcggacctggcccaggaaagccctttgaaagggatatcagtactgagatttaccaagctgggtctaccccttgtaatggtgttgaagggtttaattgctacttccctctccagagtggtcctgggcctggggcccaattcgctccaagcgccagtgccttcttcggaatgtctaggatcggtatggaagtcactcctagtgggacatggcttacctacactggggccattggcccaggcccaggtatctgccacgatggtaaagcccattttccccgtgaaggcgtctttgtttctaatgggacacattggtttgtgactcaacgtaacttttatgaaccacaaggacctggtcccggcagctatcaaacccaaaccaatagcccccgccgtgcgcgtagtgttgcctctcaatccattatagcatacaccatgtctttgggcgccgagaatagcgtgggccccggaccaggcaatactagtaatcaggtcgccgtactgtatcaaggtgtgaattgtaccgaggtccccgtcgcaatccacgcagatcaattgactccaacatggcgggttggaccgggacctggaacatctcgctattgggaacctgaattttacgaagcgatgtacacaccccacacagtcttgcaggccgttggagcttgtgttctctgtaattcccaaacaggaccggggcccggtattacaagtgggtggacctttggagctggcgcagcactccagatacctttcgcaatgcaaatggcataccggtttaatggaataggggtaacgcagaacggtccgggaccgggcgcgtatgcgaacagaaatagatttctctatataattaaactaatcttcctgtggctcctctggccagtcaccttggcctgcttcgtgcttgccgccgtcggccctgggcccgggaaattgcttgagcaatggaacctcgttatcgggttcttgttcctgacgtggatttgtctactgcaattcgcttatgctaaccgcaatagattcctttatggccccggacctggcggggtccctgttgtggactcctattattctctcctgatgcccatactcactttgacacgggccctcactgccgaaagtcacgtggacacagatctaacaggccccggccccggttttcgcctgactctcggggtctacgattatctcgtgagcacccaagagtttcgctatatgaactctcaagggctgcttccgcccaagaactcaatcgatggaccaggtccgggcaaatatgagcagtacattaagtggccctggtacatttggttgggatttatcgctggcctcattgcaattgtcatggttactatcatgctgtgttgtatgggcccaggtccaggactcgtcggactcatgtggctgtcctacttcatcgcgtcttttagactctttgcaaggacacgttccatgtggagttttaaccctgaaactaatattctgggtcctggtcccgggatagctatgagcgcctttgccatgatgtttgtcaaacataaacatgcttttctttgcttgttcctcttgcctagtctagcaaccgtggcctacttcaatggcccagggcccggaatccctaatcccttgttgggtcttgattgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 75 - protein, 13 aa, Artificial Sequence
  Synthetic polypeptide
  misc_feature (3)..(3): Xaa can be any naturally occurring amino acid
AKXVAAWTLKAAA

SEQ ID NO: 76 - protein, 21 aa, Artificial Sequence
  Synthetic polypeptide
FNNFTVSFWLRVPKVSASHLE

SEQ ID NO: 77 - protein, 9 aa, Artificial Sequence
  Synthetic polypeptide
FVNEFYAYL

SEQ ID NO: 78 - protein, 33 aa, Artificial Sequence
  Synthetic polypeptide
ISNSWLMWLIINLVQMAPISAMVRMYIFFASFY

SEQ ID NO: 79 - protein, 5 aa, Artificial Sequence
  Synthetic polypeptide
GPGPG

SEQ ID NO: 80 - protein, 12 aa, Artificial Sequence
  Synthetic polypeptide
HEYGAEALERAG

SEQ ID NO: 81 - protein, 5 aa, Artificial Sequence
  Synthetic polypeptide
EAAAK

SEQ ID NO: 82 - protein, 10 aa, Artificial Sequence
  Synthetic polypeptide
FVSNGTHWFV

SEQ ID NO: 83 - protein, 9 aa, Artificial Sequence
  Synthetic polypeptide
SHMYCSFYP

SEQ ID NO: 84 - protein, 9 aa, Artificial Sequence
  Synthetic polypeptide
SHFYCDVGP

SEQ ID NO: 85 - protein, 9 aa, Artificial Sequence
  Synthetic polypeptide
YRFPPATTP

SEQ ID NO: 86 - protein, 10 aa, Artificial Sequence
  Synthetic polypeptide
YRFHWIHTPD

SEQ ID NO: 87 - protein, 8 aa, Artificial Sequence
  Synthetic polypeptide
RFWLFFLP

SEQ ID NO: 88 - protein, 9 aa, Artificial Sequence
  Synthetic polypeptide
FHWYKDLLP

SEQ ID NO: 89 - protein, 9 aa, Artificial Sequence
  Synthetic polypeptide
SHPPQIVLP

SEQ ID NO: 90 - protein, 9 aa, Artificial Sequence
  Synthetic polypeptide
DRDPAAFAP

SEQ ID NO: 91 - protein, 9 aa, Artificial Sequence
  Synthetic polypeptide
SHTYGMTIP

SEQ ID NO: 92 - protein, 9 aa, Artificial Sequence
  Synthetic polypeptide
SHSPPLNLP

SEQ ID NO: 93 - protein, 9 aa, Artificial Sequence
  Synthetic polypeptide
SHFYCDVGP

SEQ ID NO: 94 - protein, 9 aa, Artificial Sequence
  Synthetic polypeptide
SHFYCDSLP

SEQ ID NO: 95 - protein, 9 aa, Artificial Sequence
  Synthetic polypeptide
DRFWLFFLP

SEQ ID NO: 96 - protein, 9 aa, Artificial Sequence
  Synthetic polypeptide
SHFSYSLSP

SEQ ID NO: 97 - protein, 9 aa, Artificial Sequence
  Synthetic polypeptide
SHFYCDDVP

SEQ ID NO: 98 - protein, 9 aa, Artificial Sequence
  Synthetic polypeptide
MHIYGSYSP

SEQ ID NO: 99 - protein, 15 aa, Artificial Sequence
  Synthetic polypeptide
SGPGPGGDSSSGWTA

SEQ ID NO: 100 - protein, 15 aa, Artificial Sequence
  Synthetic polypeptide
SGWTAGAAAYYVGYL

SEQ ID NO: 101 - protein, 15 aa, Artificial Sequence
  Synthetic polypeptide
QSAPHGVVFAAYICH

SEQ ID NO: 102 - protein, 15 aa, Artificial Sequence
  Synthetic polypeptide
FPREGVFVSNGTHWF

SEQ ID NO: 103 - protein, 15 aa, Artificial Sequence
  Synthetic polypeptide
VFVSNGTHWFVTQRN

SEQ ID NO: 104 - protein, 33 aa, Artificial Sequence
  Synthetic polypeptide
NTSNQVAVLYQGVNCTEVPVAIHADQLTPTWRV